(19) United States
(12) Patent Application Publication
(10) Pub. No.: US 2010/0190787 A1
(43) Pub. Date: Jul. 29, 2010

(54) Modulators of Mitotic Kinases

(76) Inventors: Srinivas Rao Kasibhatla, San Diego, CA (US); Kevin Hong, San Diego, CA (US); Lin Zhang, San Diego, CA (US); Marcus F. Boehm, San Diego, CA (US); Junhua Fan, San Diego, CA (US); Jean-Yves LeBrazidec, San Diego, CA (US)

Correspondence Address: Jonathan P. O''Brien, Ph.D.;Honigman Miller Schwartz and Cohn, 350 East Michigan Avenue, Suite 300, KALAMAZOO, MI, 49007, US

(21) Appl. No.: 12/525,155

(22) PCT Filed: Jan. 30, 2008
(86) PCT No.: PCT/US08/01195

§ 371 Date: Mar. 22, 2010

Related U.S. Application Data
(60) Provisional application No. 60/898,382, filed on Jan. 30, 2007.
(60) Provisional application No. 60/898,300, filed on Jan. 30, 2007.

(51) Int. Cl. A61K 31/5377; C07D 473/00; A61K 31/52; C07D 413/02

(52) U.S. Cl. 514/234.2; 544/277; 514/263.4; 544/118

(57) ABSTRACT

The invention relates to compounds of Formula (I), a prodrug, a polymorph, a tautomer, an enantiomer, a stereoisomer, a solvate, an N-oxide, or a pharmaceutically acceptable salt thereof: (formula should be inserted here) which have inhibitory effect on one or more protein kinases that are involved in cell mitosis.

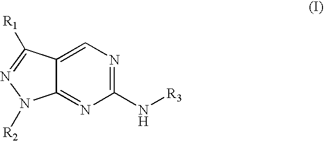

### CROSS-REFERENCE

This application claims priority to U.S. Application No. 60/898,300, filed on Jan. 30, 2007, and U.S. Application No. 60/898,382, also filed on Jan. 30, 2007. Both of these applications are incorporated herein by reference in their entirety.

### BACKGROUND OF THE INVENTION

Mitosis is an extraordinarily complex biological process in which a complete copy of the duplicated genome is precisely segregated by the microtubule spindle apparatus into two daughter cells. Since the survival of a cell depends on the accuracy of mitosis, multiple fidelity monitoring checkpoint systems have evolved to ensure correct temporal and spatial coordination of this process. Errors in these mechanisms can lead to genomic instability, an important aspect of tumorigenesis. As a result, it is not surprising that these regulatory systems are frequently found to be abnormal in tumor cells when compared to normal cells. See, e.g., Keen, N. et al., Nature Rev. Cancer, 4: 927-936 (2004); Lengauer, C. et al., Nature, 396, 643-649 (1998).

The onset and end of mitosis are tightly regulated by the phosphorylation and dephosphorylation of numerous proteins. The mitotic phosphorylations are carried out by multiple mitotic serine/threonine kinases such as Aurora kinases, polo-like kinases, cyclin-dependent kinases, and MIMA. See, e.g., Nigg, E. A., Nature Rev. Mol. Cell. Biol., 2: 21-32 (2001); Toji, S. et al., Genes to Cells, 9: 383-397 (2004).

Eleven forms of cyclin-dependent kinases (CDKs) are known to exist and are named as CDK1 through CDK11. Most of the CDKs were originally discovered as being involved in the regulation of the cell cycle. CDKs are also involved in the regulation of transcription and mRNA processing. CDKs are considered a potential target for anti-cancer medication. Cell death may be caused by interfering with CDK action selectively to interrupt the cell cycle regulation in cancer cells. Currently, some CDK inhibitors such as Seliciclib are undergoing clinical trials. See, e.g., Loyer, P. et al., Cellular Signalling, 17 (9): 1033-51 (2005); Adriano G Rossi, Nature Medicine, 12: 1056-1064 (2006).

Aurora kinases are protein serine/threonine kinases essential to mitotic progression. In mammalian cells, the Aurora kinase family consists of three members, namely, Aurora A, Aurora B, and Aurora C. These kinases share a conserved catalytic domain and participate in regulating mitotic processes although there exist some differences in their subcellular localization and mitotic functions. See, e.g., Brown, J. R. et al., BMC Evol. Biol., 4: 39 (2004). Aurora A is localized to duplicated centrosomes and spindle poles during mitosis. Functional studies show that this protein is required for centrosome maturation, separation and mitotic spindle formation. Suppression of Aurora A expression by RNA interference (RNAi) delays mitotic entry in human cells and over-expression of this kinase compromises spindle-checkpoint function and inhibits cytokinesis. Aurora B is a chromosome passenger protein, which is localized to the centromeric region of chromosomes in early stages of mitosis; it translocates to the spindle equator and the spindle midzone during anaphase A, and to the midbody between anaphase B and cytokinesis. It is believed that this protein is actively involved in regulating chromosome alignment and segregation, spindle-checkpoint function, and cytokinesis. Over-expression of kinase dead Aurora B protein blocks the attachment of chromosomes to mitotic spindles, strongly suggestive of defective kinetochores. In addition, impaired functions of Aurora B as the result of RNAi or antibody injection result in spindle checkpoint failure because the cells are unable to undergo mitotic arrest in response to exposure to nocodazole and paclitaxel. Aurora C is a centrosome-associated kinase that may also play a role in the development and progression of cancer. See Jiang, N. et al., Mini-Reviews in Medicianl Chemistry, 6: 885-895 (2006). Deregulated expression of Aurora kinases is closely associated with tumorigenesis. Many studies show that Aurora A and Aurora B genes are either over-expressed or amplified in a broad range of tumors. See, e.g., Keen, N. et al., supra; Anand, S. et al., Cancer Cell, 3: 51 (2003); Warner, S. L. et al., Mol. Cancer. Ther., 2: 589 (2003). Due to the important role of Aurora kinases in the mitosis as well as tumorigenesis, great efforts have been directed to the development of compounds targeting these molecules.

Polo-like kinases (PLKs) are a family of protein serine/threonine kinases highly conserved among eukaryotes. In mammals, the PLK family consists of four members, namely, PLK1, PLK2, PLK3, and PLK4. In addition to the conserved kinase domain, PLKs share unique motifs termed Polo box domains (PBDs) that are present in the C-terminus of this group of proteins. See, e.g., Xie, S. Q. et al., Oncogene, 24: 277 (2005). Several studies show that PBDs play a critical role in regulation of the subcellular localization probably through interaction with certain phosphorylated proteins critical for cell proliferation. See, e.g., Lowery, D. M. et al., Oncogene, 24: 248 (2005). PLKs have multiple functions in regulating the cell cycle, especially during mitosis. See, Xie, S. Q. et al., supra. PLKs are able to activate the Cdk1/cyclinB complex, which is the key molecule to initiation of mitosis entry. PLK1 also phosphorylates components of the anaphase-promoting complex such as Cdc16 and Cdc27, suggesting that PLK1 is an important regulator of metaphase and anaphase transition. PLKs are also required for completion of mitosis since point mutations or N-terminal truncation cause cytokinesis failure. See, e.g., van Vugt, M. A. et al., Oncogene, 24: 2844 (2005). Over-expression of PLK1 is detected in a majority of human tumor cells and tumor cell lines. See, e.g., Eckerdt, F. et al., Oncogene, 24, 267 (2005); Takai, N. et al., Oncogene, 24: 287 (2005). In addition, elevated PLK1 expression is closely associated with poor prognosis in cancer patients as well as the metastatic potential of certain tumors. Thus, it is believed that PLK1 plays a causative role in oncogenic transformation. See Takai, N. et al., supra.

In view of roles that multiple mitotic kinases play in tumor cell division and proliferation, it is desirable to have compounds that can inhibit one or more of these multiple mitotic kinases for treating cancer.

### SUMMARY OF THE INVENTION

One aspect of this invention relates to compounds of Formula (I), a prodrug, a polymorph, a tautomer, an enantiomer, a stereoisomer, a solvate, an N-oxide, or a pharmaceutically acceptable salt thereof.

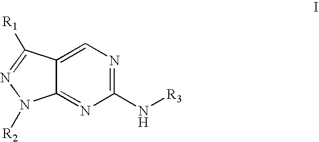

Referring to Formula (I),

R¹ is hydrogen or halo;

R² is -L₁-Rₐ, wherein
L₁ is a bond and Rₐ is alkenyl, alkynyl, cycloalkyl, heterocycloalkyl, aryl, or heteroaryl; in which each of cycloalkyl, heterocycloalkyl, aryl, and heteroaryl is optionally substituted and has 8 to 16 ring atoms; or
L₁ is alkyl and Rₐ is hydrogen, alkenyl, alkynyl, amino, hydroxy, alkoxycarbonyl, alkylcarbonyloxy, cycloalkyl, heterocycloalkyl, aryl, or heteroaryl; in which each of cycloalkyl, heterocycloalkyl, aryl, and heteroaryl is optionally substituted and has 8 to 16 ring atoms;

R³ is -R_{b}-L₂-R_{c}; wherein
R_{b} is aryl, heteroaryl, cycloalkyl, or heterocycloalkyl, and is optionally substituted with 1 to 3 substituents; wherein two of the substituents when adjacent, together with the atom or atoms to which they are attached, can form a 5- to 16-membered ring with 0 to 6 hetero ring atoms,
L₂ is a bond, —(CRₓR_{y})ₙ—, —N═, —O—, —S—, —SO—, —SO₂—, —CO—, —CO—O—, —O—CO—, —NRₓ—, —NRₓ—CO—, —NRₓ—SO₂—, —CO—NRₓ—, —SO₂—NRₓ—, —NRₓ—CO—O—, —NRₓ—SO₂—O—, —NRₓ—CO—NR_{y}—, —NRₓ—SO₂—NR_{y}—, —CO—NRₓ—NR_{y}—, —CO—NRₓ—NR_{y}—, —SO₂—NRₓ—NR_{y}—, —NRₓ—CO—CO—O—, —NRₓ—SO₂—SO₂—O—, —S(O)₂—Nₓ—CO—R_{y}—, —CO—N_{X}—S(O)₂—R_{y}—, or —(NRₓR_{y})C═N—O—;
R_{c} is hydrogen, alkyl, alkenyl, alkynyl, cycloalkyl, heterocycloalkyl, cycloalkenyl, heterocycloalkenyl, aryl, heteroaryl, cycloalkyl-alkyl, heterocycloalkyl-alkyl, cycloalkenyl-alkyl, heterocycloalkenyl-alkyl, aralkyl, or heteroaralkyl; and, except for hydrogen, is optionally substituted with 1 to 3 substituents;
each of Rₓ and R_{y}, independently, is hydrogen, hydroxy, alkyl, alkoxy, amino, —CO-alkyl, —CO-aryl, —SO₂-alkyl, —SO₂-aryl, —SO₂-heteroaryl, or —P(O)(O-alkyl)₂, wherein the alkyl or aryl moiety in Rₓ or R_{y} is optionally substituted with 1 to 3 substituents; and
n is 0, 1, 2, or 3.

Note that the orientation of L₂ is as shown above, i.e., the left bond of each Markush member links to R_{b} and the right bond links to R_{c}. For instance, when L₂ is —CO—O—, R₃ is —R_{b}—CO—O—R_{c}—.

In some embodiments, R_{b} is optionally substituted with 1 to 3 substituents each independently selected from the group consisting of alkyl, alkenyl, alkynyl, cycloalkyl, heteroaryl, aryl, heteroaryl, arylalkyl, heteroarylalkyl, —OR, —SR, —NRR′, oxo, —C(O)—OR, —C(O)—NRR′, halo, —CN, —NO₂, —N₃, —C(O)R, —P(O)(OR)R′, —O—P(O)(OR)R′, —NR—P(O)(OR′)R″, —S(O)₂—(OR), —O—S(O)₂—(OR), —NR—S(O)₂—OR′, —NR—C(O)—OR′, —NR—C(O)—NR′R″, —NR—C(S)—NR′R″, —C(S)—NRR′, and thioalkyl; each of R and R′, independently, is hydrogen, alkyl, cycloalkyl, heterocycloalkyl, aryl, heteroaryl, aralkyl, or heteroaralkyl; and R″ is alkyl, cycloalkyl, heterocycloalkyl, aryl, heteroaryl, aralkyl, or heteroaralkyl.

In some embodiments, R_{c} is, except when being hydrogen, optionally substituted with 1 to 3 substituents each independently selected from the group consisting of alkyl, alkenyl, alkynyl, cycloalkyl, heteroaryl, aryl, heteroaryl, arylalkyl, heteroarylalkyl, —OR, —SR, —NRR′, oxo, —C(O)—OR, —C(O)—NRR′, halo, —CN, —NO₂, —N₃, —C(O)R, —P(O)(OR)R′, —O—P(O)(OR)R′, —NR—P(O)(OR′)R″, —S(O)₂—(OR), —O—S(O)₂—(OR), —NR—S(O)₂—OR′, —NR—C(O)—OR′, —NR—C(O)—NR′R″,

—NR—C(S)—NR′R″, —C(S)—NRR′, and thioalkyl; each of R and R′, independently, is hydrogen, alkyl, cycloalkyl, heterocycloalkyl, aryl, heteroaryl, aralkyl, or heteroaralkyl; and R″ is alkyl, cycloalkyl, heterocycloalkyl, aryl, heteroaryl, aralkyl, or heteroaralkyl.

In some embodiments, the alkyl or aryl moiety in Rₓ or R_{y} is optionally substituted with 1 to 3 substituents each independently selected from the group consisting of alkyl, alkenyl, alkynyl, cycloalkyl, heteroaryl, aryl, heteroaryl, arylalkyl, heteroarylalkyl, —OR, —SR, —NRR′, oxo, —C(O)—OR, —C(O)—NRR′, halo, —CN, —NO₂, —N₃, —C(O)R, —P(O)(OR)R′, —O—P(O)(OR)R′, —NR—P(O)(OR′)R″, —S(O)₂—(OR), —O—S(O)₂—(OR), —NR—S(O)₂—OR′, —NR—C(O)—OR′, —NR—C(O)—NR′R″, —NR—C(S)—NR′R″, —C(S)—NRR′, and thioalkyl; each of R and R′, independently, is hydrogen, alkyl, cycloalkyl, heterocycloalkyl, aryl, heteroaryl, aralkyl, or heteroaralkyl; and R″ is alkyl, cycloalkyl, heterocycloalkyl, aryl, heteroaryl, aralkyl, or heteroaralkyl.

In some embodiments, R₁ is hydrogen.

In some embodiments, L₁ is a bond.

In some embodiments, Rₐ is cycloalkyl, heterocycloalkyl, aryl, or heteroaryl, each having 8 to 12 ring atoms. Unless otherwise specified, these cycloalkyl, heterocyclolakyl, aryl, or heteraryl groups can be connected, at any ring atoms, to the linker (L₁) or the ring nitrogen atom (when L₁ is a bond). Where applicable, the connecting ring atom can have an R, S, or racemic confirgulation.

In some embodiments, Rₐ is dihydroindenyl (also called “indanyl”)

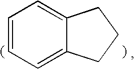

tetrahydronaphthyl

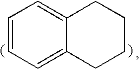

dihydroacenaphthylenyl

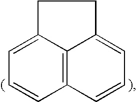

naphthyl

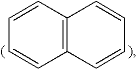

tetrahydro-benzo[7]annulenyl

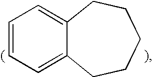

quinolinyl

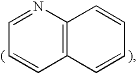

isoquinolinyl

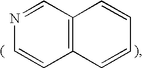

benzimidazolyl

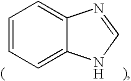

benzothiazolyl

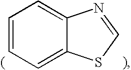

benzoxazolyl

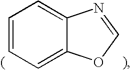

phenanthrenyl

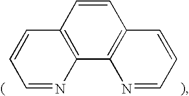

or tetrahydro-dibenzo[7]annulenyl

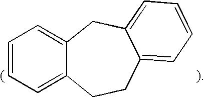

As mentioned above, each of these rings can be connected at any ring atom to the linker (L₁) or, when L₁ is a bond, to the nitrogen atom of the pyrazolyl ring to which R₂ is attached.

In some embodiments, Rₐ is dihydroindenyl, tetrahydronaphthyl, dihydroacenaphthylenyl, or naphthyl.

In some embodiments, Rₐ is naphthyl or dihydroindenyl (e.g., naphth-1-yl, 1,2-dihydroacenaphthylen-1-yl, 2,3-dihydroinden-2-yl, or 2,3-dihydroinden-1-yl).

In some embodiments, L₁ is a bond.

In some embodiments, Rₐ is tetrahydronaphthyl optionally substituted with 1-3 substituents. Each of the 1-3 optional substituents can be independently selected from the group consisting of benzyloxy, hydroxy, and methylsulfonyloxy.

In some further embodiments, Rₐ is 1-benzyloxy-5,6,7,8-tetrahydronaphthalene-4-yl; 1-hydroxy-5,6,7,8-tetrahydronaphthalene-4-yl; or 1-methanesulfonyloxy-5,6,7,8-tetrahydronaphthalene-4-yl.

In some embodiments, Rₐ is dihydroindenyl optionally substituted with 1 to 3 substitutents. Suitable substituents include, but are not limited to, hydroxy, benzoyloxy, dihydrophosphoryloxy, alkylenedioxo, acetamido, di-tert-butylphosphoryloxy, alkoxoalkoxy (e.g., methoxyethoxy), and alkoxy (e.g., methoxy).

In some further embodiments, Rₐ is dihydroinden-1-yl, 4-hydroxydihydroinden-1-yl, 4-benzoyloxydihydroinden-1-yl, 4-dihydrophosphoryloxydihydroinden-1-yl, 5,6-dioxoledihydroinden-1-yl, 5-acetamidodihydroinden-1-yl, 5-benzoyloxydihydroinden-1-yl, 4-benzoyloxydihydroinden-1-yl, 4-(di-tert-butylphosphoryloxy)dihydroinden-1-yl, 5-((2-methoxyethoxy)methyl)dihydroinden-1-yl, or 5-methoxydihydroinden-1-yl.

In some embodiments, Rₐ is indolyl optionally substituted with 1 to 3 substituents. Suitable substituents include, but are not limited to, alkyl (e.g., methyl) and alkylcarboxy (e.g., tert-butylcarboxy).

In some further embodiments, Rₐ is indol-4-yl, indol-6-yl, indol-5-yl, 7-methylindol-5-yl, 1-tert-butylcarboxy-7-methylindol-5-yl, or (1-tert-butylcarboxy)indol-5-yl.

In some embodiments, R₂ is 2,3-dihydro-1H-inden-1-yl, (S)-2,3-dihydro-1H-inden-1-yl, (R)-2,3-dihydro-1H-inden-1-yl, 1,2-dihydroacenaphthylen-1-yl, (R)-1,2-dihydroacenaphthylen-1-yl, (S)-1,2-dihydroacenaphthylen-1-yl, phenanthren-1-yl, or phenanthren-4-yl.

In some embodiments, L₁ is alkyl and Rₐ is hydrogen, alkenyl, alkynyl, amino, hydroxy, alkoxycarbonyl, alkylcarbonyloxy, cycloalkyl, heterocycloalkyl, aryl, or heteroaryl; in which each of cycloalkyl, heterocycloalkyl, aryl, and heteroaryl is optionally substituted with 1-3 substituents and has 8 to 16 ring atoms. For instance, L₁ can be methyl, ethyl, propyl, isopropyl, or butyl; and Rₐ can be hydrogen, amino, hydroxy, alkoxycarbonyl, alkylcarbonyloxy, cycloalkyl, heterocycloalkyl, aryl, or heteroaryl; in which each of cycloalkyl, heterocycloalkyl, aryl, and heteroaryl is optionally substituted with 1-3 substituents.

In some further embodiments, R₂ is methyl, ethyl, propyl, isopropyl, butyl, 3-aminopropyl, hydroxybutyl, ethoxycarbonylmethyl, methylcarbonyloxybutyl, or naphthalen-1-ylmethyl.

In still some further embodiments, R₃ is phenyl optionally substituted with 1-3 substitutents. For instance, phenyl can be substituted at the para-position. Examples of such phenyl include 4-(piperidin-1-yl)phenyl and 4-(bis(methylsulfonyl)amino)phenyl.

In some embodiments, R_{b} is phenyl and is optionally substituted with 1 to 3 substituents.

In some embodiments, L₂ is —O—, —S—, —SO₂—, —CO—, —CO—, —CO—O—, —NRₓ—, —NRₓ—CO—, —NRₓ—SO₂—, —NRₓ—CO—O—, —NRₓ—CO—NR_{y}—, or —NRₓ—CO—CO—O—.

In some embodiments, L₂ is —CO—O—, —NRₓ—, —NRₓ—SO₂—, —NRₓ—CO—O—, or —NRₓ—CO—NR_{y}— with Rₓ being hydrogen, alkyl, —CO-alkyl, —SO₂-alkyl, —SO₂-aryl, or —SO₂-heteroaryl.

In some embodiments, R_{c} is hydrogen, alkyl, aryl, or heteroaryl.

In some embodiments, Rₓ is hydrogen, alkyl, —CO-alkyl, or —SO₂-alkyl; and R_{c} is hydrogen, alkyl, or aryl.

In some embodiments, L₂ is a bond; and R_{c} is cycloalkyl, heterocycloalkyl, aryl, heteroaryl, (cycloalkyl)alkyl, (heterocycloalkyl)alkyl, aralkyl, or heteroaralkyl.

In some embodiments, L₂ is a bond; and R_{c} is heterocycloalkyl, heteroaryl, heterocycloalkyl-alkyl, or heteroaralkyl.

In some embodiments, L₂ is a bond and R_{c} is tetrazolyl, morpholino, or piperazinyl.

In some embodiments, R_{c} is cycloalkyl, heterocycloalkyl, aryl, heteroaryl, cycloalkyl-alkyl, heterocycloalkyl-alkyl, aralkyl, or heteroaralkyl.

In some embodiments, L₁ is a bond; Rₐ is cycloalkyl, heterocycloalkyl, aryl, and heteroaryl; R_{b} is phenyl; L₂ is —O—, —S—, —SO₂—, —CO—, —CO—O—, —NRₓ—, —NRₓ—CO—, —NRₓ—SO₂—, —NRₓ—CO—O—, —NRₓ—CO—NR_{y}—, or —NRₓ—CO—CO—O— with Rₓ being hydrogen, alkyl, —CO-alkyl, —SO₂-alkyl, —SO₂-aryl, or —SO₂-heteroaryl; and R_{c} is hydrogen, alkyl, aryl, or heteroaryl.

In some embodiments, L₁ is a bond; Rₐ is dihydroindenyl, tetrahydronaphthyl, dihydroacenaphthylenyl, or naphthyl; R_{b} is phenyl; L₂ is —CO—O—, —NRₓ—, —NRₓ—SO₂—, —NRₓ—CO—O—, or —NRₓ—CO—NR_{y}—, with Rₓ being hydrogen, alkyl, —CO-alkyl, —SO₂-alkyl, or —SO₂-aryl; and R_{c} is hydrogen, alkyl, or aryl.

In some embodiments, L₁ is a bond; Rₐ is cycloalkyl, heterocycloalkyl, aryl, and heteroaryl having 8 to 12 ring atoms; R_{b} is phenyl; L₂ is a bond; and R_{c} is cycloalkyl, heterocycloalkyl, aryl, heteroaryl, (cycloalkyl)alkyl, (heterocycloalkyl)alkyl, aralkyl, or heteroaralkyl.

In some embodiments, L₁ is a bond; Rₐ is dihydroindenyl, tetrahydronaphthyl, dihydroacenaphthylenyl, or naphthyl; R_{b} is phenyl; L₂ is a bond; and R_{c} is tetrazolyl, morpholino, or piperazinyl.

In some embodiments, L₂ is a bond or —NRₓ—SO₂—; Rₓ is hydrogen or —SO₂-alkyl; and R_{c} is hydrogen, alkyl, heterocycloalkyl, heteroaryl, heterocycloalkyl-alkyl, or heteroaralkyl.

In some embodiments, Rₓ is —SO₂-alkyl; and R_{c} is alkyl.

In some embodiments, R_{c} is tetrazolyl, morpholino, or piperazinyl.

In some embodiments, R_{b} is phenyl substituted with 1 to 3 substituents each independently being —NRR′ or —C(O)OR; L₂ is a bond; and R_{c} is hydrogen.

In some embodiments, L₁ is a bond and Rₐ is dihydroindenyl, tetrahydronaphthyl, dihydroacenaphthylenyl, naphthyl, tetrahydro-benzo[7]annulenyl, quinolinyl, isoquinolinyl, benzimidazolyl, benzothiazolyl, benzoxazolyl, phenanthrenyl, or tetrahydro-dibenzo[7]annulenyl.

In some embodiments, R_{b} is phenyl.

In some embodiments, L₂ is a bond or —NRₓ—SO₂— with Rₓ being hydrogen or —SO₂-alkyl; and R_{c} is hydrogen, alkyl, heterocycloalkyl, heteroaryl, heterocycloalkyl-alkyl, or heteroaralkyl.

In some embodiments, R_{c} is hydrogen, alkyl, heterocycloalkyl, heteroaryl, heterocycloalkyl-alkyl, or heteroaralkyl.

In some embodiments, R_{b} is phenyl substituted with 1 to 3 substitutents each independently being —NRR′ or —C(O)OR; L₂ is a bond; and R_{c} is hydrogen.

In some embodiments, R₁ is hydrogen.

In some embodiments, R_{b} is phenyl optionally substituted with —NRR′ or C(O)OR; L₂ is a bond or —NRₓ—SO₂— with Rₓ being hydrogen or —SO₂-alkyl; and R_{c} is tetrazolyl, morpholino, or piperazinyl.

In some embodiments, R₃ is (4-(1H-tetrazol-5-yl)phenyl), 3-hydroxy-4-methoxyphenyl, 4-(4-methylpiperazin-1-yl)phenyl), 4-(piperazin-1-yl)phenyl, 4-aminophenyl, 4-benzoic acid, 4-morpholinophenyl, 4-(N,N-dimethylsulfonamide)phenyl, or 4-(methanesulfonamide)phenyl.

In some other embodiments, R₃ is 4-bis(methanesulfonyl)aminophenyl, 4-bis(ethanesulfonyl)aminophenyl, 4-(4-methylpiperizin-1-yl)phenyl, 4-bis(methanesulfonyl)aminophenyl, 4-bis((chloromethane)sulfonyl)aminophenyl, 4-(methanesulfonyl)aminophenyl, 4-(N′-hydroxycarbamimidoyl)phenyl, 4-(4-methylpiperizin-1-yl)phenyl, 4-aminophenyl, 4-(methanesulfonylcarbamoyl)phenyl, 4-cyanophenyl, 4-(tetrazol-5-yl)aminophenyl, 2-(4-nitrophenyl)ethyl, 2-(4-aminophenyl)ethyl, or 2-(4-bis(methanesulfonyl)aminophenyl)ethyl.

In still some other embodiments, R₃ is 4-bis(methanesulfonyl)aminophenyl.

In some embodiments, the compound is:
N-(methylsulfonyl)-N-(4-(1-(naphthalen-1-yl)-1H-pyrazolo[3,4-d]pyrimidin-6-ylamino)phenyl)methanesulfonamide,
N-methyl-N-(4-(1-(naphthalen-1-yl)-1H-pyrazolo[3,4-d]pyrimidin-6-ylamino)phenyl)methanesulfonamide,
N-methyl-N-(4-(1-(naphthalen-1-yl)-1H-pyrazolo[3,4-d]pyrimidin-6-ylamino)phenyl)methanesulfonamide,
1-(methylsulfonyl)-N-(4-(1-(naphthalen-1-yl)-1H-pyrazolo[3,4-d]pyrimidin-6-ylamino)phenyl)methanesulfonamide,
N¹-(1-(naphthalen-2-yl)-1H-pyrazolo[3,4-d]pyrimidin-6-yl)benzene-1,4-diamine,
ethyl-2-(4-(1-(naphthalen-1-yl)-1H-pyrazolo[3,4-d]pyrimidin-6-ylamino)phenylamino)-2-oxoacetate,
N-(4-(1-(naphthalen-2-yl)-1H-pyrazolo[3,4-d]pyrimidin-6-ylamino)phenyl)methanesulfonamide,
3,3,3-trifluoro-N-(4-(1-(naphthalen-1-yl)-1H-pyrazolo[3,4-d]pyrimidin-6-ylamino)phenyl)propane-1-sulfonamide,
methyl-4-(1-(naphthalen-1-yl)-1H-pyrazolo[3,4-d]pyrimidin-6-ylamino)phenylcarbamate,
N-(3,4-dimethoxyphenyl)-1-(naphthalen-1-yl)-1H-pyrazolo[3,4-d]pyrimidin-6-amine,
N-(4-(4-methylpiperazin-1-yl)phenyl)-1-(naphthalen-2-yl)-1H-pyrazolo[3,4-d]pyrimidin-6-amine,
ethyl-2-hydroxy-4-(1-(naphthalen-1-yl)-1H-pyrazolo[3,4-d]pyrimidin-6-ylamino)phenylcarbamate,
N-(4-(1H-1,2,4-triazol-1-yl)phenyl)-1-(naphthalen-1-yl)-1H-pyrazolo[3,4-d]pyrimidin-6-amine,
methyl3-(4-(1-(naphthalen-1-yl)-1H-pyrazolo[3,4-d]pyrimidin-6-ylamino)phenylamino)-3-oxopropanoate,
N¹-(1-(naphthalen-1-yl)-1H-pyrazolo[3,4-d]pyrimidin-6-yl)benzene-1,4-diamine,
N-(4-(4-methylpiperazin-1-yl)phenyl)-1-(naphthalen-1-yl)-1H-pyrazolo[3,4-d]pyrimidin-6-amine,
N-(4-fluoro-3-methoxyphenyl)-1-(naphthalen-1-yl)-1H-pyrazolo[3,4-d]pyrimidin-6-amine,
N-(4-ethoxyphenyl)-1-(naphthalen-1-yl)-1H-pyrazolo[3,4-d]pyrimidin-6-amine,
(S)-1-(2,3-dihydro-1H-inden-1-yl)-N-(4-(4-methylpiperazin-1-yl)phenyl)-1H-pyrazolo[3,4-d]pyrimidin-6-amine,
(S)-4-(1-(2,3-dihydro-1H-inden-1-yl)-1H-pyrazolo[3,4-d]pyrimidin-6-ylamino)benzoic acid,
(S)—N-(4-(1-(2,3-dihydro-1H-inden-1-yl)-1H-pyrazolo[3,4-d]pyrimidin-6-ylamino)phenyl)-N-(methylsulfonyl)methanesulfonamide,
(S)—N¹-(1-(2,3-dihydro-1H-inden-1-yl)-1H-pyrazolo[3,4-d]pyrimidin-6-yl)benzene-1,4-diamine,
(S)-1-(2,3-dihydro-1H-inden-1-yl)-N-(4-morpholinophenyl)-1H-pyrazolo[3,4-d]pyrimidin-6-amine,
(S)-1-(2,3-dihydro-1H-inden-1-yl)-N-(4-(4-(propylsulfonyl)piperazin-1-yl)phenyl)-1H-pyrazolo[3,4-d]pyrimidin-6-amine,
4-(1-(2,3-dihydro-1H-inden-1-yl)-1H-pyrazolo[3,4-d]pyrimidin-6-ylamino)benzoic acid,
1-(2,3-dihydro-1H-inden-1-yl)-N-(3-(piperazin-1-yl)phenyl)-1H-pyrazolo[3,4-d]pyrimidin-6-amine,

N¹-(1-(2,3-dihydro-1H-inden-1-yl)-1H-pyrazolo[3,4-d]pyrimidin-6-yl)benzene-1,4-diamine,
N-(4-(1-(2,3-dihydro-1H-inden-1-yl)-1H-pyrazolo[3,4-d]pyrimidin-6-ylamino)phenyl)methanesulfonamide,
1-(2,3-dihydro-1H-inden-1-yl)-N-(4-(4-(propylsulfonyl)piperazin-1-yl)phenyl)-1H-pyrazolo[3,4-d]pyrimidin-6-amine,
(S)-1-(2,3-dihydro-1H-inden-1-yl)-N-(4-(4-(methylsulfonyl)piperazin-1-yl)phenyl)-1H-pyrazolo[3,4-d]pyrimidin-6-amine,
(S)-1-(2,3-dihydro-1H-inden-1-yl)-N-(4-(piperazin-1-yl)phenyl)-1H-pyrazolo[3,4-d]pyrimidin-6-amine,
N-(4-(1-(2,3-dihydro-1H-inden-1-yl)-1H-pyrazolo[3,4-d]pyrimidin-6-ylamino)phenyl)benzamide,
(S)-5-(1-(2,3-dihydro-1H-inden-1-yl)-1H-pyrazolo[3,4-d]pyrimidin-6-ylamino)-2-methoxyphenol,
1-(2,3-dihydro-1H-inden-1-yl)-N-(4-(piperazin-1-yl)phenyl)-1H-pyrazolo[3,4-d]pyrimidin-6-amine,
1-(2,3-dihydro-1H-inden-1-yl)-N-(4-morpholinophenyl)-1H-pyrazolo[3,4-d]pyrimidin-6-amine,
3-(4-(4-(1-(2,3-dihydro-1H-inden-1-yl)-1H-pyrazolo[3,4-d]pyrimidin-6-ylamino)phenyl)piperazin-1-yl)propan-1-ol,
4-(1-(2,3-dihydro-1H-inden-1-yl)-1H-pyrazolo[3,4-d]pyrimidin-6-ylamino)benzohydrazide,
2-(4-(4-(1-(2,3-dihydro-1H-inden-1-yl)-1H-pyrazolo[3,4-d]pyrimidin-6-ylamino)phenyl)piperazin-1-yl)ethylacetate,
1-(2,3-dihydro-1H-inden-1-yl)-N-(4-(4-((4-methoxy-3,5-dimethylpyridin-2-yl)methyl)piperazin-1-yl)phenyl)-1H-pyrazolo[3,4-d]pyrimidin-6-amine,
3-(4-(4-(1-(2,3-dihydro-1H-inden-1-yl)-1H-pyrazolo[3,4-d]pyrimidin-6-ylamino)phenyl)piperazin-1-yl)propylacetate,
2-(4-(4-(1-(2,3-dihydro-1H-inden-1-yl)-1H-pyrazolo[3,4-d]pyrimidin-6-ylamino)phenyl)piperazin-1-yl)ethanol,
1-((S)-2,3-dihydro-1H-inden-1-yl)-N-(3-(1-((R)-2,3-dihydro-1H-inden-1-yl)-1H-pyrazolo[3,4-d]pyrimidin-6-yloxy)-4-methoxyphenyl)-1H-pyrazolo[3,4-d]pyrimidin-6-amine,
(S)-ethyl4-(1-(2,3-dihydro-1H-inden-1-yl)-1H-pyrazolo[3,4-d]pyrimidin-6-ylamino)benzoate,
(S)-1-(2,3-dihydro-1H-inden-1-yl)-N-(4-morpholinophenyl)-1H-pyrazolo[3,4-d]pyrimidin-6-amine,
N-(4-(1-(4-hydroxy-2,3-dihydro-1H-inden-1-yl)-1H-pyrazolo[3,4-d]pyrimidin-6-ylamino)phenyl)-N-(methylsulfonyl)methanesulfonamide,
(S)—N-(4-(1-(2,3-dihydro-1H-inden-1-yl)-1H-pyrazolo[3,4-d]pyrimidin-6-ylamino)phenyl)-N-(ethylsulfonyl)ethanesulfonamide,
1-(6-(4-(4-methylpiperazin-1-yl)phenylamino)-1H-pyrazolo[3,4-d]pyrimidin-1-yl)-2,3-dihydro-1H-inden-4-ol,
1-(6-(4-(N-(methylsulfonyl)methylsulfonamido)phenylamino)-1H-pyrazolo[3,4-d]pyrimidin-1-yl)-2,3-dihydro-1H-inden-4-yl benzoate,
1-(6-(4-(N-(methylsulfonyl)methylsulfonamido)phenylamino)-1H-pyrazolo[3,4-d]pyrimidin-1-yl)-2,3-dihydro-1H-inden-4-yl dihydrogen phosphate,
(S)-1-chloro-N-(chloromethylsulfonyl)-N-(4-(1-(2,3-dihydro-1H-inden-1-yl)-1H-pyrazolo[3,4-d]pyrimidin-6-ylamino)phenyl)methanesulfonamide,
N-(4-(1-(6,7-dihydro-5H-indeno[5,6-d][1,3]dioxol-5-yl)-1H-pyrazolo[3,4-d]pyrimidin-6-ylamino)phenyl)-N-(methylsulfonyl)methanesulfonamide,
N-(1-(6-(4-(N-(methylsulfonyl)methylsulfonamido)phenylamino)-1H-pyrazolo[3,4-d]pyrimidin-1-yl)-2,3-dihydro-1H-inden-5-yl)acetamide,
N-(4-(1-(4-hydroxy-2,3-dihydro-1H-inden-1-yl)-1H-pyrazolo[3,4-d]pyrimidin-6-ylamino)phenyl)methanesulfonamide,
1-(6-(4-(N-(methylsulfonyl)methylsulfonamido)phenylamino)-1H-pyrazolo[3,4-d]pyrimidin-1-yl)-2,3-dihydro-1H-inden-5-yl benzoate,
(S,Z)-4-(1-(2,3-dihydro-1H-inden-1-yl)-1H-pyrazolo[3,4-d]pyrimidin-6-ylamino)-N′-hydroxybenzimidamide,
1-(6-(4-(4-methylpiperazin-1-yl)phenylamino)-1H-pyrazolo[3,4-d]pyrimidin-1-yl)-2,3-dihydro-1H-inden-4-yl benzoate,
1-(6-(4-aminophenylamino)-1H-pyrazolo[3,4-d]pyrimidin-1-yl)-2,3-dihydro-1H-inden-4-ol,
1-(6-(4-aminophenylamino)-1H-pyrazolo[3,4-d]pyrimidin-1-yl)-2,3-dihydro-1H-inden-4-yl benzoate,
di-tert-butyl 1-(6-(4-(N-(methylsulfonyl)methylsulfonamido)phenylamino)-1H-pyrazolo[3,4-d]pyrimidin-1-yl)-2,3-dihydro-1H-inden-4-yl phosphate,
(S)-4-(1-(2,3-dihydro-1H-inden-1-yl)-1H-pyrazolo[3,4-d]pyrimidin-6-ylamino)-N-(methylsulfonyl)benzamide,
N-(4-(1-(5-((2-methoxyethoxy)methoxy)-2,3-dihydro-1H-inden-1-yl)-1H-pyrazolo[3,4-d]pyrimidin-6-ylamino)phenyl)-N-(methylsulfonyl)methanesulfonamide,
N-(1-(6-(4-aminophenylamino)-1H-pyrazolo[3,4-d]pyrimidin-1-yl)-2,3-dihydro-1H-inden-5-yl)acetamide,
(S)-4-(1-(2,3-dihydro-1H-inden-1-yl)-1H-pyrazolo[3,4-d]pyrimidin-6-ylamino)benzonitrile,
(S)—N-(4-(1H-tetrazol-5-yl)phenyl)-1-(2,3-dihydro-1H-inden-1-yl)-1H-pyrazolo[3,4-d]pyrimidin-6-amine,
N1-(1-(5-methoxy-2,3-dihydro-1H-inden-1-yl)-1H-pyrazolo[3,4-d]pyrimidin-6-yl)benzene-1,4-diamine,
N-(4-(1-(5-methoxy-2,3-dihydro-1H-inden-1-yl)-1H-pyrazolo[3,4-d]pyrimidin-6-ylamino)phenyl)-N-(methylsulfonyl)methanesulfonamide,
1-(2,3-dihydro-1H-inden-2-yl)-N-(4-morpholinophenyl)-1H-pyrazolo[3,4-d]pyrimidin-6-amine,
N-(4-(1-(1H-indol-4-yl)-1H-pyrazolo[3,4-d]pyrimidin-6-ylamino)phenyl)-N-(methylsulfonyl)methanesulfonamide,
N-(4-(1-(1H-indol-6-yl)-1H-pyrazolo[3,4-d]pyrimidin-6-ylamino)phenyl)-N-(methylsulfonyl)methanesulfonamide,
N-(4-(1-(1H-indol-5-yl)-1H-pyrazolo[3,4-d]pyrimidin-6-ylamino)phenyl)-N-(methylsulfonyl)methane sulfonamide,
1-(1H-indol-4-yl)-N-(4-(4-methylpiperazin-1-yl)phenyl)-1H-pyrazolo[3,4-d]pyrimidin-6-amine,
N-(4-(1-(1-methyl-1H-indol-5-yl)-1H-pyrazolo[3,4-d]pyrimidin-6-ylamino)phenyl)-N-(methylsulfonyl)methanesulfonamide,
1-(7-methyl-1H-indol-4-yl)-N-(4-(4-methylpiperazin-1-yl)phenyl)-1H-pyrazolo[3,4-d]pyrimidin-6-amine,
tert-butyl 7-methyl-4-(6-(4-(4-methylpiperazin-1-yl)phenylamino)-1H-pyrazolo[3,4-d]pyrimidin-1-yl)-1H-indole-1-carboxylate,
tert-butyl 4-(6-(4-(4-methylpiperazin-1-yl)phenylamino)-1H-pyrazolo[3,4-d]pyrimidin-1-yl)-1H-indole-1-carboxylate,
1-(1,2-dihydroacenaphthylen-1-yl)-N-(4-(4-methylpiperazin-1-yl)phenyl)-1H-pyrazolo[3,4-d]pyrimidin-6-amine,
N-(4-(1-(1,2-dihydroacenaphthylen-1-yl)-1H-pyrazolo[3,4-d]pyrimidin-6-ylamino)phenyl)-N-(methylsulfonyl)methanesulfonamide,
N¹-(1-(1,2-dihydroacenaphthylen-1-yl)-1H-pyrazolo[3,4-d]pyrimidin-6-yl)benzene-1,4-diamine,
1-(1,2-dihydroacenaphthylen-1-yl)-N-(4-morpholinophenyl)-1H-pyrazolo[3,4-d]pyrimidin-6-amine,
4-(1-(1,2-dihydroacenaphthylen-1-yl)-1H-pyrazolo[3,4-d]pyrimidin-6-ylamino)benzonitrile,
1-(1,2-dihydroacenaphthylen-1-yl)-N-(4-(2-(trimethylstannyl)-2H-tetrazol-5-yl)phenyl)-1H-pyrazolo[3,4-d]pyrimidin-6-amine,
1-(1,2-dihydroacenaphthylen-1-yl)-N-(4-(piperazin-1-yl)phenyl)-1H-pyrazolo[3,4-d]pyrimidin-6-amine,
N-(4-morpholinophenyl)-1-(naphthalen-1-ylmethyl)-1H-pyrazolo[3,4-d]pyrimidin-6-amine,
N-(4-(1-(4-(benzyloxy)-5,6,7,8-tetrahydronaphthalen-1-yl)-1H-pyrazolo[3,4-d]pyrimidin-6-ylamino)phenyl)-N-(methylsulfonyl)methanesulfonamide,
1-(4-(benzyloxy)-5,6,7,8-tetrahydronaphthalen-1-yl)-N-(4-(4-methylpiperazin-1-yl)phenyl)-1H-pyrazolo[3,4-d]pyrimidin-6-amine,
N1-(1-(4-(benzyloxy)-5,6,7,8-tetrahydronaphthalen-1-yl)-1H-pyrazolo[3,4-d]pyrimidin-6-yl)benzene-1,4-diamine,
4-(6-(4-aminophenylamino)-1H-pyrazolo[3,4-d]pyrimidin-1-yl)-5,6,7,8-tetrahydronaphthalen-1-ol,
tert-butyl 4-(1-(4-(benzyloxy)-5,6,7,8-tetrahydronaphthalen-1-yl)-1H-pyrazolo[3,4-d]pyrimidin-6-ylamino)piperidine-1-carboxylate,
4-(6-(4-(N-(methylsulfonyl)methylsulfonamido)phenylamino)-1H-pyrazolo[3,4-d]pyrimidin-1-yl)-5,6,7,8-tetrahydronaphthalen-1-yl methanesulfonate,
4-(6-(4-(methylsulfonamido)phenylamino)-1H-pyrazolo[3,4-d]pyrimidin-1-yl)-5,6,7,8-tetrahydronaphthalen-1-yl methanesulfonate,
N-(4-(1-(4-(benzyloxy)-5,6,7,8-tetrahydronaphthalen-1-yl)-1H-pyrazolo[3,4-d]pyrimidin-6-ylamino)phenyl)methanesulfonamide,
N-(4-(1-(4-hydroxy-5,6,7,8-tetrahydronaphthalen-1-yl)-1H-pyrazolo[3,4-d]pyrimidin-6-ylamino)phenyl)-N-(methylsulfonyl)methanesulfonamide,
N-(4-(1-(4-hydroxy-5,6,7,8-tetrahydronaphthalen-1-yl)-1H-pyrazolo[3,4-d]pyrimidin-6-ylamino)phenyl)methanesulfonamide,
N-(4-morpholinophenyl)-1-(1,2,3,4-tetrahydronaphthalen-1-yl)-1H-pyrazolo[3,4-d]pyrimidin-6-amine,
4-(6-(4-(4-methylpiperazin-1-yl)phenylamino)-1H-pyrazolo[3,4-d]pyrimidin-1-yl)-5,6,7,8-tetrahydronaphthalen-1-ol,
1-(4-(benzyloxy)-5,6,7,8-tetrahydronaphthalen-1-yl)-N-(4-morpholinophenyl)-1H-pyrazolo[3,4-d]pyrimidin-6-amine, or
4-(6-(4-morpholinophenylamino)-1H-pyrazolo[3,4-d]pyrimidin-1-yl)-5,6,7,8-tetrahydronaphthalen-1-ol.

In still some embodiments, the compound is
N-(4-(1H-tetrazol-5-yl)phenyl)-1-((S)-(2,3-dihydro-1H-inden-1-yl))-1H-pyrazolo[3,4-d]pyrimidin-6-amine,
N-(4-(1H-tetrazol-5-yl)phenyl)-1-((R)-(2,3-dihydro-1H-inden-1-yl))-1H-pyrazolo[3,4-d]pyrimidin-6-amine,
N-(4-(1H-tetrazol-5-yl)phenyl)-1-(2,3-dihydro-1H-inden-1-yl)-1H-pyrazolo[3,4-d]pyrimidin-6-amine,
N-(4-(1H-tetrazol-5-yl)phenyl)-1-((S)-(1,2-dihydro acenaphthyl en-1-yl))-1H-pyrazolo[3,4-d]pyrimidin-6-amine,
N-(4-(1H-tetrazol-5-yl)phenyl)-1-((R)-(1,2-dihydro acenaphthyl en-1-yl))-1H-pyrazolo[3,4-d]pyrimidin-6-amine,
N-(4-(1H-tetrazol-5-yl)phenyl)-1-(1,2-dihydroacenaphthylen-1-yl)-1H-pyrazolo[3,4-d]pyrimidin-6-amine,
N-(4-(1H-tetrazol-5-yl)phenyl)-1-(1-naphthyl)-1H-pyrazolo[3,4-d]pyrimidin-6-amine,
N-(4-(1H-tetrazol-5-yl)phenyl)-1-(phenanthren-4-yl)-1H-pyrazolo[3,4-d]pyrimidin-6-amine,
N-(4-morpholinophenyl)-1-((S)-(2,3-dihydro-1H-inden-1-yl))-1H-pyrazolo[3,4-d]pyrimidin-6-amine,
N-(4-morpholinophenyl)-1-((R)-(2,3-dihydro-1H-inden-1-yl))-1H-pyrazolo[3,4-d]pyrimidin-6-amine,
N-(4-morpholinophenyl)-1-(2,3-dihydro-1H-inden-1-yl)-1H-pyrazolo[3,4-d]pyrimidin-6-amine,
N-(4-morpholinophenyl)-1-((S)-(1,2-dihydroacenaphthylen-1-yl))-1H-pyrazolo[3,4-d]pyrimidin-6-amine,
N-(4-morpholinophenyl)-1-((R)-(1,2-dihydroacenaphthylen-1-yl))-1H-pyrazolo[3,4-d]pyrimidin-6-amine,
N-(4-morpholinophenyl)-1-(1,2-dihydroacenaphthylen-1-yl)-1H-pyrazolo[3,4-d]pyrimidin-6-amine,
N-(4-morpholinophenyl)-1-(1-naphthyl)-1H-pyrazolo[3,4-d]pyrimidin-6-amine,
N-(4-morpholinophenyl)-1-(phenanthren-4-yl)-1H-pyrazolo[3,4-d]pyrimidin-6-amine,
N-(4-(piperazin-1-yl)phenyl)-1-((S)-(2,3-dihydro-1H-inden-1-yl))-1H-pyrazolo[3,4-d]pyrimidin-6-amine,
N-(4-(piperazin-1-yl)phenyl)-1-((R)-(2,3-dihydro-1H-inden-1-yl))-1H-pyrazolo[3,4-d]pyrimidin-6-amine,
N-(4-(piperazin-1-yl)phenyl)-1-(2,3-dihydro-1H-inden-1-yl)-1H-pyrazolo[3,4-d]pyrimidin-6-amine,
N-(4-(piperazin-1-yl)phenyl)-1-((S)-(1,2-dihydroacenaphthylen-1-yl))-1H-pyrazolo[3,4-d]pyrimidin-6-amine,
N-(4-(piperazin-1-yl)phenyl)-1-((R)-(1,2-dihydroacenaphthylen-1-yl))-1H-pyrazolo[3,4-d]pyrimidin-6-amine,
N-(4-(piperazin-1-yl)phenyl)-1-(1,2-dihydroacenaphthylen-1-yl)-1H-pyrazolo[3,4-d]pyrimidin-6-amine,
N-(4-(piperazin-1-yl)phenyl)-1-(1-naphthyl)-1H-pyrazolo[3,4-d]pyrimidin-6-amine,
N-(4-(piperazin-1-yl)phenyl)-1-(phenanthren-4-yl)-1H-pyrazolo[3,4-d]pyrimidin-6-amine,
(S)-4-(1-(2,3-dihydro-1H-inden-1-yl)-1H-pyrazolo[3,4-d]pyrimidin-6-ylamino)benzoic acid,
(R)-4-(1-(2,3-dihydro-1H-inden-1-yl)-1H-pyrazolo[3,4-d]pyrimidin-6-ylamino)benzoic acid,
4-(1-(2,3-dihydro-1H-inden-1-yl)-1H-pyrazolo[3,4-d]pyrimidin-6-ylamino)benzoic acid,
(S)-4-(1-(1,2-dihydroacenaphthylen-1-yl)-1H-pyrazolo[3,4-d]pyrimidin-6-ylamino)benzoic acid,
(R)-4-(1-(1,2-dihydroacenaphthylen-1-yl)-1H-pyrazolo[3,4-d]pyrimidin-6-ylamino)benzoic acid,
4-(1-(1,2-dihydroacenaphthylen-1-yl)-1H-pyrazolo[3,4-d]pyrimidin-6-ylamino)benzoic acid,
4-(1-(1-napthyl)-1H-pyrazolo[3,4-d]pyrimidin-6-ylamino)benzoic acid,
4-(1-(phenanthren-4-yl)-1H-pyrazolo[3,4-d]pyrimidin-6-ylamino)benzoic acid,
N-(4-aminophenyl)-1-((S)-(2,3-dihydro-1H-inden-1-yl))-1H-pyrazolo[3,4-d]pyrimidin-6-amine,
N-(4-aminophenyl)-1-((R)-(2,3-dihydro-1H-inden-1-yl))-1H-pyrazolo[3,4-d]pyrimidin-6-amine,
N-(4-aminophenyl)-1-(2,3-dihydro-1H-inden-1-yl)-1H-pyrazolo[3,4-d]pyrimidin-6-amine,
N-(4-aminophenyl)-1-((S)-(1,2-dihydroacenaphthylen-1-yl))-1H-pyrazolo[3,4-d]pyrimidin-6-amine,
N-(4-aminophenyl)-1-((R)-(1,2-dihydroacenaphthylen-1-yl))-1H-pyrazolo[3,4-d]pyrimidin-6-amine,
N-(4-aminophenyl)-1-(1,2-dihydroacenaphthylen-1-yl)-1H-pyrazolo[3,4-d]pyrimidin-6-amine,
N-(4-aminophenyl)-1-(1-naphthyl)-1H-pyrazolo[3,4-d]pyrimidin-6-amine,
N-(4-aminophenyl)-1-(phenanthren-4-yl)-1H-pyrazolo[3,4-d]pyrimidin-6-amine,
(S)—N-(4-(1-(2,3-dihydro-1H-inden-1-yl)-/H-pyrazolo[3,4-d]pyrimidin-6-ylamino) phenyl)-N-(methylsulfonyl)methanesulfonamide,
(R)—N-(4-(1-(2,3-dihydro-1H-inden-1-yl)-/H-pyrazolo[3,4-d]pyrimidin-6-ylamino) phenyl)-N-(methylsulfonyl)methanesulfonamide,
N-(Methylsulfonyl)-N-(4-(1-(2,3-dihydro-1H-inden-1-yl)-1H-pyrazolo[3,4-d]pyrimidin-6-ylamino)phenyl)methanesulfonamide,
(S)—N-(4-(1-(1,2-dihydroacenaphthylen-1-yl)-/H-pyrazolo[3,4-d]pyrimidin-6-ylamino) phenyl)-N-(methylsulfonyl)methanesulfonamide,
(R)—N-(4-(1-(1,2-dihydro acenaphthyl en-1-yl)-/H-pyrazolo[3, 4-d]pyrimidin-6-ylamino) phenyl)-N-(methylsulfonyl)methanesulfonamide,
N-(4-(1-(1,2-dihydroacenaphthylen-1-yl)-/H-pyrazolo[3,4-d]pyrimidin-6-ylamino) phenyl)-N-(methylsulfonyl)methanesulfonamide,
N-(4-(1-(1-naphthyl)-/H-pyrazolo[3,4-d]pyrimidin-6-ylamino)phenyl)-N-(methylsulfonyl)methanesulfonamide,
N-(4-(1-(phenanthren-4-yl)-/H-pyrazolo[3,4-d]pyrimidin-6-ylamino)phenyl)-N-(methylsulfonyl)methanesulfonamide,
(S)—N-(4-(1-(2,3-dihydro-1H-inden-1-yl)-1H-pyrazolo[3,4-d]pyrimidin-6-ylamino)phenyl)methanesulfonamide,
(R)—N-(4-(1-(2,3-dihydro-1H-inden-1-yl)-1H-pyrazolo[3,4-d]pyrimidin-6-ylamino)phenyl)methanesulfonamide,
N-(4-(1-(2,3-dihydro-1H-inden-1-yl)-1H-pyrazolo[3,4-d]pyrimidin-6-ylamino)phenyl)methanesulfonamide,
(S)—N-(4-(1-(1,2-dihydroacenaphthylen-1-yl)-/H-pyrazolo[3,4-d]pyrimidin-6-ylamino) phenyl)methanesulfonamide,
(R)—N-(4-(1-(1,2-dihydroacenaphthylen-1-yl)-/H-pyrazolo[3,4-d]pyrimidin-6-ylamino) phenyl)methanesulfonamide,
N-(4-(1-(1,2-dihydroacenaphthylen-1-yl)-/H-pyrazolo[3,4-d]pyrimidin-6-ylamino) phenyl)methanesulfonamide,
N-(4-(1-(1-naphthyl)-/H-pyrazolo[3,4-d]pyrimidin-6-ylamino) phenyl)methanesulfonamide,
N-(4-(1-(phenanthren-4-yl)-/H-pyrazolo[3,4-d]pyrimidin-6-ylamino) phenyl)methanesulfonamide,
N-4-(4-methylpiperazin-1-yl)phenyl-1-(S)-(2,3-dihydro-1H-inden-1-yl)-1H-pyrazolo[3,4-d]pyrimidin-6-amine,
N-4-(4-methylpiperazin-1-yl)phenyl-1-(R)-(2,3-dihydro-1H-inden-1-yl)-1H-pyrazolo[3,4-d]pyrimidin-6-amine,
N-4-(4-methylpiperazin-1-yl)phenyl-1-(2,3-dihydro-1H-inden-1-yl)-1H-pyrazolo[3,4-d]pyrimidin-6-amine,
N-4-(4-methylpiperazin-1-yl)phenyl-1-(S)-(1,2-dihydro acenaphthyl en-1-yl)-1H-pyrazolo[3,4-d]pyrimidin-6-amine,
N-4-(4-methylpiperazin-1-yl)phenyl-1-(R)-(1,2-dihydroacenaphthylen-1-yl)-1H-pyrazolo[3,4-d]pyrimidin-6-amine,
N-4-(4-methylpiperazin-1-yl)phenyl-1-(1,2-dihydroacenaphthylen-1-yl)-1H-pyrazolo[3,4-d]pyrimidin-6-amine,
N-4-(4-methylpiperazin-1-yl)phenyl-1-(1-naphthyl)-1H-pyrazolo[3,4-d]pyrimidin-6-amine,
N-(4-(1-(phenanthren-4-yl)-/H-pyrazolo[3,4-d]pyrimidin-6-ylamino) phenyl)methanesulfonamide,
N-(3-hydroxy-4-methoxyphenyl)-1-((S)-(2,3-dihydro-1H-inden-1-yl))-1H-pyrazolo[3,4-d]pyrimidin-6-amine,
N-(3-hydroxy-4-methoxyphenyl)-1-((R)-(2,3-dihydro-1H-inden-1-yl))-1H-pyrazolo[3,4-d]pyrimidin-6-amine,
N-(3-hydroxy-4-methoxyphenyl)-1-(2,3-dihydro-1H-inden-1-yl)-1H-pyrazolo[3,4-d]pyrimidin-6-amine,
N-(3-hydroxy-4-methoxyphenyl)-1-((S)-(1,2-dihydroacenaphthylen-1-yl))-1H-pyrazolo[3,4-d]pyrimidin-6-amine,
N-(3-hydroxy-4-methoxyphenyl)-1-((R)-(1,2-dihydroacenaphthylen-1-yl))-1H-pyrazolo[3,4-d]pyrimidin-6-amine,
N-(3-hydroxy-4-methoxyphenyl)-1-(1,2-dihydroacenaphthylen-1-yl)-1H-pyrazolo[3,4-d]pyrimidin-6-amine,
N-(3-hydroxy-4-methoxyphenyl)-1-(1-naphthyl)-1H-pyrazolo[3,4-d]pyrimidin-6-amine, or
N-(3-hydroxy-4-methoxyphenyl)-1-(phenanthren-4-yl)-1H-pyrazolo[3,4-d]pyrimidin-6-amine.

In some further embodiments, the compound is
1-((6-fluoro-4H-benzo[d][1,3]dioxin-8-yl)methyl)-N-(4-morpholinophenyl)-1H-pyrazolo[3,4-d]pyrimidin-6-amine,
1-butyl-N-(4-(piperidin-1-yl)phenyl)-1H-pyrazolo[3,4-d]pyrimidin-6-amine,
ethyl 2-(6-(4-(piperidin-1-yl)phenylamino)-1H-pyrazolo[3,4-d]pyrimidin-1-yl)acetate,
1-methyl-N-(4-(piperidin-1-yl)phenyl)-1H-pyrazolo[3,4-d]pyrimidin-6-amine,
1-isopropyl-N-(4-(piperidin-1-yl)phenyl)-1H-pyrazolo[3,4-d]pyrimidin-6-amine,
N-(4-(1-(3-aminopropyl)-1H-pyrazolo[3,4-d]pyrimidin-6-ylamino)phenyl)-N-(methylsulfonyl)methanesulfonamide,
N-(methylsulfonyl)-N-(4-(1-propyl-1H-pyrazolo[3,4-d]pyrimidin-6-ylamino)phenyl)methanesulfonamide,
N-(4-(1-butyl-1H-pyrazolo[3,4-d]pyrimidin-6-ylamino)phenyl)-N-(methylsulfonyl)methanesulfonamide,
N-(4-(1-(4-hydroxybutyl)-1H-pyrazolo[3,4-d]pyrimidin-6-ylamino)phenyl)-N-(methylsulfonyl)methanesulfonamide,
4-(6-(4-(N-(methylsulfonyl)methylsulfonamido)phenylamino)-1H-pyrazolo[3,4-d]pyrimidin-1-yl)butyl acetate,
N-(4-(1-ethyl-1H-pyrazolo[3,4-d]pyrimidin-6-ylamino)phenyl)-N-(methylsulfonyl)methanesulfonamide,
N-(4-(1-isopropyl-1H-pyrazolo[3,4-d]pyrimidin-6-ylamino)phenyl)-N-(methylsulfonyl)methanesulfonamide,
N-(4-(1-methyl-1H-pyrazolo[3,4-d]pyrimidin-6-ylamino)phenyl)-N-(methylsulfonyl)methanesulfonamide,
N-(5-(1-(4-hydroxy-2,3-dihydro-1H-inden-1-yl)-1H-pyrazolo[3,4-d]pyrimidin-6-ylamino)pyridin-2-yl)methanesulfonamide,
N-(2,3-dihydroxypropyl)-N-(5-(1-(4-hydroxy-2,3-dihydro-1H-inden-1-yl)-1H-pyrazolo[3,4-d]pyrimidin-6-ylamino)pyridin-2-yl)methanesulfonamide,
N-(2,3-dihydroxypropyl)-N-(4-(1-(4-hydroxy-2,3-dihydro-1H-inden-1-yl)-1H-pyrazolo[3,4-d]pyrimidin-6-ylamino)phenyl)methanesulfonamide,
N-(5-(1-(4-hydroxy-2,3-dihydro-1H-inden-1-yl)-1H-pyrazolo[3,4-d]pyrimidin-6-ylamino)pyridin-2-yl)-N-(2-morpholinoethyl)methanesulfonamide,
N-(4-(1-(4-hydroxy-2,3-dihydro-1H-inden-1-yl)-1H-pyrazolo[3,4-d]pyrimidin-6-ylamino)phenyl)-N-(2-morpholinoethyl)methanesulfonamide,
N-(4-(1-(4-hydroxy-2,3-dihydro-1H-inden-1-yl)-1H-pyrazolo[3,4-d]pyrimidin-6-ylamino)phenyl)-N-(3-hydroxypropyl)methanesulfonamide,
N-(4-(1-(4-hydroxy-2,3-dihydro-1H-inden-1-yl)-1H-pyrazolo[3,4-d]pyrimidin-6-ylamino)phenyl)-N-(2-hydroxyethyl)methanesulfonamide,
N-(4-(1-(1-hydroxy-6,7-dihydro-5H-cyclopenta[c]pyridin-5-yl)-1H-pyrazolo[3,4-d]pyrimidin-6-ylamino)phenyl)methanesulfonamide,
N-(4-(1-(1-hydroxy-6,7-dihydro-5H-cyclopenta[c]pyridin-5-yl)-1H-pyrazolo[3,4-d]pyrimidin-6-ylamino)phenyl)-N-(2-hydroxyethyl)methanesulfonamide,
N-(4-(1-(1-hydroxy-6,7-dihydro-5H-cyclopenta[c]pyridin-5-yl)-1H-pyrazolo[3,4-d]pyrimidin-6-ylamino)phenyl)-N-(2-morpholino ethyl)methanesulfonamide,
N-(4-(1-(4-hydroxy-6,7-dihydro-5H-cyclopenta[c]pyridin-7-yl)-1H-pyrazolo[3,4-d]pyrimidin-6-ylamino)phenyl)methanesulfonamide,
N-(4-(1-(4-hydroxy-6,7-dihydro-5H-cyclopenta[c]pyridin-7-yl)-1H-pyrazolo[3,4-d]pyrimidin-6-ylamino)phenyl)-N-(2-hydroxyethyl)methanesulfonamide,
N-(4-(1-(4-hydroxy-6,7-dihydro-5H-cyclopenta[c]pyridin-7-yl)-1H-pyrazolo[3,4-d]pyrimidin-6-ylamino)phenyl)-N-(2-morpholinoethyl)methanesulfonamide,
N-(2,3-dihydroxypropyl)-N-(4-(1-(1-hydroxy-6,7-dihydro-5H-cyclopenta[c]pyridin-5-yl)-1H-pyrazolo[3,4-d]pyrimidin-6-ylamino)phenyl)methanesulfonamide,
N-(2,3-dihydroxypropyl)-N-(4-(1-(4-hydroxy-6,7-dihydro-5H-cyclopenta[c]pyridin-7-yl)-1H-pyrazolo[3,4-d]pyrimidin-6-ylamino)phenyl)methanesulfonamide,
N-(4-(1-(4-hydroxy-6,7-dihydro-5H-cyclopenta[b]pyridin-7-yl)-1H-pyrazolo[3,4-d]pyrimidin-6-ylamino)phenyl)methanesulfonamide,
N-(4-(1-(4-hydroxy-6,7-dihydro-5H-cyclopenta[b]pyridin-7-yl)-1H-pyrazolo[3,4-d]pyrimidin-6-ylamino)phenyl)-N-(2-morpholinoethyl)methanesulfonamide,
N-(4-(1-(4-hydroxy-6,7-dihydro-5H-cyclopenta[b]pyridin-7-yl)-1H-pyrazolo[3,4-d]pyrimidin-6-ylamino)phenyl)-N-(2-hydroxyethyl)methanesulfonamide, or
N-(2,3-dihydroxypropyl)-N-(4-(1-(4-hydroxy-6,7-dihydro-5H-cyclopenta[b]pyridin-7-yl)-1H-pyrazolo[3,4-d]pyrimidin-6-ylamino)phenyl)methanesulfonamide.

The present invention includes within its scope prodrugs. In general, such prodrugs will be functional derivatives of the compounds of Formula (I) which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

Also within the scope of this invention are N-oxide derivatives or pharmaceutically acceptable salts of the compounds of Formula (I). For example, a nitrogen ring atom of the imidazole core ring or a nitrogen-containing heterocyclyl substituent can form an oxide in the presence of a suitable oxidizing agent such as m-chloroperbenzoic acid or H₂O₂.

A compound of Formula (I) that is acidic in nature (e.g., having a carboxyl or phenolic hydroxyl group) can form a pharmaceutically acceptable salt such as a sodium, potassium, calcium, or gold salt. Also within the scope of the invention are salts formed with pharmaceutically acceptable amines such as ammonia, alkyl amines, hydroxyalkylamines, and N-methylglycamine. A compound of Formula (I) can be treated with an acid to form acid addition salts. Examples of such acids include hydrochloric acid, hydrobromic acid, hydroiodic acid, sulfuric acid, methanesulfonic acid, phosphoric acid, p-bromophenyl-sulfonic acid, carbonic acid, succinic acid, citric acid, benzoic acid, oxalic acid, malonic acid, salicylic acid, malic acid, fumaric acid, ascorbic acid, maleic acid, acetic acid, and other mineral and organic acids well known to those skilled in the art. The acid addition salts can be prepared by treating a compound of Formula (I) in its free base form with a sufficient amount of an acid (e.g., hydrochloric acid) to produce an acid addition salt (e.g., a hydrochloride salt). The acid addition salt can be converted back to its free base form by treating the salt with a suitable dilute aqueous basic solution (e.g., sodium hydroxide, sodium bicarbonate, potassium carbonate, or ammonia). Compounds of Formula (I) can also be, e.g., in a form of achiral compounds, racemic mixtures, optically active compounds, pure diastereomers, or a mixture of diastereomers.

The compounds described above exhibit inhibitory on one or more protease kinases that are involved in the motitic cycle of a cell, e.g., a tumor cell. Examples of the protease kinases include, among others, all existing forms of Aurora kinase, cyclin-dependent kinase, or polo-like kinase.

As such, also within the scope of this invention is a pharmaceutical composition which contains one of the compounds described above, and a carrier. These pharmaceutical compositions can be used to treat diseases or conditions mediated by a protease kinase that is involved in the cell mitosis.

Another aspect of this invention relates to a method of treating a subject with a protein kinase-mediated disease, wherein the method includes administering to said subject a pharmaceutically effective amount of one of the compounds described above.

The compounds of this invention also exhibit inhibitory effect on one or more kinases involved in the phosphorylation process in the cells. Thus, the invention is further directed to a method for decreasing the phosphorylation of a protease kinase in a cell, which includes contacting the cell with one of the compounds of this invention.

Another aspect of this invention further relates to a method of inhibiting a protease kinase in a cell, which includes contacting the cell with one of the compounds described above. In some embodiments of this method, the protease kinase is one or more protease kinases involved in cell mitosis. In some other embodiments, the protease kinase is an Aurora kinase, a cyclin-dependant kinase, or a polo-like kinase.

This invention further provides a method for inhibiting the abnormal growth of cells, including transformed cells, by administering an effective amount of a compound of the invention. Abnormal growth of cells refers to cell growth independent of normal regulatory mechanisms (e.g. loss of contact inhibition).

This invention may also provide a method for inhibiting proliferative diseases (i.e., diseases worsened due to the reproduction of cells), both benign and malignant, with said inhibition being accomplished by the administration of an effective amount of the compounds described herein, to a subject in need of such a treatment.

The invention further provides pharmaceutical compositions each containing a compound of this invention as described above and methods of using a compound of Formula (I) for modulating the function of a protease kinase which is involved in cell motosis. Examples of such protease kinases include Aurora kinases (e.g., A, B, and C), cyclin-dependent kinase (any of its 11 forms), and polo-like kinase (any of its existing forms).

The invention still further provides a method of treating or preventing tumor or cancer with a compound of Formula (I) to a subject, e.g. a mammal (and more particularly a human) in need of such treatment. The tumor or cancer can be, e.g., bone cancer (e.g., Ewing's sarcoma, osteosarcoma, chondrosarcoma, or orthopaedics links), brain and CNS tumor (e.g., acoustic neuroma, spinal cord tumor, brain tumor ring of hope), breast cancer, breast cancer, colorectal cancer (e.g., anal cancer), endocrine cancer (e.g., adrenocortical carcinoma, pancreatic cancer (e.g. pancreatic carcinoma such as, for example exocrine pancreatic carcinoma), pituitary cancer, thyroid cancer, parathyroid cancer, thymus cancer, multiple endocrine neoplasia, or other endocrine cancer), gastrointestinal cancer (e.g., stomach cancer, esophageal cancer, small intestine cancer, gall bladder cancer, liver cancer, extra-hepatic bile duct cancer, or gastrointestinal carcinoid tumor), genitourinary cancer (testicular cancer, penile cancer, or prostate cancer), gynaecological cancer (e.g., cervical cancer, ovarian cancer, vaginal cancer, uterus/endometrium cancer, vulva cancer, gestational trophoblastic cancer, fallopian tube cancer, or uterine sarcoma), head and neck cancer (e.g., oral cavity, lip, salivary gland cancer, larynx, hypopharynx, oropharynx cancer, nasal, paranasal, or nasopharynx cancer), leukaemia (e.g., acute lymphocytic leukaemia, acute myeloid leukaemia, chronic lymphocytic leukaemia, chronic myeloid leukaemia, hairy cell leukaemia, acute promyelocytic leukemia, plasma cell leukaemia), lung cancer (e.g., adenocarcinoma, small cell lung cancer, or non-small cell lung cancer), lymphoma (e.g., Hodgkin's Disease, Non-Hodgkin's Lymphoma, AIDS-related Lymphoma), eye cancer (e.g., retinoblastoma or intraocular melanoma), skin cancer (e.g., melanoma, non-melanoma skin cancer or Merkel cell cancer), soft tissue sarcoma (e.g., Kaposi's Sarcoma), urinary system cancer (e.g., kidney cancer, Wilm's tumor, bladder cancer, urethral cancer, or transitional cell cancer), and other types or related disorders (e.g., histiocytosis, mesothelioma, metastatic cancer, carcinoid tumors, neurofibromatosis, germ cell tumors, desmoplasic small round cell tumor, malignant rhabdoid tumor, desmoid tumor, ataxia-telangiectasia, Nijmegen breakage syndrome, Rothmund-Thomson syndrome, Li-Fraumeni Syndrome, von Hipple-Lindau Disease, Beckwith-Wiedemann syndrome, Down's syndrome, Denys-Drash syndrome, WAGR syndrome, or CIN cervical intraepithelial neoplas). The compound can be administered in a suitable manner, e.g., intravenously, subcutenously, orally, parenterally, or topically.

In some other embodiments, the compound is administered in combination with a second therapeutic agent. Examples of such a second therapeutic agent include alkylating agents (e.g., Asaley, AZQ, BCNU, Busulfan, carboxyphthalatoplatinum, CBDCA, CCNU, CHIP, chlorambucil, chlorozotocin, cis-platinum, clomesone, cyanomorpholinodoxorubicin, cyclodisone, dianhydrogalactitol, fluorodopan, hepsulfam, hycanthone, melphalan, methyl CCNU, mitomycin C, mitozolamide, nitrogen mustard, PCNU, piperazine, piperazinedione, pipobroman, porfiromycin, spirohydantoin mustard, teroxirone, tetraplatin, thio-tepa, triethylenemelamine, uracil nitrogen mustard, or Yoshi-864), anitmitotic agents (e.g., allocolchicine, Halichondrin B, colchicines, colchicine derivative, dolastatin 10, maytansine, rhizoxin, taxol, taxol derivative, thiocolchicine, trityl cysteine, vinblastine sulfate, or vincristine sulfate), Topoisomerase I inhibitors (e.g., camptothecin, camptothecin sodium, aminocamptothecin, or camptothecin derivatives), Topoisomerase II inhibitors (e.g., doxorubicin, amonafide, m-AMSA, anthrapyrazole derivative, pyrazoloacridine, bisantrene HCl, daunorubicin, deoxydoxorubicin, mitoxantrone, menogaril, N,N-dibenzyl daunomycin, oxanthrazole, rubidazone, VM-26, VP-16), RNA/DNA antimetabolite (e.g., L-alanosine, 5-azacytidine, 5-fluorouracil, acivicin, aminopterin derivative, aminopterin derivative, aminopterin derivative, an antifol, Baker's soluble antifol, dichlorallyl lawsone, brequinar, florafur (pro-drug), 5,6-dihydro-5-azacytidine, methotrexate, methotrexate derivative, N-(phosphonoacetyl)-L-aspartate (PALA), pyrazofurin, or trimetrexate), DNA antimetabolites (e.g., 3-HP, 2′-deoxy-5-fluorouridine, 5-HP, alpha-TGDR, aphidicolin glycinate, ara-C, 5-aza-2′-deoxycytidine, beta-TGDR, cyclocytidine, guanazole, hydroxyurea, inosine glycodialdehyde, macbecin II, pyrazoloimidazole, thioguanine, or thiopurine).

For purposes of this invention, the chemical elements are identified in accordance with the Periodic Table of the Elements, CAS version, Handbook of Chemistry and Physics, 75th Ed. Additionally, general principles of organic chemistry are described by Thomas Sorrell in Organic Chemistry, University Science Books, Sausalito (1999); and by M. B. Smith and J. March in Advanced Organic Chemistry, 5th Ed., John Wiley & Sons, New York (2001), the entire contents of which are hereby incorporated by reference.

The term “modulating” as used herein means increasing or decreasing, e.g. activity, by a measurable amount. Compounds that modulate the function of protease kinases by increasing their activity or their roles in protein phosphorylation are called agonists. Compounds that modulate the function of protease kinases by decreasing their activity or their roles in protein phosphorylation are called antagonists or inhibitors.

As described herein, compounds of the invention may optionally be substituted with one or more substituents, such as those as generally illustrated above, or as specifically exemplified by particular classes, subclasses, and species of the invention.

As used herein, the term “aliphatic” encompasses alkyl, alkenyl, and alkynyl, each of which is optionally substituted as set forth below. Unless otherwise specified, it encompasses both a branched group (e.g., tert-alkyl such as tert-butyl) or a straight aliphatic chain (e.g., n-alkyl groups, alkenyl groups, or alkynyl groups). A straight aliphatic chain has the basic structure of —(CH₂)v—, wherein v can be any integer, e.g., from 1 to 12 (such as 1 to 6). A branched aliphatic chain is a straight aliphatic chain that is substituted with one or more aliphatic groups. A branched aliphatic chain has the structure -[CQQ′]v- wherein at least one of Q and Q′ is an aliphatic group.

As used herein, an “alkyl” group refers to a saturated aliphatic hydrocarbon group containing 1-8 (e.g., 1-6 or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, and 2-ethylhexyl. An alkyl group can be substituted (i.e., optionally substituted) with one or more substituents. Examples of the substituents include, but are not limited to, halo (to give haloalkyl such as trifluoromethy); cycloaliphatic (e.g., cycloalkyl or cycloalkenyl); heterocycloaliphatic (e.g., heterocycloalkyl or heterocycloalkenyl); aryl; heteroaryl; alkoxy; alkoxycarbonyl; alkylcarboxy; aroyl; heteroaroyl; acyl (e.g., (aliphatic)carbonyl, (cycloaliphatic)carbonyl, or (heterocycloaliphatic)carbonyl); nitro; cyano; amido (e.g., (cycloalkylalkyl)amido, arylamidoo, aralkylamido, (heterocycloalkyl)amido, (heterocycloalkylalkyl)amido, heteroarylamido, heteroaralkylamido alkylamido, cycloalkylamido, heterocycloalkylamido, arylamido, or heteroarylamido); amino (e.g., aliphaticamino, cycloaliphaticamino, or heterocycloaliphaticamino); oxime; sulfonyl (e.g., aliphatic-S(O)₂—); sulfinyl; sulfanyl; sulfoxy; urea; thiourea; sulfonamide; sulfamide; oxo (thus forming a carbonyl group, i.e., —CO—); carboxy; carbamoyl; cycloaliphaticoxy; heterocycloaliphaticoxy; aryloxy; heteroaryloxy; aralkyloxy; heteroarylalkoxy; alkoxycarbonyl; alkylcarbonyloxy; hydroxyl; or cycloaliphatic (alkoxy)phosphoryl. Without limitation, examples of substituted alkyls include carboxyalkyl (such as HOOC-alkyl, alkoxycarbonylalkyl, and alkylcarbonyloxyalkyl); cyanoalkyl; hydroxyalkyl; alkoxyalkyl; acylalkyl; aralkyl; (alkoxyaryl)alkyl; (sulfonylamino)alkyl (e.g., alkyl-S(O)₂-aminoalkyl); aminoalkyl; amidoalkyl; (cycloaliphatic)alkyl; silyl (e.g. trialkylsilyl); and haloalkyl.

As used herein, an “alkenyl” group refers to an aliphatic carbon group that contains 2 to 8 (e.g., 2 to 6 or 2 to 4) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents, such as halo; cycloaliphatic (e.g., cycloalkyl or cycloalkenyl); heterocycloaliphatic (e.g., heterocycloalkyl or heterocycloalkenyl); aryl; heteroaryl; alkoxy; aroyl; heteroaroyl; acyl (e.g., (aliphatic)carbonyl, (cycloaliphatic)carbonyl, or (heterocycloaliphatic)carbonyl); nitro; cyano; amido (e.g., (cycloalkylalkyl)amido, arylamido, aralkylamido, (heterocycloalkyl)amido, (heterocycloalkylalkyl)amido, heteroarylamido, heteroaralkylamido alkylaminocarbonyl, cycloalkylaminocarbonyl, heterocycloalkylaminocarbonyl, arylaminocarbonyl, or heteroarylaminocarbonyl); amino (e.g., aliphaticamino, cycloaliphaticamino, heterocycloaliphaticamino, or aliphaticsulfonylamino); oxime; sulfonyl (e.g., alkyl-S(O)₂—, cycloaliphatic-S(O)₂—, or aryl-S(O)₂—); sulfinyl; sulfanyl; sulfoxy; urea; thiourea; sulfonamide; sulfamide; oxo; carboxy; carbamoyl; cycloaliphaticoxy; heterocycloaliphaticoxy; aryloxy; heteroaryloxy; aralkyloxy; heteroaralkoxy; alkoxycarbonyl; alkylcarbonyloxy; or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-S(O)₂-aminoalkenyl), aminoalkenyl, amidoalkenyl, (cycloaliphatic)alkenyl, and haloalkenyl.

As used herein, an “alkynyl” group refers to an aliphatic carbon group that contains 2 to 8 (e.g., 2 to 6 or 2 to 4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl; heteroaroyl; alkoxy; cycloalkyloxy; heterocycloalkyloxy; aryloxy; heteroaryloxy; aralkyloxy; nitro; carboxy; cyano; halo; hydroxy; sulfo; mercapto; sulfanyl (e.g., aliphatic-S— or cycloaliphatic-S—); sulfinyl (e.g., aliphatic-S(O)— or cycloaliphatic-S(O)—); sulfonyl (e.g., aliphatic-S(O)₂—, aliphaticamino-S(O)₂—, or cycloaliphatic-S(O)₂—); amido (e.g., alkylamido, alkylamido, cycloalkylamido, heterocycloalkylamido, cycloalkylamido, arylamido, arylamido, aralkylamido, (heterocycloalkyl)amido, (cycloalkylalkyl)amido, heteroaralkylamido, heteroarylamido or heteroarylamido); urea; thiourea; sulfonamide; sulfamide; alkoxycarbonyl; alkylcarbonyloxy; cycloaliphatic; heterocycloaliphatic; aryl; heteroaryl; acyl (e.g., (cycloaliphatic)carbonyl or (heterocycloaliphatic)carbonyl); amino (e.g., aliphaticamino); sulfoxy; oxo; carbamoyl; (cycloaliphatic)oxy; (heterocycloaliphatic)oxy; or (heteroaryl)alkoxy.

As used herein, an “amido” encompasses both “aminocarbonyl” and “carbonylamino.” Each of these terms, when used alone or in connection with another group, refers to an amido group such as —N(Rₓ)—C(O)—R_{y} or —C(O)—N(Rₓ)₂, when used terminally; or —C(O)—N(Rₓ)— or —N(Rₓ)—C(O)— when used internally, wherein Rₓ and R_{y} are defined below. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, and cycloalkylamido.

As used herein, an “amino” group refers to —NR_{X}R_{Y} wherein each of R_{X} and R_{Y} is independently hydrogen (or sometimes “H” hereinafter), alkyl, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, arylamino, and diarylamino. When the term “amino” is not the terminal group (e.g., alkylcarbonylamino), it is represented by —NR_{X}— in which R_{X} has the same meaning as defined above.

As used herein, an “aryl” group, used alone or as part of a larger moiety such as in “aralkyl”, “aralkoxy,” or “aryloxyalkyl,” refers to monocyclic (e.g., phenyl); bicyclic (e.g., indenyl, naphthalenyl, tetrahydronaphthyl, benzimidazole, benzothiazole, or tetrahydroindenyl); and tricyclic (e.g., fluorenyl tetrahydrofluorenyl, tetrahydroanthracenyl, or anthracenyl) ring systems in which the monocyclic ring system is aromatic or at least one of the rings in a bicyclic or tricyclic ring system is aromatic. The bicyclic and tricyclic groups include benzofused 2- or 3-membered carbocyclic rings. For instance, a benzofused group includes phenyl fused with two or more C₄₋₈ carbocyclic moieties. An aryl is optionally substituted with one or more substituents. Examples of such substitutents include, but are not limited to, aliphatic (e.g., alkyl, alkenyl, or alkynyl); arylaliphatic (e.g., arylalkyl), cycloaliphatic; (cycloaliphatic)aliphatic; heterocycloaliphatic; (heterocycloaliphatic)aliphatic; aryl; heteroaryl; heteroarylaliphatic (e.g., heteroarylalkyl); alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); azide (i.e., —N₃), nitro; carboxy (e.g., alkoxy-C(O)—); amido; amidoamino (e.g., —NR—C(O)—NRR′); thioamido (e.g., —C(S)—NRR′); thioamidoamino (e.g., —NR—C(S)—NRR′); alkoxyamido (e.g., —NR—C(O)-alkoxy or —C(O)—NR-alkoxy); acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl); sulfonyl (e.g., aliphatic-S(O)₂—, (aliphatic-O)—S(O)₂—O—, or amino-S(O)₂—); sulfonylamino (e.g., —NR—S(O)₂—OR′); sulfinyl (e.g., aliphatic-S(O)— or cycloaliphatic-S(O)—); sulfinylamino; sulfanyl (e.g., aliphatic-S—); cyano; halo; hydroxy; mercapto; sulfoxy; urea; thiourea; sulfonamide; sulfamide; carbamoyl; phosphinio (e.g., —P(O)(OR)R′); phosphonio (e.g., —O—P(O)(OR)R′); phosphinioamino (e.g., —NR—P(O)(OR′)R″); or phosphonioamino (e.g., —NR—P(O)(OR)(OR′)). Each of R, R′ and R″ in the just-mentioned examples can be independently an aliphatic. Alternatively, an aryl can be unsubstituted.

Non-limiting examples of substituted aryls include haloaryl (e.g., mono-, di- (e.g., p,m-dihaloaryl), and (trihalo)aryl); (carboxy)aryl (e.g., (alkoxycarbonyl)aryl, ((aralkyl)carbonyloxy)aryl, and (alkoxycarbonyl)aryl); (amido)aryl (e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl); aminoaryl (e.g., ((alkylsulfonyl)amino)aryl or ((dialkyl)amino)aryl); (cyanoalkyl)aryl; (alkoxy)aryl; (sulfonamide)aryl (e.g., (aminosulfonyl)aryl); (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; and (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are as defined herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents.

Each of the one or more substituents independent can be, e.g., aliphatic (e.g., alkyl, alkenyl, or alkynyl, including carboxyalkyl, hydroxyalkyl, or haloalkyl such as trifluoromethyl); cycloaliphatic (e.g., cycloalkyl or cycloalkenyl); (cycloalkyl)alkyl; heterocycloalkyl; (heterocycloalkyl)alkyl; aryl; heteroaryl; alkoxy; cycloalkyloxy; heterocycloalkyloxy; aryloxy; heteroaryloxy; aralkyloxy; heteroaralkyloxy; aroyl; heteroaroyl; nitro; carboxy; alkoxycarbonyl; alkylcarbonyloxy; amido (e.g., alkylamido, cycloalkylamido, (cycloalkylalkyl)amido, arylamido, aralkylamido, (heterocycloalkyl)amido, (heterocycloalkylalkyl)amido, heteroarylamido, or heteroaralkylamido); cyano; halo; hydroxy; acyl; mercapto; alkylsulfanyl; sulfoxy; urea; thiourea; sulfonamide; sulfamide; oxo; or carbamoyl.

As used herein, a “bicyclic ring system” includes 8- to 12- (e.g., 9-, 10-, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bi-cyclic (fused or bridged) ring of 3 to 10 (e.g., 5 to 10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, azacycloalkyl, or ((aminocarbonyl)cycloalkyl)cycloalkyl.

A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, or bicyclo[3.3.1]nonenyl.

A cycloalkyl or cycloalkenyl group can be optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic) aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino); nitro; carboxy (e.g., HOOC—, alkoxycarbonyl, or alkylcarbonyloxy); acyl (e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl); cyano; halo; hydroxy; mercapto; sulfonyl (e.g., alkyl-S(O)₂— and aryl-S(O)₂—); sulfinyl (e.g., alkyl-S(O)—); sulfanyl (e.g., alkyl-S—); sulfoxy; urea; thiourea; sulfonamide; sulfamide; oxo; or carbamoyl.

As used herein, “cyclic moiety” includes cycloaliphatic, heterocycloaliphatic, aryl, or heteroaryl, each of which has been defined previously.

As used herein, the term “heterocycloaliphatic” encompasses a heterocycloalkyl group and a heterocycloalkenyl group, each of which being optionally substituted as set forth below.

As used herein, a “heterocycloalkyl” group refers to a 3-10 membered mono- or bicylic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl. A monocyclic heterocycloalkyl group can be fused with a phenyl moiety such as tetrahydroisoquinoline.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicycloheteroaliphatics are numbered according to standard chemical nomenclature.

A heterocycloalkyl or heterocycloalkenyl group can be optionally substituted with one or more substituents. Examples of such substitutents include, but are not limited to, aliphatic (e.g., alkyl, alkenyl, or alkynyl); arylaliphatic (e.g., arylalkyl), cycloaliphatic; (cycloaliphatic)aliphatic; heterocycloaliphatic; (heterocycloaliphatic)aliphatic; aryl; heteroaryl; heteroarylaliphatic (e.g., heteroarylalkyl); alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); azide (i.e., —N₃), nitro; carboxy (e.g., alkoxy-C(O)—); amido; amidoamino (e.g., —NR—C(O)—NRR′); thioamido (e.g., —C(S)—NRR′); thioamidoamino (e.g., —NR—C(S)—NRR′); alkoxyamido (e.g., —NR—C(O)-alkoxy or —C(O)—NR-alkoxy); acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl); sulfonyl (e.g., aliphatic-S(O)₂—, (aliphatic-0)-S(O)₂—O—, or amino-S(O)₂—); sulfonylamino (e.g., —NR—S(O)₂—OR′); sulfinyl (e.g., aliphatic-S(O)— or cycloaliphatic-S(O)—); sulfinylamino; sulfanyl (e.g., aliphatic-S—); cyano; halo; hydroxy; mercapto; sulfoxy; urea; thiourea; sulfonamide; sulfamide; carbamoyl; phosphinio (e.g., —P(O)(OR)R′); phosphonio (e.g., —O—P(O)(OR)R′); phosphinioamino (e.g., —NR—P(O)(OR′)R″); or phosphonioamino (e.g., —NR—P(O)(OR)(OR′)). Each of R, R′ and R″ in the just-mentioned examples can be independently an aliphatic.

A “heteroaryl” group, as used herein, refers to a monocyclic, bicyclic, or tricyclic ring system having 4 to 15 ring atoms wherein at least one of the ring atoms is a heteroatom (e.g., N, O, S, or combinations thereof and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4- to 8-membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, and 1,8-naphthyridyl.

Without limitation, examples of monocyclic heteroaryls include furyl, thiophenyl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, and 1,3,5-triazyl. Monocyclic heteroaryls are numbered according to standard chemical nomenclature.

Without limitation, examples of bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, and pteridyl. Bicyclic heteroaryls are numbered according to standard chemical nomenclature.

A heteroaryl is optionally substituted with one or more substituents. Examples of such substitutents include, but are not limited to, aliphatic (e.g., alkyl, alkenyl, or alkynyl); arylaliphatic (e.g., arylalkyl), cycloaliphatic; (cycloaliphatic)aliphatic; heterocycloaliphatic; (heterocycloaliphatic)aliphatic; aryl; heteroaryl; heteroarylaliphatic (e.g., heteroarylalkyl); alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); azide (i.e., —N₃), nitro; carboxy (e.g., alkoxy-C(O)—); amido; amidoamino (e.g., —NR—C(O)—NRR′); thioamido (e.g., —C(S)—NRR′); thioamidoamino (e.g., —NR—C(S)—NRR′); alkoxyamido (e.g., —NR—C(O)-alkoxy or —C(O)—NR-alkoxy); acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl); sulfonyl (e.g., aliphatic-S(O)₂—, (aliphatic-0)-S(O)₂—O—, or amino-S(O)₂—); sulfonylamino (e.g., —NR—S(O)₂—OR′); sulfinyl (e.g., aliphatic-S(O)— or cycloaliphatic-S(O)—); sulfinylamino; sulfanyl (e.g., aliphatic-S—); cyano; halo; hydroxy; mercapto; sulfoxy; urea; thiourea; sulfonamide; sulfamide; carbamoyl; phosphinio (e.g., —P(O)(OR)R′); phosphonio (e.g., —O—P(O)(OR)R′); phosphinioamino (e.g., —NR—P(O)(OR′)R″); or phosphonioamino (e.g., —NR—P(O)(OR)(OR′)). Each of R, R′ and R″ in the just-mentioned examples can be independently an aliphatic. Alternatively, a heteroaryl can be unsubstituted.

Non-limiting examples of substituted heteroaryls include (halo)heteroaryl (e.g., mono- and di-(halo)heteroaryl), (carboxy)heteroaryl (e.g., (alkoxycarbonyl)heteroaryl), cyanoheteroaryl, aminoheteroaryl (e.g., ((alkylsulfonyl)amino)heteroaryl and ((dialkyl)amino)heteroaryl), (amido)heteroaryl (e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, or ((alkylcarbonyl)amino)heteroaryl), (cyanoalkyl)heteroaryl, (alkoxy)heteroaryl, (sulfonamide)heteroaryl (e.g., (aminosulfonyl)heteroaryl), (sulfonyl)heteroaryl (e.g., (alkylsulfonyl)heteroaryl), (hydroxyalkyl)heteroaryl, (alkoxyalkyl)heteroaryl, (hydroxy)heteroaryl, ((carboxy)alkyl)heteroaryl, (((dialkyl)amino)alkyl)heteroaryl, (heterocycloaliphatic)heteroaryl, (cycloaliphatic)heteroaryl, (nitroalkyl)heteroaryl, (((alkylsulfonyl)amino)alkyl)heteroaryl, ((alkylsulfonyl)alkyl)heteroaryl, (cyanoalkyl)heteroaryl, (acyl)heteroaryl (e.g., (alkylcarbonyl)heteroaryl), (alkyl)heteroaryl, and (haloalkyl)heteroaryl (e.g., trihaloalkylheteroaryl).

A “heteroaraliphatic” group (e.g., a heteroaralkyl group) as used herein, refers to an aliphatic group (e.g., a C₁₋₄ alkyl group) that is substituted with a heteroaryl group. “Aliphatic,” “alkyl,” and “heteroaryl” have been defined above.

A “heteroaralkyl” group, as used herein, refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents. Examples of such substitutents include, but are not limited to, aliphatic (e.g., alkyl, alkenyl, or alkynyl); arylaliphatic (e.g., arylalkyl), cycloaliphatic; (cycloaliphatic)aliphatic; heterocycloaliphatic; (heterocycloaliphatic)aliphatic; aryl; heteroaryl; heteroarylaliphatic (e.g., heteroarylalkyl); alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); azide (i.e., —N₃), nitro; carboxy (e.g., alkoxy-C(O)—); amido; amidoamino (e.g., —NR—C(O)—NRR′); thioamido (e.g., —C(S)—NRR′); thioamidoamino (e.g., —NR—C(S)—NRR′); alkoxyamido (e.g., —NR—C(O)-alkoxy or —C(O)—NR-alkoxy); acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl); sulfonyl (e.g., aliphatic-S(O)₂—, (aliphatic-O)—S(O)₂—O—, or amino-S(O)₂—); sulfonylamino (e.g., —NR—S(O)₂—OR′); sulfinyl (e.g., aliphatic-S(O)— or cycloaliphatic-S(O)—); sulfinylamino; sulfanyl (e.g., aliphatic-S—); cyano; halo; hydroxy; mercapto; sulfoxy; urea; thiourea; sulfonamide; sulfamide; carbamoyl; phosphinio (e.g., —P(O)(OR)R′); phosphonio (e.g., —O—P(O)(OR)R′); phosphinioamino (e.g., —NR—P(O)(OR′)R″); or phosphonioamino (e.g., —NR—P(O)(OR)(OR′)). Each of R, R′ and R″ in the just-mentioned examples can be independently an aliphatic.

As used herein, an “acyl” group refers to a formyl group or R_{X}—C(O)— (such as -alkyl-C(O)—, also referred to as “alkylcarbonyl”) where R_{X} and “alkyl” have been defined previously. Acetyl and pivaloyl are examples of acyl groups.

As used herein, an “aroyl” or “heteroaroyl” group refers to an aryl-C(O)— or a heteroaryl-C(O)—. The aryl and heteroaryl portion of the aroyl or heteroaroyl is optionally substituted as previously defined.

As used herein, an “alkoxy” group refers to an alkyl-O— group wherein “alkyl” has been defined previously.

As used herein, a “carbamoyl” group refers to a group having the structure —O—C(O)—NR_{X}R_{Y} or —NR_{X}—C(O)—O—R_{Z}, wherein R_{X} and R_{Y} are as defined above and R_{Z} can be aliphatic, aryl, araliphatic, heterocycloaliphatic, heteroaryl, or heteroaraliphatic.

As used herein, a “carboxy” group refers to —COOH, —COOR_{X}, —OC(O)H, —OC(O)R_{X} when used as a terminal group; or —OC(O)— or —C(O)O— when used as an internal group.

As used herein, a “haloaliphatic” group refers to an aliphatic group substituted with 1-3 halogen atoms. For instance, the term haloalkyl includes the group —CF₃.

As used herein, a “mercapto” group refers to —SH.

As used herein, a “sulfonic” group refers to —S(O)₂OH or —S(O)₂OR_{X} when used terminally.

As used herein, a “sulfamide” group refers to the structure —NR_{X}—S(O)₂—NR_{Y}R_{Z} when used terminally and —NR_{X}—S(O)₂—NR_{Y}— when used internally, wherein R_{X}, R_{Y}, and R_{Z} have been defined above.

As used herein, a “sulfonamide” group refers to the structure —S(O)₂—NR_{X}R_{Y} or —NR_{X}—S(O)₂—R_{Z} when used terminally; or —S(O)₂—NR_{X}— or —NR_{X}—S(O)₂— when used internally, wherein Rx, Ry, and R_{Z} are defined above.

As used herein a “sulfanyl” group refers to —S—R_{X} when used terminally and —S— when used internally, wherein R_{X} has been defined above. Examples of sulfanyls include aliphatic-S—, cycloaliphatic-S—, aryl-S—, or the like.

As used herein a “sulfinyl” group refers to —S(O)—R_{X} when used terminally and —S(O)—when used internally, wherein RX has been defined above. Exemplary sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, or the like.

As used herein, a “sulfonyl” group refers to —S(O)₂—R_{X} when used terminally and —S(O)₂-when used internally, wherein R_{X} has been defined above. Exemplary sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂- or the like.

As used herein, a “sulfoxy” group refers to —O—SO—R_{X} or —SO—O—R_{X}, when used terminally and —O—S(O)— or —S(O)—O— when used internally, where RX has been defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refers to —C(O)—.

As used herein, an “oxo” refers to ═O.

As used herein, an “aminoalkyl” refers to the structure (R_{X})₂N-alkyl-.

As used herein, a “cyanoalkyl” refers to the structure (NC)-alkyl-.

As used herein, a “urea” group refers to the structure —NR_{X}—CO—NR_{Y}R_{Z} and a “thiourea” group refers to the structure —NR_{X}—CS—NR_{Y}R_{Z} when used terminally and —NRX—CO—NR_{Y}— or —NR_{X}—CS—NR_{Y}— when used internally, wherein R_{X}, R_{Y}, and R_{Z} have been defined above.

As used herein, a “guanidine” group refers to the structure —N═C(N(R_{X}R_{Y}))N(R_{X}R_{Y}) or —N(R_{X})C═(N(R_{X}))N(R_{X}R_{Y}), wherein R_{X} and R_{Y} have been defined above.

As used herein, the term “amidino” group refers to the structure —C═(NR_{X})N(R_{X}R_{Y}) wherein R_{X} and R_{Y} have been defined above.

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom:

The terms “terminally” and “internally” refer to the location of a group within a substituent. A group is terminal when the group is present at the end of the substituent not further bonded to the rest of the chemical structure. Carboxyalkyl, i.e., R_{X}O(O)C-alkyl, is an example of a carboxy group being used terminally. A group is internal when the group is present in the middle of a substituent to at the end of the substituent bound to the rest of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, the term “cyclic group” encompasses mono-, bi-, and tri-cyclic ring systems including cycloaliphatic, heterocycloaliphatic, aryl, or heteroaryl, each of which has been previously defined.

As used herein, the term “bridged bicyclic ring system” refers to a bicyclic heterocyclicalipahtic ring system or bicyclic cycloaliphatic ring system in which the rings have at least two common atoms. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxabicyclo[2.2.2]octyl, 1-azabicyclo[2.2.2]octyl, 3-azabicyclo[3.2.1]octyl, and 2,6-dioxatricyclo[3.3.1.03,7]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfonamide, sulfamide, oxo, or carbamoyl.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R₁, R₂, and R₃, and other variables contained therein Formula (I) encompass specific groups, such as alkyl and aryl. Unless otherwise noted, each of the specific groups for the variables R₁, R₂, and R₃, and other variables contained therein can be optionally substituted with one or more substituents described herein. Each substituent of a specific group is further optionally substituted with one to three of halo, cyano, oxoalkoxy, hydroxy, amino, nitro, aryl, haloalkyl, and alkyl. For instance, an alkyl group can be substituted with alkylsulfanyl and the alkylsulfanyl can be optionally substituted with one to three of halo, cyano, oxoalkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

The phrase “stable or chemically feasible,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, a “subject” for treatment generally refers and thus may be interchangeable with a “patient,” such as an animal (e.g., a mammal such as a human).

As used herein, an “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970).

Unless otherwise stated, the structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C— or ¹⁴C-enriched carbon are within the scope of this invention. Such compounds are useful, for example, as analytical tools or probes in biological assays.

### DETAILED DESCRIPTION OF THE INVENTION

In general, the invention features compounds of Formula (I), which modulate the function of one or more protein kinases.

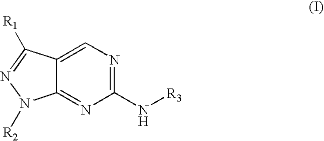

### Referring to Formula (I),

R¹ is hydrogen or halo;

R² is -L₁-Rₐ, wherein
L₁ is a bond and Rₐ is alkenyl, alkynyl, cycloalkyl, heterocycloalkyl, aryl, or heteroaryl; in which each of cycloalkyl, heterocycloalkyl, aryl, and heteroaryl is optionally substituted and has 8 to 16 ring atoms; or L₁ is alkyl and Rₐ is hydrogen, alkenyl, alkynyl, amino, hydroxy, alkoxycarbonyl, alkylcarbonyloxy, cycloalkyl, heterocycloalkyl, aryl, or heteroaryl; in which each of cycloalkyl, heterocycloalkyl, aryl, and heteroaryl is optionally substituted and has 8 to 16 ring atoms;

R³ is -R_{b}-L₂-R_{c}; wherein
R_{b} is aryl, heteroaryl, cycloalkyl, or heterocycloalkyl, and is optionally substituted with 1 to 3 substituents; wherein two of the substituents when adjacent, together with the atom or atoms to which they are attached, can form a 5- to 16-membered ring with 0 to 6 hetero ring atoms,
L₂ is a bond, —(CRₓR_{y})—, —N═, —O—, —S—, —SO—, —SO₂—, —CO—, —CO—O—, —O—CO—, —NRₓ—, —NRₓ—CO—, —NRₓ—SO₂—, —CO—NRₓ—, —SO₂—NRₓ—, —NRₓ—CO—O—, —NRₓ—SO₂—O—, —NRₓ—CO—NR_{y}—, —NRₓ—SO₂—NR_{y}—, —CO—NRₓ—NR_{y}—, —SO₂—NRₓ—NR_{y}—, —NRₓ—CO—CO—O—, —NRₓ—SO₂—SO₂—O—, —S(O)₂—Nₓ—CO—R_{y}—, —CO—N_{X}—S(O)₂—R_{y}—, or —(NRₓR_{y})C═N—O—;
R_{c} is hydrogen, alkyl, alkenyl, alkynyl, cycloalkyl, heterocycloalkyl, cycloalkenyl, heterocycloalkenyl, aryl, heteroaryl, cycloalkyl-alkyl, heterocycloalkyl-alkyl, cycloalkenyl-alkyl, heterocycloalkenyl-alkyl, aralkyl, or heteroaralkyl; and, except for hydrogen, is optionally substituted with 1 to 3 substituents;
each of Rₓ and R_{y}, independently, is hydrogen, hydroxy, alkyl, alkoxy, amino, —CO-alkyl, —CO-aryl, —SO₂-alkyl, —SO₂-aryl, —SO₂-heteroaryl, or —P(O)(O)-alkyl)₂, wherein the alkyl or aryl moiety in Rₓ or R_{y} is optionally substituted with 1 to 3 substituents; and
n is 0, 1, 2, or 3.

In Formula (I), R_{b} is optionally substituted with 1 to 3 substituents each independently selected from the group consisting of alkyl, alkenyl, alkynyl, cycloalkyl, heteroaryl, aryl, heteroaryl, arylalkyl, heteroarylalkyl, —OR, —SR, —NRR′, oxo, —C(O)—OR, —C(O)—NRR′, halo, —CN, —NO₂, —N₃, —C(O)R, —P(O)(OR)R′, —O—P(O)(OR)R′, —NR—P(O)(OR′)R″, —S(O)₂—(OR), —O—S(O)₂—(OR), —NR—S(O)₂—OR′, —NR—C(O)—OR′, —NR—C(O)—NR′R″, —NR—C(S)—NR′R″, —C(S)—NRR′, and thioalkyl; each of R and R′, independently, is hydrogen, alkyl, cycloalkyl, heterocycloalkyl, aryl, heteroaryl, aralkyl, or heteroaralkyl; and R″ is alkyl, cycloalkyl, heterocycloalkyl, aryl, heteroaryl, aralkyl, or heteroaralkyl.

R_{c} is, except when being hydrogen, optionally substituted with 1 to 3 substituents each independently selected from the group consisting alkyl, alkenyl, alkynyl, cycloalkyl, heteroaryl, aryl, heteroaryl, arylalkyl, heteroarylalkyl, —OR, —SR, —NRR′, oxo, —C(O)—OR, —C(O)—NRR′, halo, —CN, —NO₂, —N₃, —C(O)R, —P(O)(OR)R′, —O—P(O)(OR)R %-NR—P(O)(OR′)R″, —S(O)₂—(OR), —O—S(O)₂—(OR), —NR—S(O)₂—OR′, —NR—C(O)—OR′, —NR—C(O)—NR′R″, —NR—C(S)—NR′R″, —C(S)—NRR′, and thioalkyl; each of R and R′, independently, is hydrogen, alkyl, cycloalkyl, heterocycloalkyl, aryl, heteroaryl, aralkyl, or heteroaralkyl; and R″ is alkyl, cycloalkyl, heterocycloalkyl, aryl, heteroaryl, aralkyl, or heteroaralkyl.

The alkyl or aryl moiety in Rₓ and R_{y} is optionally substituted with 1 to 3 substituents each independently selected from the group consisting of alkyl, alkenyl, alkynyl, cycloalkyl, heteroaryl, aryl, heteroaryl, arylalkyl, heteroarylalkyl, —OR, —SR, —NRR′, oxo, —C(O)—OR, —C(O)—NRR′, halo, —CN, —NO₂, —N₃, —C(O)R, —P(O)(OR)R′, —O—P(O)(OR)R′, —NR—P(O)(OR′)R″, —S(O)₂—(OR), —O—S(O)₂—(OR), —NR—S(O)₂—OR′, —NR—C(O)—OR′, —NR—C(O)—NR′R″, —NR—C(S)—NR′R″, —C(S)—NRR′, and thioalkyl; each of R and R′, independently, is hydrogen, alkyl, cycloalkyl, heterocycloalkyl, aryl, heteroaryl, aralkyl, or heteroaralkyl; and R″ is alkyl, cycloalkyl, heterocycloalkyl, aryl, heteroaryl, aralkyl, or heteroaralkyl.

### Synthesis of Compounds of Formula (I)

Compounds of Formula (I) may be synthesized from commercially available or known starting materials by known methods. Exemplary synthetic routes to produce compounds of Formula (I) are provided in Schemes 1-5 below. The generic schemes are not limiting and can be applied to preparation of other compounds that include different variables than those explicitly shown below.

One method for preparing compounds of Formula (I) is illustrated in Scheme 1.

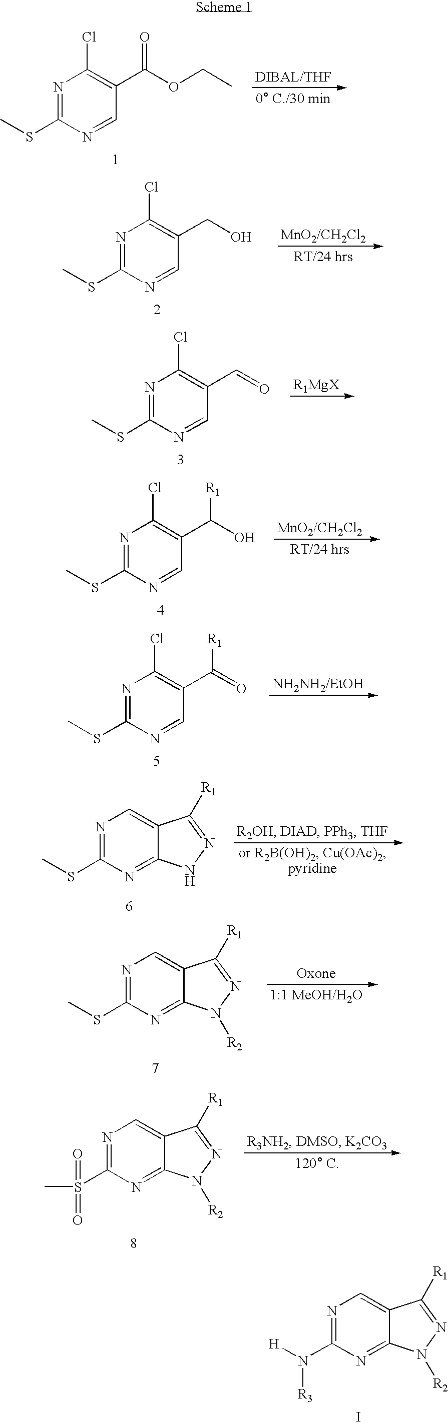

Referring to Scheme 1, the pyrimidine ester 1 is reduced to the corresponding alcohol 2 with, for example, diisobutylaluminum hydride (DIBAL). Oxidation of 2 with, for example, manganese dioxide provides the corresponding aldehyde 3. The aldehyde 3 may be reacted with hydrazine to provide compounds of formula 6 wherein R₁ is H. Alternatively, the aldehyde 3 may be reacted with, for example, a Grignard reagent R₁MgX to provide the intermediate alcohol 4 wherein R₁ is other than H. Oxidation of 4 provides a ketone 5 which may be reacted with hydrazine to provide the pyrazolopyrimidine 6. Alkylation of 6 with, for example, an alkyl halide such as R₂X provides the intermediate 7. Alternatively, treatment of 6 with, for example, an arylboronic acid such as R₂B(OH)₂ in presence of Cu(OAc)₂ provides the intermediate 7 (R₂=aryl). Oxidation of the thioether of 7 with, for example, Oxone® provides the sulfone 8. Reaction of 8 with an amine R₃NH₂ provides compounds of Formula (I).

Another method of preparing compounds of Formula (I), wherein R₁ is H, is illustrated in Scheme 2.

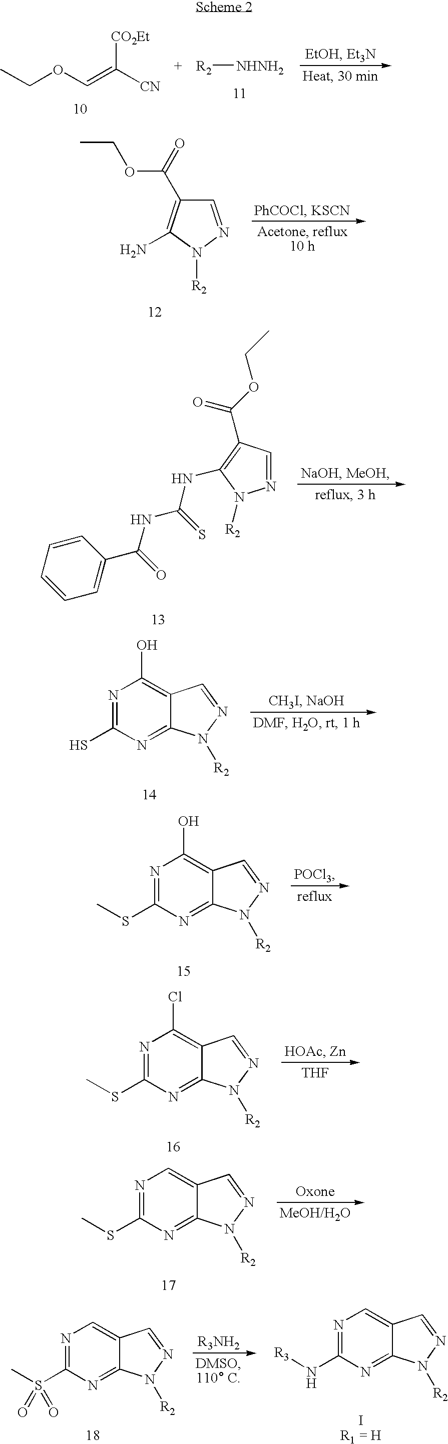

Referring to Scheme 2, reaction of the cyanoacrylate 10 with a hydrazine 11 provides the aminopyrazole 12. Reaction of the aminopyrazole 12 with benzoyl chloride and potassium thiocyanate provides the benzoylthio urea 13 which on treatment with sodium hydroxide in methanol provides a pyrrazolopyrimidine 14. Alkylation of the pyrrazolopyrimidine 14 with methyl iodied in the presence of sodium hydroxide provides a thioether intermediate 15. Reaction of the thioether intermediate 15 with phosphorousoxychloride provides a chloro pyrimidine 16 which is reduced to a pyrazolopyrimidine 17 with zinc in the presence of acetic acid. Oxidation of the pyrazolopyrimidine 17 with Oxone® provides a sulfone 18 which reacts with an amine R₃NH₂ to provide compounds of this invention, i.e., compounds of formula (I) in which R₁ is H.

In some embodiments where R₃ is, for example, an amino-substituted aryl, further examples of compounds of Formula (I) may be prepared as illustrated in Scheme 3.

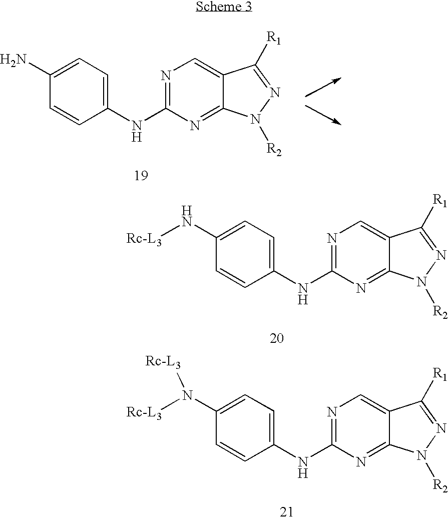

Referring to Scheme 3, an amino substituted compound 19 may be reacted with a compound of formula R_{c}-L₃-Q wherein L₃ is —C(O)—, —SO₂— or —P(OR^{x})₂— and Q represents a halide, or R_{c}-L₃-Q represents an acid anhydride, to provide monosubstituted compounds of formula 20 or disubstituted compounds of formula 21.

Another method of preparing compounds of formula I, wherein, for example, R₃ is an amino-substituted aryl, is illustrated in Scheme 4.

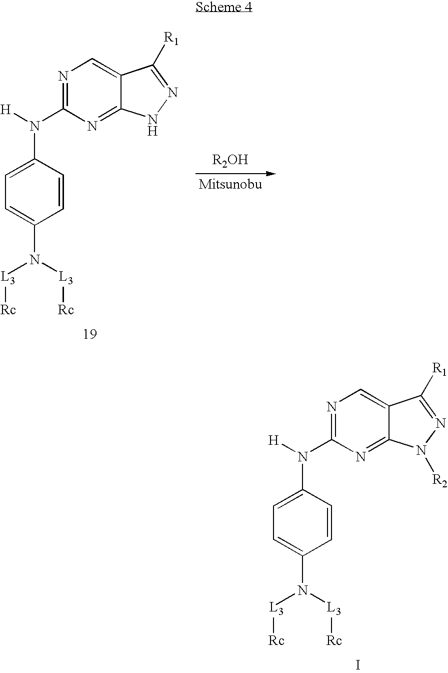

Referring to Scheme 4, the amino substituted compound 19 may be reacted with a compound of formula R₂OH to provide compounds of formula I.

In a further embodiment, compounds of Formula (I) wherein R₃ is an aryl-tetrazole may be prepared as illustrated in Scheme 5.

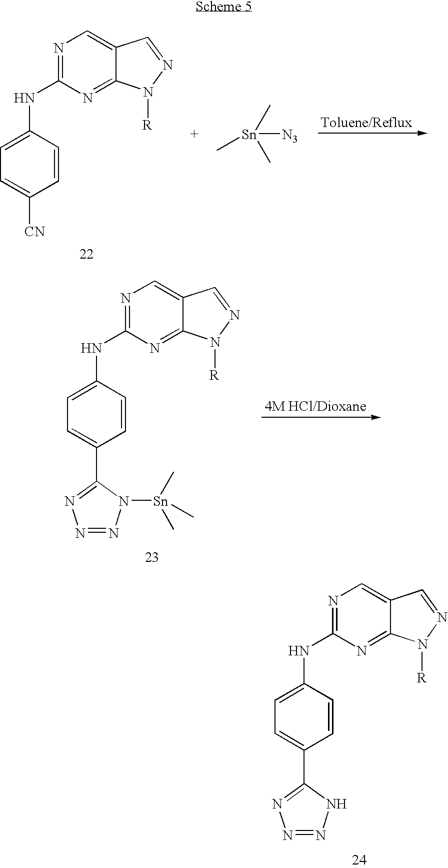

Referring to Scheme 5, a cyanosubstituted compound 22 is reacted with a trialkyltin azide, e.g. trimethyltin azide, to provide an intermediate tin-tetrazole 23. Hydrolysis of the compound 23 in the presence of a mineral acid, e.g. hydrochloric acid, provides compounds of formula 24.

### Administration of Compositions Containing Compounds of Formula (I)

As defined above, an effective amount is the amount required to confer a therapeutic effect on the treated patient. For a compound of Formula (I), an effective amount can range, for example, from about 1 mg/kg to about 150 mg/kg (e.g., from about 1 mg/kg to about 100 mg/kg). The effective amount may also vary, as recognized by those skilled in the art, dependant on route of administration, excipient usage, and the possibility of co-usage with other therapeutic treatments including use of other therapeutic agents and/or radiation therapy.

The amount of the compounds of the present invention that may be combined with the carrier materials to produce a composition in a single dosage form will vary depending upon the host treated, the particular mode of administration. For instance, the compositions may be formulated so that a dosage of between 0.01-100 mg/kg body weight/day of the modulator can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Depending upon the particular condition, or disease, to be treated or prevented, additional therapeutic agents, which are normally administered to treat or prevent that condition, may also be present in the compositions of this invention. As used herein, additional therapeutic agents that are normally administered to treat or prevent a particular disease, or condition, are known as “appropriate for the disease, or condition, being treated.”

Compounds of Formula (I) can be administered in any manner suitable for the administration of pharmaceutical compounds, including, but not limited to, pills, tablets, capsules, aerosols, suppositories, liquid formulations for ingestion or injection or for use as eye or ear drops, dietary supplements, and topical preparations. The pharmaceutically acceptable compositions include aqueous solutions of the active agent, in an isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the therapeutic compounds. As to route of administration, the compositions can be administered orally, intranasally, transdermally, intradermally, vaginally, intraaurally, intraocularly, buccally, rectally, transmucosally, or via inhalation, implantation (e.g., surgically), or intravenous administration. The compositions can be administered to an animal (e.g., a mammal such as a human, non-human primate, horse, dog, cow, pig, sheep, goat, cat, mouse, rat, guinea pig, rabbit, hamster, gerbil, or ferret, or a bird, or a reptile such as a lizard).

Controlled release of therapeutic agents can utilize various technologies. Devices are known having a monolithic layer or coating incorporating a heterogeneous solution and/or dispersion of an active agent in a polymeric substance, where the diffusion of the agent is rate limiting, as the agent diffuses through the polymer to the polymer-fluid interface and is released into the surrounding fluid. In some devices, a soluble substance is also dissolved or dispersed in the polymeric material, such that additional pores or channels are left after the material dissolves. A matrix device is generally diffusion limited as well, but with the channels or other internal geometry of the device also playing a role in releasing the agent to the fluid. The channels can be pre-existing channels or channels left behind by released agent or other soluble substances.

Erodible or degradable devices typically have the active agent physically immobilized in the polymer. The active agent can be dissolved and/or dispersed throughout the polymeric material. The polymeric material is often hydrolytically degraded over time through hydrolysis of labile bonds, allowing the polymer to erode into the fluid, releasing the active agent into the fluid. Hydrophilic polymers have a generally faster rate of erosion relative to hydrophobic polymers. Hydrophobic polymers are believed to have almost purely surface diffusion of active agent, having erosion from the surface inwards. Hydrophilic polymers are believed to allow water to penetrate the surface of the polymer, allowing hydrolysis of labile bonds beneath the surface, which can lead to homogeneous or bulk erosion of polymer.

The implantable device coating can include a blend of polymers each having a different release rate of the therapeutic agent. For instance, the coating can include a polylactic acid/polyethylene oxide (PLA-PEO) copolymer and a polylactic acid/polycaprolactone (PLA-PCL) copolymer. The polylactic acid/polyethylene oxide (PLA-PEO) copolymer can exhibit a higher release rate of therapeutic agent relative to the polylactic acid/polycaprolactone (PLA-PCL) copolymer. The relative amounts and dosage rates of therapeutic agent delivered over time can be controlled by controlling the relative amounts of the faster releasing polymers relative to the slower releasing polymers. For higher initial release rates the proportion of faster releasing polymer can be increased relative to the slower releasing polymer. If most of the dosage is desired to be released over a long time period, most of the polymer can be the slower releasing polymer. The stent can be coated by spraying the stent with a solution or dispersion of polymer, active agent, and solvent. The solvent can be evaporated, leaving a coating of polymer and active agent. The active agent can be dissolved and/or dispersed in the polymer. In some embodiments, the co-polymers can be extruded over the stent body.

### Uses of Compounds of Formula (I)

The compounds of this invention have inhibitory effect on protein kinases such as one or more of multiple mitotic kinases (e.g., Aurora kinase, polo-like kinase, or cyclin-dependent kinase) as found in cell mitosis, e.g., abnormal growth of cells of proliferation of tumor cells.

Accordingly, the compounds of this invention can be used for inhibiting the abnormal growth of cells, including transformed cells, by administering an effective amount of a compound of the invention. Abnormal growth of cells refers to cell growth independent of normal regulatory mechanisms (e.g. loss of contact inhibition). This includes the abnormal growth of: (1) tumor cells (tumors) expressing an activated ras oncogene; (2) tumor cells in which the ras protein is activated as a result of oncogenic mutation of another gene; (3) benign and malignant cells of other proliferative diseases in which aberrant ras activation occurs. Furthermore, it has been suggested in literature that ras oncogenes not only contribute to the growth of tumors in vivo by a direct effect on tumor cell growth but also indirectly, i.e. by facilitating tumor-induced angiogenesis (see Rak. J. et al, Cancer Research, 55: 4575-4580, 1995). Hence, pharmacologically targeting mutant ras oncogenes could conceivably suppress solid tumor growth in vivo, in part, by inhibiting tumor-induced angiogenesis.

Examples of tumors which may be inhibited by the compounds of this invention include, but are not limited to, lung cancer (e.g. adenocarcinoma), pancreatic cancers (e.g. pancreatic carcinoma such as, for example exocrine pancreatic carcinoma), colon cancers (e.g. colorectal carcinomas, such as, for example, colon adenocarcinoma and colon adenoma), hematopoietic tumors of lymphoid lineage (e.g. acute lymphocytic leukemia, B-cell lymphoma, Burkitt's lymphoma), myeloid leukemias (for example, acute myelogenous leukemia (AML)), thyroid follicular cancer, myelodysplastic syndrome (MDS), tumors of mesenchymal origin (e.g. fibrosarcomas and rhabdomyosarcomas), melanomas, teratocarcinomas, neuroblastomas, gliomas, benign tumor of the skin (e.g. keratoacanthomas), breast carcinoma, kidney carcinoma, ovary carcinoma, bladder carcinoma and epidermal carcinoma. The compound can be administered in a suitable manner, e.g., intravenously, subcutenously, orally, parenterally, or topically.

This invention may also provide a method for inhibiting proliferative diseases, both benign and malignant, wherein ras proteins are aberrantly activated as a result of oncogenic mutation in genes. With said inhibition being accomplished by the administration of an effective amount of the compounds described herein, to a subject in need of such a treatment. For example, the benign proliferative disorder neuro-fibromatosis, or tumors in which ras is activated due to mutation or overexpression of tyrosine kinase oncogenes, may be inhibited by the compounds of this invention.

All references cited within this document are incorporated herein in their entirety by reference.

The following examples are set forth to enable the invention described herein being more readily understood. These examples are for illustrative purposes only and are not to be construed as limiting this invention in any manner.

### GENERAL EXPERIMENTAL

¹H-NMR spectra were determined on a Bruker 400 MHz instrument. Mass spectra were recorded on an Agilent ESI-TOF mass spectrometer. HPLC was performed on Agilent 1100 instruments. The HPLC method used for these compounds is as follows:

Column: Agilent Zorbax 300 SB C18, 4.6×150 mm, 5 μm;

Column Temperature: ambient;

Flow Rate: 1.0 ml/min,

Gradient: 5% acetonitrile (0.05 TFA) in water (0.1% TFA) to 100% acetonitrile (0.05% TFA) in 7 minutes, hold at 100% for 2 minutes.

### General Procedures to Prepare Pyrazolo[3,4-d]pyrimidines

### General Procedure 1-1: Synthesis of 1-alkyl-6-(methylthio)-1H-pyrazolo[3,4-d]pyrimidine

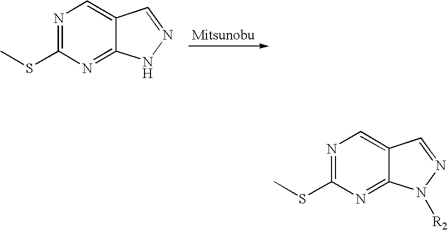

Synthesis of this compound was conducted according to procedures described by J. Adams in WO03/029209A2. Specifically, to a solution of triphenyl phosphine in THF at −78° C. under nitrogen, diisopropylazodicarboxylate (DIAD) was added dropwise. The reaction mixture was stirred for five minutes before an alcohol R₂OH(R₂ is alkyl) was added. The mixture was stirred for another 5 minutes then 6-(methylthio)-1H-pyrazolo[3,4-d]pyrimidine was added. The resultant reaction mixture was stirred at room temperature for 2 hours. The solvent was rotavaped, diethyl ether was added, the mixture was filtered and the filtrate was rotavaped. The crude material was purified by flash chromatography (EtOAc/Hexane) to provide the title compound.

### General Procedure 1-2: Synthesis of 1-cycloheptyl-6-(methylthio)-1H-pyrazolo[3,4-d]pyrimidine

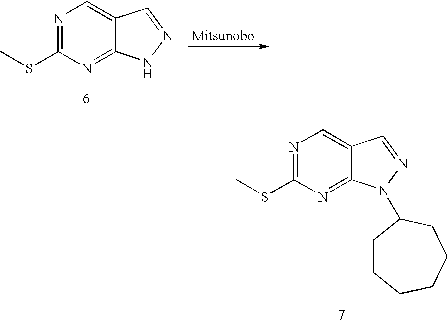

To a solution of triphenyl phosphine (3.05 g, 11.6 mmol) in 20 mL THF at −78° C. under nitrogen, diisopropylazodicarboxylate (DIAD) (2.36 g, 11.6 mmol) was added dropwise. The reaction mixture was allowed to stir for five minutes before cycloheptanol (1.34 g, 11.7 mmol) was added. The mixture was stirred for 5 minutes then 6-(methylthio)-1H-pyrazolo[3,4-d]pyrimidine (1.6 g, 9.6 mmol) was added. The reaction mixture was stirred at RT for 2 hours. The mixture was chromatographed over silica gel (10% EtOAc/Hexanes) to provide 2.32 g solid (90.6% yield) of 1-cycloheptyl-6-(methylthio)-1H-pyrazolo[3,4-d]pyrimidine.

Rf (25% EtOAc/Hexanes): 0.5.

HPLC tR: 7.69 minutes.

### General Procedure 1-3: Synthesis of 1-Aryl-6-(methylthio)-1H-pyrazolo[3,4-d]pyrimidine

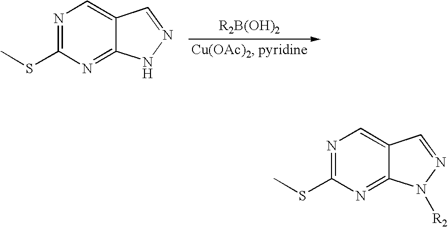

Synthesis of this compound was conducted according to procedures described by P. Y. S. Lam in Tetrahedron Lett., 1998, 39, 2941. Specifically, to activated molecular serive MS4 Å were added DCM, copper acetate, and pyridine. After the mixture was stirred for 15 minutes at the room temperature, 6-(methylthio)-1H-pyrazolo[3,4-d]pyrimidine and R₂B(OH)₂ (R₂ is aryl) were added to the mixture. After stirring for 2 days at the room temperature, the reaction mixture was filtered, washed with a mixture of water, Brine, and ammonium hydroxide, and then dried over sodium sulfate. The crude material thus obtained was purified by flash chromatography (Ethyl acetate or acetone/hexane) to give the title compound.

### General Procedure 1-4: Synthesis of ethyl 5-amino-1-substituted-1H-pyrazole-4-carboxylates

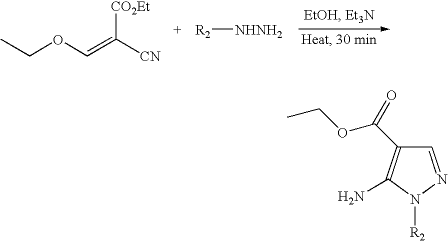

The synthesis of this compound was performed according to procedures described by M. Kopp in J. Heterocyclic Chem., 2001, 38, 1045-1050. Specifically, to a round-bottom flask was added an optionally substituted hydrazine hydrochloride in ethanol and triethylamine. The mixture was stirred for 15 minutes at the room temperature before being it was heated at 90° C. and (Z)-2-cyano-3-ethoxycarboxylic acid ethyl ester was added. The reaction mixture thus obtained was heated for 1 hour at 90° C. and then cooled to the room temperature. The precipitate was filtered and washed with ether, and then dried over high vacuum to give the title compound.

### General Procedure 1-5: Synthesis of ethyl 5-(3-benzoylthioureido)-1-substituted-1H-pyrazole-4-carboxylate

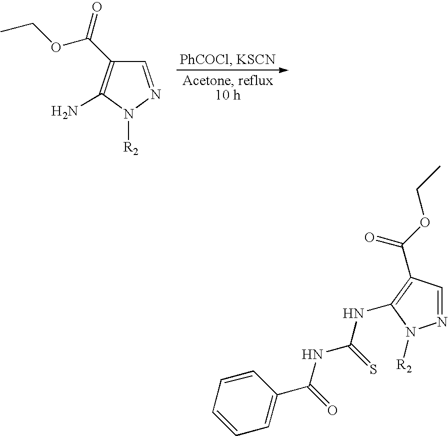

The title compound was synthesized according to procedures described by F. Carraro et al. in J. Med. Chem., 2006, 49, 1549-1561. Specifically, to a round bottom flask was added potassium thiocyanate in acetone followed by benzoyl chloride at the room temperature. The reaction mixture was stirred for 10 minutes before ethyl 5-amino-1-substituted-1H-pyrazole-4-carboxylate was added to the mixture. The resultant mixture was heated to reflux overnight and cooled to the room temperature. The solvent was removed by rotary evaporation. The residue was extracted with ethyl acetate and washed with water, brine, dried over MgSO₄, and concentrated to give the title compound.

### General Procedure 1-6: Synthesis of 6-mercapto-1-substituted-1H-pyrazolo[3,4-d]pyrimidin-4-ol

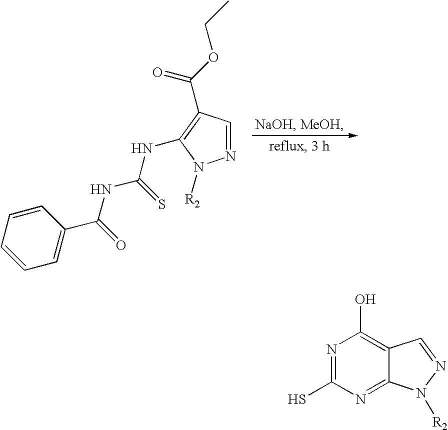

Synthesis of the title compound was conducted according to procedures described by F. Carraro et al. in J. Med. Chem., 2006, 49, 1549-1561. Specifically, to ethyl 5-(3-benzoylthioureido)-1-substituted-1H-pyrazole-4-carboxylate in methanol was added sodium hydroxide in water at the room temperature and the resultant reaction mixture was heated under reflux for 3 hours. The reaction mixture was then cooled to the room temperature and acidified with saturated NH₄Cl. The resultant precipitate was filtered to give the title compound.

### General Procedure 1-7: Synthesis of 1-substituted-6-(methylthio)-1H-pyrazolo[3,4-d]pyrimidin-4-ol

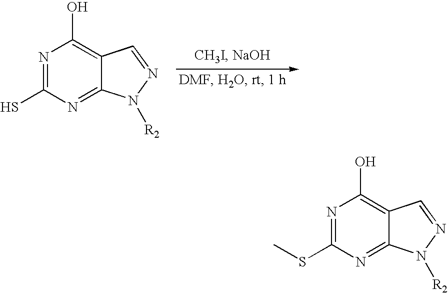

Synthesis of the title compound was conducted according to procedures described by F. Carraro et al. in the Journal of Medicinal Chemistry, 2006, 49, 1549-1561. Specifically, to 6-mercapto-1-substituted-1H-pyrazolo[3,4-d]pyrimidin-4-ol in DMF was added NaOH in water and an alkyl iodide (e.g. methyl iodide) at room temperature. The reaction mixture was stirred for 1 hour and neutralized with saturated NH₄Cl. The resultant precipitate was filtered to give the title compound.

### General Procedure 1-8: Synthesis of 4-chloro-1substituted-6-(methylthio)-1H-pyrazolo[3,4-d]pyrimidine

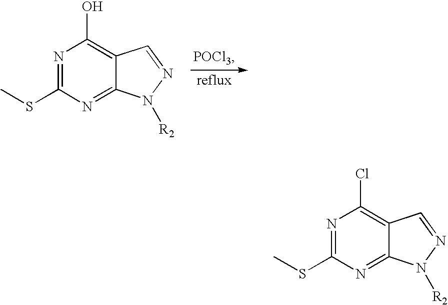

Synthesis of the title compound was conducted according to procedures described by F. Carraro et al. in the Journal of Medicinal Chemistry, 2006, 49, 1549-1561. Specifically, to 1-substituted-6-(methylthio)-1H-pyrazolo[3,4-d]pyrimidin-4-ol was added POCl₃ at the room temperature and the resultant mixture was heated under reflux for 1 hour. The reaction mixture was cooled to the room temperature and a precipitate was formed and filtered to give the title compound.

### General Procedure 1-9: Synthesis of 6-(methylthio)-1-substituted-1H-pyrazolo[3,4-d]pyrimidine

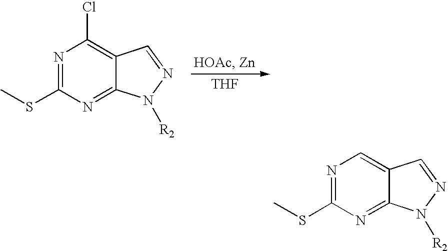

To 4-chloro-1substituted-6-(methylthio)-1H-pyrazolo[3,4-d]pyrimidine in THF and acetic acid was added Rieke zinc at the room temperature. The reaction mixture was heated at 80° C. for 20 minutes and then cooled to the room temperature. The cooled reaction mixture was then filtered through celite and the filtrate was concentrated and extracted with EtOAc, washed with water, brine, dried, and concentrated to give a crude product. The crude material was purified by flash chromatography (EtOAc/Hexane) to give the title compound.

### General Procedure 1-10: Synthesis of 3-optionally substituted-6-(methylsulfonyl)-1-substituted-1H-pyrazolo[3,4-d]pyrimidine

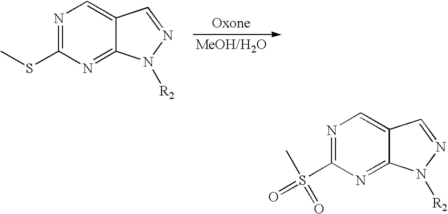

To 6-(methylthio)-1-substituted-1H-pyrazolo[3,4-d]pyrimidine in methanol was added Oxone® (5 eq.) in water and the mixture stirred at the room temperature overnight. The reaction mixture was concentrated and then extracted with EtOAc, washed with water, brine, dried, and concentrated. The crude material was purified by flash chromatography (Ethyl acetate/Hexane) to give the title compound.

### General Procedure 1-11: Synthesis of compounds of Formula (I)

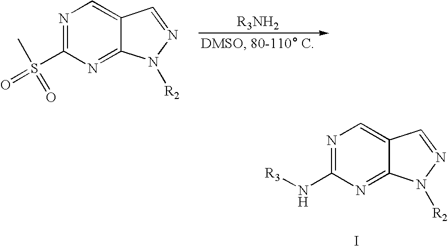

To a sealed tube was added 1-substituted-6-(methylsulfonyl)-1H-pyrazolo[3,4-d]pyrimidine and optionally substituted aniline (5 eq.) in DMSO at the room temperature. The reaction mixture was heated to about 110° C. from 30 minutes to overnight and then cooled to the room temperature. The mixture was quenched with water and extracted with EtOAc, washed with water, brine, dried, and concentrated to give a crude product. The crude material was purified by flash chromatography (Ethyl acetate/Hexane or DCM/MeOH) to give the compounds of Formula (I).

### General Procedure 1-12: Synthesis of N-(4-(1-substituted-1H-pyrazolo[3,4-d]pyrimidin-6-ylamino)phenyl)methanesulfonamide

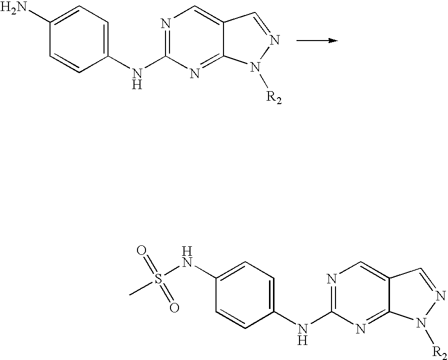

To N1-(1-substituted-1H-pyrazolo[3,4-d]pyrimidin-6-yl)benzene-1,4-diamine in CHCl₃ was added methanesulfonyl chloride (1 eq.) or methanesulfonic anhydride and DIEA (1.5 eq.) at the room temperature. The reaction mixture was stirred for 15 minutes at the room temperature and then purified by flash chromatography (Ethyl acetate/Hexane or DCM/MeOH) to give the title compound.

### General Procedure 1-13: Synthesis of N-(4-(1-substituted-1H-pyrazolo[3,4-d]pyrimidin-6-ylamino)phenyl)-N-(methylsulfonyl)methanesulfonamide

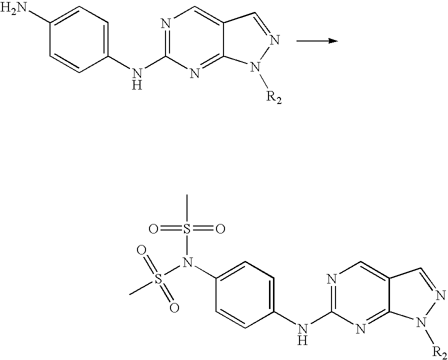

To N¹-(1-substituted-1H-pyrazolo[3,4-d]pyrimidin-6-yl)benzene-1,4-diamine in CHCl₃ was added methanesulfonyl chloride (5 eq.) or methanesulfonic anhydride and DIEA (7.5 eq.) at the room temperature. The reaction mixture was stirred for 15 minutes at the room temperature and then purified by flash chromatography (Ethyl acetate/Hexane or DCM/MeOH) to give the title compound.

### General Procedure 1-14: Synthesis of 4-(1-cycloheptyl-1H-pyrazolo[3,4-d]pyrimidin-6-ylamino)-N-(substituted-sulfonyl)benzamide

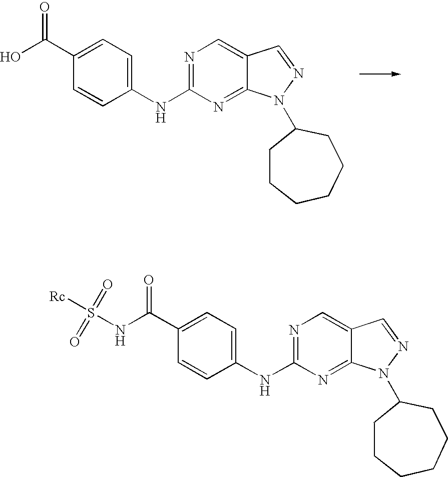

The synthesis of the title compound was conducted according to procedures described by C. F. Sturino et al. in Tetrahedron Lett., 1998, 39, 5891. Specifically, to 4-(1-cycloheptyl-1H-pyrazolo[3,4-d]pyrimidin-6-ylamino)benzoic acid in a 1/1/1/mixture of t-BuOH/1,2-dichloroethane/DMF were added N-(3-Dimethylaminopropyl)-N′-ethylcarbodiimide hydrochloride (Seq.), DMAP (10 eq.) and S-substituted-sulfonamide (10 eq). After the reaction mixture was stirred for 2 hours at the room temperature, 2.0 M HCl was added to it. The resultant mixture was extracted with EtOAc, washed with water, dried over sodium sulfate, and concentrated under reduce pressure to give a crude product. After trituration of the crude in DCM, the title compound was isolated as a white solid.

### General Procedure 1-15: Synthesis of N-(4-(1H-tetrazol-5-yl)phenyl)-1-substituted-1H-pyrazolo[3,4-d]pyrimidin-6-amine

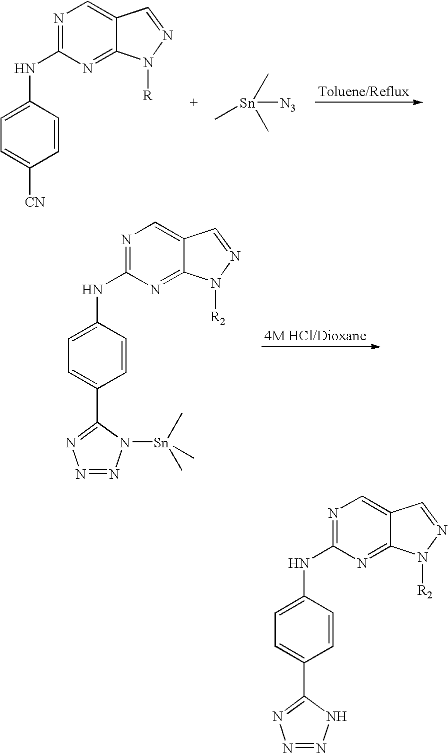

Synthesis of the title compound was conducted according to procedures described by J. V. Duncia in J. Org. Chem., 1991, 56, 2395-2400. Specifically, a suspension of 4-(1-substituted-1H-pyrazolo[3,4-d]pyrimidin-6-ylamino)benzonitrile (1.0 mmol) and trimethyltin azide (2.00 mmol) were heated under reflux in 5 mL toluene for 40-60 hours under the nitrogen atmosphere, which gave a crude trimethyltin intermediate. The crude intermediate was dissolved into 5 mL of dioxane and was stirred for 30 minutes in the presence of 4.0 M HCl to give the title compound.

### General Procedures to Prepare 1H-indol-4-ylboronic acid and 5,6,7,8-tetrahydronaphthalen-1-ylboronic acid

### General Procedure 2-1: Synthesis of 7-substituted-1H-indol-4-yl-boronic acids

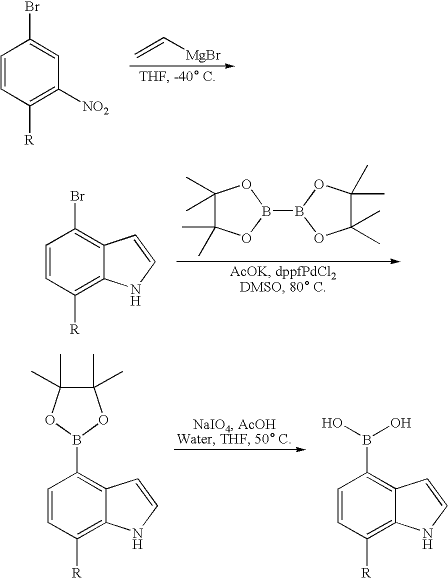

Synthesis of the title compound was conducted according to procedures described by L. Li in Tetrahedron Lett., 2003, 44, 5987-5990. Specifically, a solution of 6-substituted-3-bromo-nitrobenzene was treated with vinylmagnesium bromide to give a 7-substituted indole which was purified by flash chromatography. The 7-substituted indole then reacted with bis (pinacolato)diboron in presence of palladium (0) to afford a 7-substituted-4-(4,4,5,5-tetramethyl-1,3,2-dioxaborolan-2-yl)-1H-indole which was hydrolyzed into the title compound using sodium metaperiodate in Acetic/water/THF.

### General Procedure 2-2: Synthesis of 4-(benzyloxy)-5,6,7,8-tetrahydronaphthalen-1-ylboronic acid

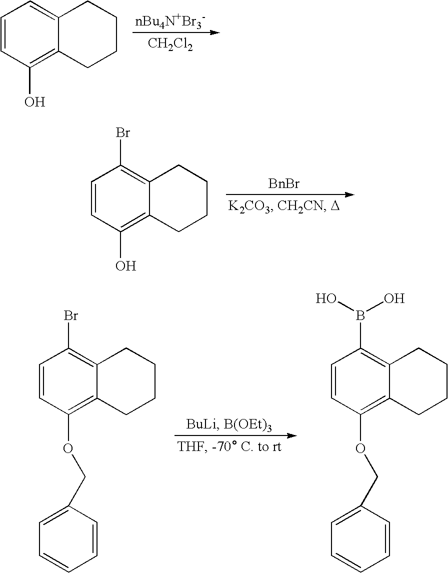

Synthesis of the title compound was conducted according to procedures described by John A. Lowe, III, in J. Med. Chem., 2004, 47, 1575-1586. Specifically, 4-bromo-1-naphthol was prepared from 1-napthol by treating with 1 equivalent of tributylammonium tribromide. It was then dissolved in acetonitrile, treated with benzyl bromide and potassium carbonate, and heated under reflux for 14 hours to provide 4-bromo-1-benzyloxynaphthalene. The compound 4-bromo-1-benzyloxynaphthalene then reacted with n-butyllithium and triethyl borate to afford the title compound.

### General Procedures to Prepare 3,4-Substituted Anilines

### General Procedure 3-1: Synthesis of (4-((4-methylpiperazin-1-yl)methyl)aniline)

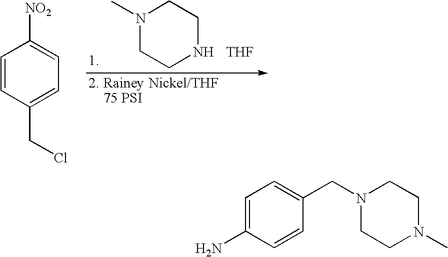

The synthesis of the title compound was conducted as described in U.S. Pat. Appl. Publ. No. 2006058341 (Mar. 16, 2006). Specifically, to a solution of 4-nitrobenzyl chloride in THF at the room temperature was added 1-methyl piperazine. The solution was stirred for 3 hours after which time the crude reaction was diluted with ethyl acetate and washed repeatedly with water. The dried organics were concentrated to give directly the 4-nitrobenzylamine adduct. This was subsequently treated with Rainey Nickel in THF at 75PSI for 12 hours to give the title compound.

### General Procedure 3-2: Synthesis of ethyl 2-(4-aminophenyl)-2-methylpropanoate analogues

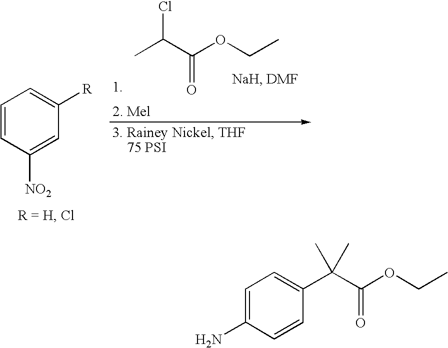

The synthesis of the title compound was conducted according to procedures described by Lawrence et al. in J. Org. Chem., 2002, 67, 457-464. To a suspension of sodium hydride in DMF at 0° C. was added dropwise a premixed solution of ethyl-2-chloropropionate and nitrobenzene in DMF. This was stirred at 0° C. for 30 minutes then warmed to 25° C. To this solution was then added methyl iodide and the mixture was stirred for 2 more hours. The reaction solution was quenched with 1 M HCl and diluted with methylene chloride. The organics were washed with saturated aqueous sodium bicarbonate solution and dried over sodium sulfate. The concentrated organics gave the title compound as a viscous brown oil without need for purification. When R is chlorine, flash chromatography is necessary.

### General Procedure 3-3: Synthesis of ethyl 2-(4-amino-2-chlorophenyl)acetate

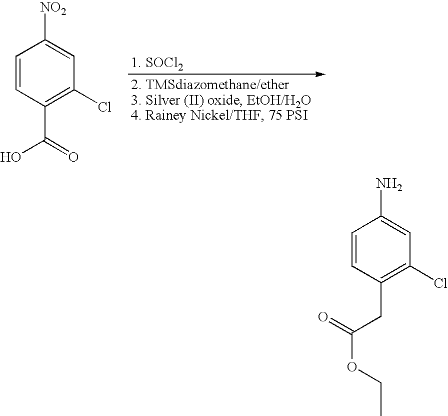

The synthesis of the title compound was conducted according to procedures described by G. Nannini in Arzneimittel-Forschung, 1973, 23, 1090-1100. Specifically, 2-chloro-4-nitrobenzoic acid was taken up into neat thionyl chloride and refluxed for 1 hour. The solvent was removed under reduced pressure to give 2-chloro-4-nitrobenzoyl chloride as a yellow oil. This was treated with trimethylsilyldiazomethane in diethyl ether at the room temperature. The resulting amber oil was purified via flash chromatography to give the dizaoketone as a crystalline yellow solid. This was taken up into a wet ethanol solution and treated with an aqueous slurry of silver(II) oxide. After filtering off the catalyst the mother liquor was diluted with water and extracted with ethyl acetate. Drying of the organics with sodium sulfate followed by concentration afforded the title compound as a viscous yellow oil without need for chromatography.

### General procedure 3-4: Synthesis of 4-(methylsulfonylmethyl)anline analogues

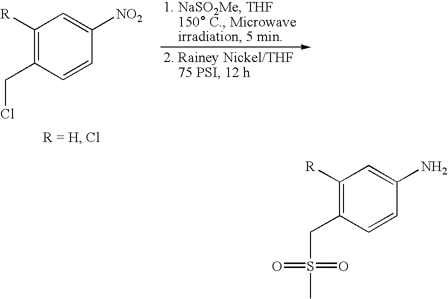

The synthesis of the title compound was conducted according to procedures described by G. Huiping in Bioorganic & Medicinal Chemistry Letters, 2004, 14, 187-190. Specifically, a 4-nitro-benzylchloride was taken up into THF to which was added sodium methylsulfinate. The reaction was irradiated with microwaves for 5 minutes at 150° C. The crude reaction solution was diluted with water and extracted with ethyl acetate. The dried organics were concentrated to give the displacement adduct as a tan solid. This latter material was treated with Rainey Nickel in THF at 75 PSI for 12 hours to give the title compound as a tan solid.

### General Procedure to Prepare 1-(4-Aminophenyl)-2-(dialkylamino)ethanol and 143-Aminophenyl)-2-(dialkylamino)ethanol

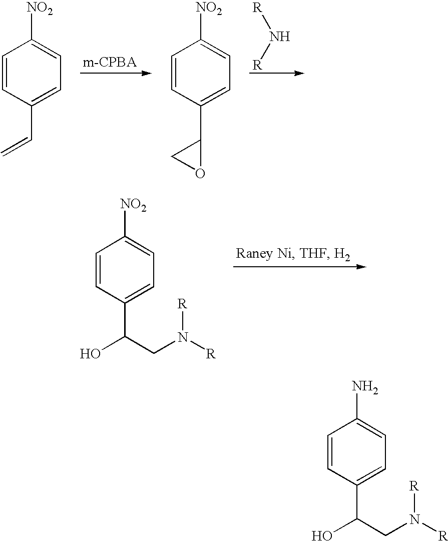

### Step 1: Synthesis 2-(4-nitrophenyl)oxirane

Synthesis of the title compound was conducted according to procedures described by K. Takai in Angewandte Chemie, 1981, 93 (8), 707. A solution of p-Nitrostyrene (1.40 g, 9.4 mmol.) and chloroperbenzoic acid (2.50 g, 11.2 mmol.) in chloroform (21.0 ml) was refluxed in an oil bath for 4 hrs., monitored by TLC (40% ethyl acetate in hexanes solution). Reaction mixture was cooled down to room temperature and filtered. The white precipitate was discarded and the remaining solution was concentrated, absorbed on 3.0 gram silica gel and purified via flash chromatography, eluted with 25% ethyl acetate in hexanes solutions to give the title compound, 2-(4-nitro-phenyl)-oxirane, in 43% yield (2.20 g).

### Step 2: Synthesis of 1-(4-nitrophenyl)-2-(piperidin-1-yl)ethanol

Synthesis of the title compound was conducted according to procedures described by U. M. Teotino in Farmaco, Edizione Scientifica 1962, 17, 252-65. A solution of 2-(4-nitrophenyl)-oxirane (0.80 g, 3.0 mmol.) and piperidine (1.00 ml, 10.1 mmol.) in absolute ethanol (100.0 ml) was refluxed in an oil bath for 4 hrs., monitored by TLC (5% methanol in dichloromethane solutions). Reaction solution was cooled down to room temperature, absorbed on 1.50 gram silica gel and purified via flash chromatography, eluted with 2% and 4% methanol in dichloromethane solutions to give the title compound, 1-(4-nitro-phenyl)-2-piperidin-1-yl-ethanol, in 86% yield (0.65 g).

### Step 3: Synthesis of 1-(4-aminophenyl)-2-(piperidin-1-yl)ethanol

To a solution of 1-(4-nitro-phenyl)-2-piperidin-1-yl-ethanol (0.60 g, 2.4 mmol.) in tetrahydrofuran (25.0 ml), slurry solution of raney-nickel in water (2.5 ml) was added and hydrogenation was carried on at 75 psi for 12 hrs. The residue was filtered cautiously through celite and then evaporated to dryness to give the title compound, 1-(4-amino-phenyl)-2-piperidin-1-yl-ethanol in 90% yield (0.47 g).

### General Procedures to Prepare Other Intermediates

### General Procedure 5-1: (4-Chloro-2-(methylthio)pyrimidin-5-yl)methanol

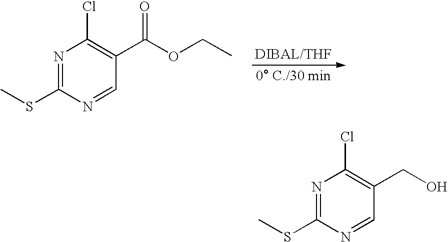

To a solution of ethyl 4-chloro-2-(methylthio)pyrimidine-5-carboxylate (10.7 g, 46 mmol), in dry THF (50 mL), on an ice-bath, DIBAL (185 mL, 1 M in THF, Aldrich Cat. No. 214981, 185 mmol) was added dropwise over 30 minutes (via pressure equalizing dropping funnel) under the nitrogen atmosphere. The resulting yellow transparent solution was stirred for additional 30 minutes before being transferred to a dry 1 L Erlenmeyer flask. On an ice bath, the reaction mixture was then quenched carefully, added dropwise with saturated solution of Na₂SO₄ to give a yellow warm gel-like solid mixture. To this solid mixture, EtOAc (200 mL) was added, followed by dropwise addition of 6.0 N aqueous HCl to dissolve the solid into an aqueous solution of pH 3-4. The EtOAc layer was collected. The aqueous layer was again extracted twice with EtOAc (twice, 200 mL each). The combined EtOAc layers were washed with water (twice, 200 mL each time), dried with MgSO₄, and concentrated to give an off-white solid which was suspended in 75 mL petroleum ether and refluxed for 10 minutes. The mixture was cooled to the ambient temperature and then filtered to give the title compound (4.67 g, 53%, HPLC: 97% pure).

HPLC Rt: 4.72 min.

¹H-NMR (CDCl₃): δ 8.54 (s, 1H), 4.74 (d, 2H), 2.57 (s, 3H), 2.10 (t, 1H).

### General Procedure 5-2: 4-Chloro-2-(methylthio)pyrimidine-5-carbaldehyde

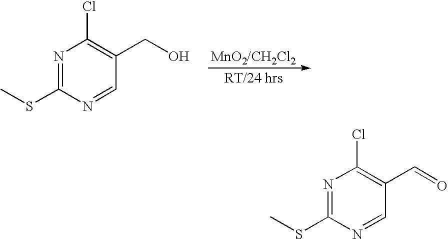

A mixture of (4-chloro-2-(methylthio)pyrimidin-5-yl)methanol (9.0 g, 47 mmol) and activated MnO₂ (70 g, 800 mmol, Aldrich Cat. No. 217646) in 120 mL CH₂Cl₂ was stirred at the room temperature for 24 hours. The mixture was filtered through Celite (Acros Celite 521, Cat. No. 206350010). The filter cake was washed with CH₂Cl₂ until no UV-active material was seen. The combined CH₂Cl₂ solution was concentrated and passed through a thin silica gel plug, using 25% EtOAc/Hexanes. The filtrate was concentrated to give an off white solid (6.37 g, 71.6% yield).

HPLC Rt: 5.53 min.

¹H-NMR (CDCl₃): δ 10.32 (s, 1H), 8.88 (s, 1H), 2.65 (s, 3H).

Set forth below are methods for preparing some intermediates and examples of the compounds of this invention. Also provided below are methods for testing and using the compounds of this invention for inhibiting mitotic kinases and for treating disorders associated with these kinases. These examples are for illustration purposes only and not intended to limit the scope of this invention in any way.

### Example 1

### 6-(Methylthio)-1H-pyrazolo[3,4-d]pyrimidine

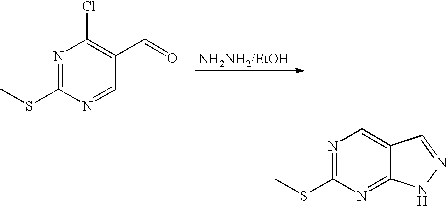

A solution of 4-chloro-2-(methylthio) pyrimidine-5-carbaldehyde (6.3 g, 33 mmol) in 160 mL of EtOH and DIEA (6.6 g, 52 mmol) was cooled on an ice-bath before adding hydrazine (1.8 g, 56 mmol, Alfa Aesar P/N 32728) during 5 minutes. The reaction was left stirring at 0° C. for an additional one hour before placing on an oil bath at 50° C. for another hour. Solvents were removed and the residue washed with water then dried under high vacuum to give a crude product (5.05 g, 91% yield). The compound could be recrystallized from mixture of a methanol and water.

HPLC Rt: 3.91 min.

¹H-NMR (CDCl₃): δ 10.95 (br, s, 1H), 9.00 (s, 1H), 8.10 (s, 1H), 2.66 (s, 3H).

### Example 2

### 1-(1,2-Dihydroacenaphthylen-1-yl)-6-(methylthio)-1H-pyrazolo[3,4-d]pyrimidine

The title compound was synthesized by Mitsunobu coupling between 1,2-dihydroacenaphthylen-1-ol and 6-(methylthio)-1H-pyrazolo[3,4-d]pyrimidine according to General Procedure 1-1.

HPLC Rt: 7.47 min.

¹H-NMR (CDCl₃): δ 8.93 (s, 1H), 7.99 (s, 1H), 7.77 (m, 2H), 7.47 (m, 1H), 7.40 (m, 1H), 7.17 (d, 1H), 6.95 (m, 1H), 4.99 (m, 1H), 4.11 (m, 1H), 3.92 (m, 1H), 2.3 (s, 3H).

### Example 3

### 1-(1,2-Dihydroacenaphthylen-1-yl)-6-(methylsulfonyl)-1H-pyrazolo[3,4-d]pyrimidine

The title compound was synthesized by oxidation of 1-(1,2-dihydroacenaphthylen-1-yl)-6-(methylthio)-1H-pyrazolo[3,4-d]pyrimidine according to General Procedure 1-10.

HPLC Rt: 6.36 min.

¹H-NMR (DMSO-d₆): δ 9.35 (s, 1H), 8.30 (s, 1H), 7.78 (d, 1H), 7.75 (d, 1H), 7.61 (t, 1H), 7.44 (m, 2H), 7.09 (m, 2H), 4.18 (m, 1H), 3.86 (m, 1H), 3.24 (s, 3H).

### Example 4

### 6-(Methylthio)-1-(2-nitrobenzyl)-1H-pyrazolo[3,4-d]pyrimidine

The title compound was synthesized by Mitsunobu coupling between 2-nitro-benzylalcohol and 6-(methylthio)-1H-pyrazolo[3,4-d]pyrimidine according to General Procedure 1.1.

HPLC Rt: 6.79 min.

LC/MS: 302 (M+1).

### Example 5

### 6-(Methylsulfonyl)-1-(2-nitrobenzyl)-1H-pyrazolo[3,4-d]pyrimidine

The title compound was synthesized by oxidation of 6-(methylthio)-1-(2-nitrobenzyl)-1H-pyrazolo[3,4-d]pyrimidine according to General Procedure 1-10.

HPLC Rt: 5.79 min.

LC/MS: 334 (M+1).

### Example 6

### 6-(Methylthio)-1-(4-nitrobenzyl)-1H-pyrazolo[3,4-d]pyrimidine

The title compound was synthesized by Mitsunobu coupling between 4-nitro-benzylalcohol and 6-(methylthio)-1H-pyrazolo[3,4-d]pyrimidine according to General Procedure 1-1.

HPLC Rt: 6.81 min.

LC/MS: 302 (M+1).

### Example 7

### 6-(Methylsulfonyl)-1-(4-nitrobenzyl)-1H-pyrazolo[3,4-d]pyrimidine

The title compound was synthesized by oxidation of 6-(methylsulfonyl)-1-(4-nitrobenzyl)-1H-pyrazolo[3,4-d]pyrimidine according to General Procedure 1-10.

HPLC Rt: 5.854 min.

LC/MS: 334 (M+1).

### Example 8

### (S)-6-(Methylthio)-1-(1-phenylethyl)-1H-pyrazolo[3,4-d]pyrimidine

The title compound was synthesized by Mitsunobu coupling between (S)-1-phenylethanol and 6-(methylthio)-1H-pyrazolo[3,4-d]pyrimidine according to General Procedure 1-1.

HPLC Rt: 6.98 min.

¹H-NMR (CDCl₃): δ 8.89 (s, 1H), 8.02 (s, 1H), 7.36 (m, 5H), 6.18 (q, 1H), 2.64 (s, 3H), 2.01 (d, 3H).

### Example 9

### (S)-6-(Methylsulfonyl)-1-(1-phenylethyl)-1H-pyrazolo[3,4-d]pyrimidine

The title compound was synthesized by oxidation of (S)-6-(methylthio)-1-(1-phenylethyl)-1H-pyrazolo[3,4-d]pyrimidine according to General Procedure 1-10.

HPLC Rt: 5.79 min.

¹H-NMR (CDCl₃) δ 9.30 (s, 1H), 8.34 (s, 1H), 7.35 (m, 5H), 6.35 (q, 1H), 3.43 (s, 3H), 2.06 (d, 3H).

### Example 10

### 1-(6-(Methylthio)-1H-pyrazolo[3,4-d]pyrimidin-1-yl)-2,3-dihydro-1H-inden-4-yl benzoate

The title compound was synthesized by Mitsunobu coupling between 1-hydroxy-2,3-dihydro-1H-inden-4-yl benzoate and 6-(methylthio)-1H-pyrazolo[3,4-d]pyrimidine according to General Procedure 1-1.

HPLC Rt: 8.21 min.

LC/MS is 403(M+1).

### Example 11

### 1-(6-(Methylsulfonyl)-1H-pyrazolo[3,4-d]pyrimidin-1-yl)-2,3-dihydro-1H-inden-4-yl benzoate

The title compound was synthesized by oxidation of 1-(6-(methylthio)-1H-pyrazolo[3,4-d]pyrimidin-1-yl)-2,3-dihydro-1H-inden-4-yl benzoate according to General Procedure 1-10.

HPLC Rt: 7.20 min.

LC/MS: 435(M+1).

### Example 12

### N-(1-(6-(methylthio)-1H-pyrazolo[3,4-d]pyrimidin-1-yl)-2,3-dihydro-1H-inden-4-yl)acetamide

The title compound was synthesized by Mitsunobu coupling between N-(1-hydroxy-2,3-dihydro-1H-inden-4-yl)acetamide and 6-(methylthio)-1H-pyrazolo[3,4-d]pyrimidine according to General Procedure 1-1.

HPLC Rt: 6.10 min.

LC/MS: 340(M+1).

### Example 13

### N-(1-(6-(methylsulfonyl)-1H-pyrazolo[3,4-d]pyrimidin-1-yl)-2,3-dihydro-1H-inden-4-yl)acetamide

The title compound was synthesized by oxidation of N-(1-(6-(methylthio)-1H-pyrazolo[3,4-d]pyrimidin-1-yl)-2,3-dihydro-1H-inden-4-yl)acetamide according to General Procedure 1-10.

HPLC Rt: 5.22 min.

LC/MS: 372(M+1).

### Example 14

### 1-(4-methoxybenzyl)-6-(methylthio)-1H-pyrazolo[3,4-d]pyrimidine

The title compound was synthesized by Mitsunobu coupling between 4-methoxy-benzylalcohol and 6-(methylthio)-1H-pyrazolo[3,4-d]pyrimidine according to General Procedure 1-1.

HPLC Rt: 6.55 min.

¹H-NMR (CDCl₃): δ 8.90 (s, 1H), 7.99 (s, 1H), 7.32 (m, 2H), 6.84 (d, 2H), 5.53 (s, 2H), 3.77 (s, 3H), 2.66 (s, 3H).

### Example 15

### 1-(4-Methoxybenzyl)-6-(methylsulfonyl)-1H-pyrazolo[3,4-d]pyrimidine

The title compound was synthesized by oxidation of 1-(4-methoxybenzyl)-6-(methylsulfonyl)-1H-pyrazolo[3,4-d]pyrimidine according to General Procedure 1-10.

HPLC Rt: 5.54 min.

¹H-NMR (CDCl₃) δ 9.32 (s, 1H), 8.32 (s, 1H), 7.38 (m, 2H), 6.88 (d, 2H), 5.70 (s, 2H), 3.80 (s, 3H), 3.48 (s, 3H).

### Example 16

### (S)-1-(2,3-dihydro-1H-inden-1-yl)-6-(methylthio)-1H-pyrazolo[3,4-d]pyrimidine

The title compound was synthesized by Mitsunobu coupling between (R)-2,3-dihydro-1H-inden-1-ol and 6-(methylthio)-1H-pyrazolo[3,4-d]pyrimidine according to General Procedure 1-1.

HPLC Rt: 7.15 min.

¹H-NMR (CDCl₃): δ 8.95 (s, 1H), 8.01 (s, 1H), 7.36 (d, 1H), 7.28 (q, 1H), 7.16 (t, 1H), 7.01 (d, 1H), 6.55 (t, 1H), 3.39 (m, 1H), 3.10 (m, 1H), 2.76 (m, 1H), 2.66 (m, 1H), 2.63 (s, 3H).

### Example 17

### (S)-1-(2,3-dihydro-1H-inden-1-yl)-6-(methylsulfonyl)-1H-pyrazolo[3,4-d]pyrimidine

The title compound was synthesized by oxidation of (S)-1-(2,3-dihydro-1H-inden-1-yl)-6-(methylthio)-1H-pyrazolo[3,4-d]pyrimidine according to General Procedure 1-10.

HPLC Rt: 5.99 min.

¹H-NMR (CDCl₃): δ 9.34 (s, 1H), 8.31 (s, 1H), 7.39 (d, 1H), 7.31 (q, 1H), 7.17 (t, 1H), 7.00 (d, 1H), 6.69 (t, 1H), 3.45 (s, 1H), 3.43 (m, 3H), 3.13 (m, 1H), 2.80 (m, 1H), 2.66 (m, 1H).

### Example 18

### 6-(Methylthio)-1-(3-nitrobenzyl)-1H-pyrazolo[3,4-d]pyrimidine

The title compound was synthesized by Mitsunobu coupling between 3-nitro-benzylalcohol and 6-(methylthio)-1H-pyrazolo[3,4-d]pyrimidine according to General Procedure 1.1.

HPLC Rt: 6.63 min.

¹H-NMR (CDCl₃): δ 8.93 (s, 1H), 8.27 (m, 1H), 8.16 (m, 1H), 8.05 (s, 1H), 7.69 (d, 1H), 7.52 (t, 1H), 5.70 (s, 2H), 2.65 (s, 3H).

### Example 19

### 6-(Methylsulfonyl)-1-(3-nitrobenzyl)-1H-pyrazolo[3,4-d]pyrimidine

The title compound was synthesized by oxidation of 6-(methylthio)-1-(3-nitrobenzyl)-1H-pyrazolo[3,4-d]pyrimidine according to General Procedure 1-10.

HPLC Rt: 5.83 min.

LC/MS: 334(M+1).

### Example 20

### 1-(5-Methoxy-2,3-dihydro-1H-inden-1-yl)-6-(methylthio)-1H-pyrazolo[3,4-d]pyrimidine

The title compound was synthesized by Mitsunobu coupling between 5-methoxy-2,3-dihydro-1H-inden-1-ol and 6-(methylthio)-1H-pyrazolo[3,4-d]pyrimidine according to General Procedure 1-1.

HPLC Rt: 7.41 min.

LC/MS: 313(M+1).

### Example 21

### 1-(5-Methoxy-2,3-dihydro-1H-inden-1-yl)-6-(methylsulfonyl)-1H-pyrazolo[3,4-d]pyrimidine

The title compound was synthesized by oxidation of 1-(5-methoxy-2,3-dihydro-1H-inden-1-yl)-6-(methylthio)-1H-pyrazolo[3,4-d]pyrimidine according to General Procedure 1-10.

HPLC Rt: 6.32 min.

LC/MS: 345.0 (M+1).

### Example 22

### Ethyl 5-amino-1-(3-methoxyphenyl)-1H-pyrazole-4-carboxylate

The title compound was obtained by condensation of (3-methoxyphenyl)hydrazine with (Z)-ethyl 2-cyano-3-ethoxyacrylate according to General Procedure 1-4.

HPLC Rt: 6.10 min.

¹H-NMR (DMSO-D₆): δ 7.71 (s 1H), 7.45 (t, 1H), 7.15 m, 2H), 6.95 (m 1H), 6.35 (s, 2H), 4.22 (q, 2H), 3.82 (s, 3H), 1.28 (t, 3H).

### Example 23

### Ethyl 5-amino-1-(4-methoxyphenyl)-1H-pyrazole-4-carboxylate

The title compound was obtained by condensation of (4-methoxyphenyl)hydrazine with (Z)-ethyl 2-cyano-3-ethoxyacrylate according to the general procedure 1-4.

HPLC Rt: 5.96 min.

¹H-NMR (DMSO-D₆): δ 7.77 (s 1H), 7.45 (d, 2H), 7.03d, 2H), 5.20 (s, 2H), 4.22 (q, 2H), 3.82 (s, 3H), 1.28 (t, 3H).

### Example 24

### Ethyl 5-amino-1-(naphthalen-1-yl)-1H-pyrazole-4-carboxylate

The title compound was obtained by condensation of naphthalen-1-ylhydrazine with (Z)-ethyl 2-cyano-3-ethoxyacrylate according to General Procedure 1-4.

HPLC Rt: 6.53 min.

¹H-NMR (DMSO-D₆): δ 8.03 (d, 2H), 7.86 (s, 1H), 7.53 (d, 2H), 7.37 (d, 2H), 6.71 (s, 1H), 4.22 (q, 2H), 1.28 (t, 3H).

### Example 25

### Ethyl 5-(3-benzoylthioureido)-1-(3-methoxyphenyl)-1H-pyrazole-4-carboxylate

The title compound was obtained from ethyl 5-amino-1-(3-methoxyphenyl)-1H-pyrazole-4-carboxylate according to General Procedure 1-5.

HPLC Rt: 7.14 min.

LC/MS: 425.0 (M+1).

### Example 26

### Ethyl 5-(3-benzoylthioureido)-1-(4-methoxyphenyl)-1H-pyrazole-4-carboxylate

The title compound was obtained from ethyl 5-amino-1-(4-methoxyphenyl)-1H-pyrazole-4-carboxylate according to the general procedure 1-5.

HPLC Rt: 7.02 min.

¹H-NMR (CDCl₃): δ 12.15 (s, 3H), 1.35 (t, 3H).

### Example 27

### Ethyl 5-(3-benzoylthioureido)-1-(naphthalen-1-yl)-1H-pyrazole-4-carboxylate

The title compound was obtained from ethyl 5-amino-1-(naphthalene-1-yl)-1H-pyrazole-4-carboxylate according to General Procedure 1-5.

HPLC Rt: 7.51 min.

¹H-NMR (DMSO): δ 12.08 (s, 1H), 11.86 (s, 1H), 8.28 (s, 1H), 7.89 (m, 3H), 7.64 (m, 3H), 7.40 (m, 6H), 4.24 (q, 2H), 1.28 (t, 3H).

### Example 28

### 6-Mercapto-1-(3-methoxyphenyl)-1H-pyrazolo[3,4-d]pyrimidin-4-ol

The title compound was obtained by cyclelization of ethyl 5-(3-benzoylthioureido)-1-(3-methoxyphenyl)-1H-pyrazole-4-carboxylate in basic condition according to General Procedure 1-6.

HPLC Rt: 4.96 min.

LC/MS: 275.0 (M+1).

### Example 29

### 6-Mercapto-1-(4-methoxyphenyl)-1H-pyrazolo[3,4-d]pyrimidin-4-ol

The title compound was obtained by cyclelization of ethyl 5-(3-benzoylthioureido)-1-(4-methoxyphenyl)-1H-pyrazole-4-carboxylate in basic condition according to General Procedure 1-6.

HPLC Rt: 4.89 min.

¹H-NMR (DMSO): δ 10.25 (s, 1H), 8.11 (d, 2H), 7.82 (s, 1H), 7.06d, 2H), 3.80 (s, 3H).

### Example 30

### 6-Mercapto-1-(naphthalen-1-yl)-1H-pyrazolo[3,4-d]pyrimidin-4-ol

The title compound was obtained by cyclelization of ethyl 5-(3-benzoylthioureido)-1-(naphthalen-1-yl)-1H-pyrazole-4-carboxylate in basic condition according to General Procedure 1-6.

HPLC Rt: 5.59 min.

¹H-NMR (DMSO-D₆): δ 10.21 (s, 1H), 8.09 (t, 2H), 8.05 (s, 1H), 7.96 (t, 1H), 7.63 (m, 2H), 7.50 (m, 1H), 7.39d, 1H).

### Example 31

### 1-(3-Methoxyphenyl)-6-(methylthio)-1H-pyrazolo[3,4-d]pyrimidin-4-ol

The title compound was obtained from 6-mercapto-1-(3-methoxyphenyl)-1H-pyrazolo[3,4-d]pyrimidin-4-ol according to General Procedure 1-7.

HPLC Rt: 6.14 min.

¹H-NMR (DMSO-D₆): δ 8.23 (s 1H), 7.95 (s, 2H), 7.87s, 1H), 7.79 (d, 1H), 7.46 (t, 1), 6.95 (d, 1H), 3.82 (s, 3H), 2.56 (s, 3H).

### Example 32

### 1-(4-Methoxyphenyl)-6-(methylthio)-1H-pyrazolo[3,4-d]pyrimidin-4-ol

The title compound was obtained from 6-mercapto-1-(4-methoxyphenyl)-1H-pyrazolo[3,4-d]pyrimidin-4-ol according to General Procedure 1-7.

HPLC Rt: 6.00 min.

¹H-NMR (DMSO-D₆): δ 8.10 (d, 2H), 7.72 (s, 1H), 7.03d, 2H), 3.78 (s, 3H), 2.39 (s, 3H).

### Example 33

### 1-(Naphthalen-1-yl)-6-(methylthio)-1H-pyrazolo[3,4-d]pyrimidin-4-ol

The title compound was obtained from 6-mercapto-1-(naphthalen-1-yl)-1H-pyrazolo[3,4-d]pyrimidin-4-ol according to General Procedure 1-7.

HPLC Rt: 6.39 min.

¹H-NMR (DMSO-D₆): δ 12.65 (s 1H), 8.34 (s, 1H), 8.13 (dd, 2H), 7.67 (m, 2H), 7.57 (m, 1H), 7.30m, 2H), 2.23 (s, 3H).

### Example 34

### 4-Chloro-1-(3-methoxyphenyl)-6-(methylthio)-1H-pyrazolo[3,4-d]pyrimidine

The title compound was obtained from 1-(3-methoxyphenyl)-6-(methylthio)-1H-pyrazolo[3,4-d]pyrimidin-4-ol with POCl₃ according to General Procedure 1-8.

HPLC Rt: 8.26 min.

¹H-NMR (DMSO-D₆): δ 8.61 (s 1H), 7.85 (s, 1H), 7.76d, 1H), 7.50 (t, 1H), 6.98 (d, 1H), 3.85 (s, 3H), 2.67 (s, 3H).

### Example 35

### 4-Chloro-1-(4-methoxyphenyl)-6-(methylthio)-1H-pyrazolo[3,4-d]pyrimidine

The title compound was obtained from 1-(4-methoxyphenyl)-6-(methylthio)-1H-pyrazolo[3,4-d]pyrimidin-4-ol with POCl₃ according to General Procedure 1-8.

HPLC Rt: 8.06 min.

¹H-NMR (DMSO): δ 8.59 (s 1H), 8.02 (d, 2H), 7.18 (d, 2H), 3.82 (s, 3H), 2.67 (s, 3H).

### Example 36

### 4-Chloro-1-(naphthalen-1-yl)-6-(methylthio)-1H-pyrazolo[3,4-d]pyrimidine

The title compound was obtained from 1-(naphthalen-1-yl)-6-(methylthio)-1H-pyrazolo[3,4-d]pyrimidin-4-ol with POCl₃ according to the general procedure 1-8.

HPLC Rt: 8.27 min.

¹H-NMR (DMSO): S 8.73 (s 1H), 8.21 (d, 1H), 8.13 (d, 1H), 7.78 (m, 3H), 7.55 (m, 2H), 2.39 (s, 3H).

### Example 37

### N-(4-(1H-pyrazolo[3,4-d]pyrimidin-6-ylamino)phenyl)-N-(methylsulfonyl)methanesulfonamide

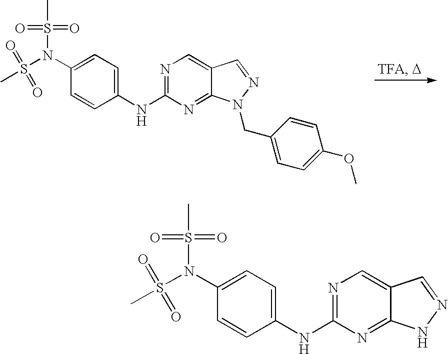

Synthesis of the title compound was conducted according to procedure described by R. N. Misra in Bioorg. Med. Chem. Lett. EN, 2006, 13 (6), 1133-1136. Specifically, N-(4-(1-(4-methoxybenzyl)-1H-pyrazolo[3,4-d]pyrimidin-6-ylamino)phenyl)-N-(methylsulfonyl)methanesulfonamide (0.34 g, 0.68 mmol) was dissolved in 5 ml TFA and the solution heated under reflux for 16 hours. TFA was then removed under reduced pressure to give a greenish oily residue which converted to a solid product upon adding ice-cold water. The solid was filtered, washed with a generous amount of DCM before it was washed again with ether to give 0.248 g (yield 96%) of the title product.

Provided below in Tables 1-4 are additional specific examples of the compounds of Formula (I) which were prepared according to the procedures described above and tested by the methods described hereinafter.

TABLE 1
COMPOUNDS CONTAINING NAPHTHALENYL OR TETRAHYDRONAPHTALENYL GROUP
AS R₂ IN FORMULA (I)

| | | HPLC | | |
|---|---|---|---|---|
| Example | | RT | | |
| No. | Compound Name | (mins.) | LC/MS | ¹H-NMR (CDCl₃ unless noted) |
| 38 | N-(methylsulfonyl)-N-(4-(1- | 6.95 | 509 (M + 1) | |
| | (naphthalen-1-yl)-1H-pyrazolo[3,4- | | | |
| | d]pyrimidin-6- | | | |
| | ylamino)phenyl)methanesulfonamide | | | |
| 39 | N-methyl-N-(4-(1-(naphthalen-1- | 6.89 | 443 (M − 1) | |
| | yl)-1H-pyrazolo[3,4-d]pyrimidin-6- | | | |
| | ylamino)phenyl)methanesulfonamide | | | |
| 40 | N-(4-(1-(naphthalen-1-yl)-1H- | 6.71 | 457 (M + 1) | |
| | pyrazolo[3,4-d]pyrimidin-6- | | | |
| | ylamino)phenyl)cyclopropanesulfonamide | | | |
| 41 | 1-(methylsulfonyl)-N-(4-(1- | 6.53 | 509 (M + 1) | |
| | (naphthalen-1-yl)-1H-pyrazolo[3,4- | | | |
| | d]pyrimidin-6- | | | |
| | ylamino)phenyl)methanesulfonamide | | | |
| 42 | N-(4-(1-(naphthalen-1-yl)-1H- | 6.47 | | 2.94 (s, 3H), 6.20 (s, 1H), 7.04 (d, |
| | pyrazolo[3,4-d]pyrimidin-6- | | | J = 8.67 Hz, 2H), 7.28 (s, 1H), 7.53 (m, |
| | ylamino)phenyl)methanesulfonamide | | | 5H), 7.70 (t, J = 8.66 Hz, 2H), 8.18 (t, |
| | | | | J = 8.66 Hz, 2H), 8.28 (s, 1H), 9.01 (s, |
| | | | | 1H). |
| 43 | N¹-(1-(naphthalen-2-yl)-1H- | 6.84 | 353 (M + 1) | |
| | pyrazolo[3,4-d]pyrimidin-6- | | | |
| | yl)benzene-1,4-diamine | | | |
| 44 | ethyl 2-(4-(1-(naphthalen-1-yl)-1H- | 6.89 | | (DMSO-d6) 1.30 (t, J = 5.68 Hz, 3H), |
| | pyrazolo[3,4-d]pyrimidin-6- | | | 4.31 (dd, 2H), 7.47-7.67 (m, 9H), |
| | ylamino)phenylamino)-2- | | | 8.15 (t, J = 8.52 Hz, 2H), 8.46 (s, 1H), 9.16 (s, |
| | oxoacetate | | | 1H), 9.95 (s, 1H), 10.60 (s, 1H). |
| 45 | N-(4-(1-(naphthalen-2-yl)-1H- | 7.09 | 431 (M + 1) | |
| | pyrazolo[3,4-d]pyrimidin-6- | | | |
| | ylamino)phenyl)methanesulfonamide | | | |
| 46 | 3,3,3-trifluoro-N-(4-(1-(naphthalen- | 7.16 | 535 (M + 23) | |
| | 1-yl)-1H-pyrazolo[3,4-d]pyrimidin- | | | |
| | 6-ylamino)phenyl)propane-1- | | | |
| | sulfonamide | | | |
| 47 | methyl 4-(1-(naphthalen-1-yl)-1H- | 6.69 | | 3.78 (s, 3H), 6.50 (s, 1H), 7.19 (t, |
| | pyrazolo[3,4-d]pyrimidin-6- | | | J = 8.67 Hz, 2H), 7.36 (s, 1H), |
| | ylamino)phenylcarbamate | | | 7.48-7.59 (m, 5H), 7.64 (d, J = 8.67 Hz, 1H), |
| | | | | 7.66 (d, J = 8.67 Hz, 1H), 8.01 (t, |
| | | | | J = 8.67 Hz, 2H), 8.25 (s, 1H), 8.98 (s, 1H). |
| 48 | N-(3,4-dimethoxyphenyl)-1- | 6.92 | 398 (M + 1) | |
| | (naphthalen-1-yl)-1H-pyrazolo[3,4- | | | |
| | d]pyrimidin-6-amine | | | |
| 49 | N-(4-(4-methylpiperazin-1- | 6.07 | 436 (M + 1) | |
| | yl)phenyl)-1-(naphthalen-2-yl)-1H- | | | |
| | pyrazolo[3,4-d]pyrimidin-6-amine | | | |
| 50 | ethyl 2-hydroxy-4-(1-(naphthalen- | 6.72 | 439 (M − 1) | |
| | 1-yl)-1H-pyrazolo[3,4-d]pyrimidin- | | | |
| | 6-ylamino)phenylcarbamate | | | |
| 51 | N-(4-(1H-1,2,4-triazol-1- | 7.80 | 403 (M − 1) | |
| | yl)phenyl)-1-(naphthalen-1-yl)-1H- | | | |
| | pyrazolo[3,4-d]pyrimidin-6-amine | | | |
| 52 | methyl 3-(4-(1-(naphthalen-1-yl)- | 6.41 | 451 (M − 1) | |
| | 1H-pyrazolo[3,4-d]pyrimidin-6- | | | |
| | ylamino)phenylamino)-3- | | | |
| | oxopropanoate | | | |
| 53 | N¹-(1-(naphthalen-1-yl)-1H- | 5.36 | | 6.52 (d, J = 8.52 Hz, 2H), 7.28 (d, |
| | pyrazolo[3,4-d]pyrimidin-6- | | | J = 8.52 Hz, 2H), 7.31 (t, J = 8.55 Hz, 1H), |
| | yl)benzene-1,4-diamine | | | 7.50-7.64 (m, 3H), 7.79 (d, J = 8.64 Hz, |
| | | | | 1H), 7.81 (d, J = 8.64 Hz, 1H), 8.01 (t, |
| | | | | J = 8.66 Hz, 2H), 8.21 (s, 1H), 8.93 (s, |
| | | | | 1H). |
| 54 | N-(4-(4-methylpiperazin-1- | 5.64 | 434.9 (M + 1) | 2.40 (t, J = 5.64 Hz, 4H), 2.64 (s, 3H), |
| | yl)phenyl)-1-(naphthalen-1-yl)-1H- | | | 3.16 (t, J = 5.64 Hz, 4H), 6.77 (d, |
| | pyrazolo[3,4-d]pyrimidin-6-amine | | | J = 8.56 Hz, 2H), 7.02 (s, 1H), |
| | | | | 7.28-7.64 (m, 5H), 7.78 (d, J = 8.65 Hz, 1H), |
| | | | | 7.98 (d, J = 8.65 Hz, 1H), 8.03 (t, J = 8.55 Hz, |
| | | | | 2H), 8.23 (s, 1H), 8.96 (s, 1H). |
| 55 | N-(4-fluoro-3-methoxyphenyl)-1- | 7.52 | 384 (M − 1) | |
| | (naphthalen-1-yl)-1H-pyrazolo[3,4- | | | |
| | d]pyrimidin-6-amine | | | |
| 56 | N-(4-ethoxyphenyl)-1-(naphthalen- | 7.52 | | 1.30 (t, J = 5.42 Hz, 3H), 4.06 (dd, |
| | 1-yl)-1H-pyrazolo[3,4-d]pyrimidin- | | | J = 5.42 Hz, 2H), 6.75 (d, J = 8.46 Hz, 2H), |
| | 6-amine | | | 7.19 (s, 1H), 7.41-7.81 (m, 7H), 8.03 (t, |
| | | | | J = 8.64 Hz, 2H), 8.24 (s, 1H), 8.97 (s, |
| | | | | 1H). |
| 57 | 3-(1-(naphthalen-1-yl)-1H- | 6.55 | | 4.07 (s, 1H), 6.44 (d, J = 8.46 Hz, 1H), |
| | pyrazolo[3,4-d]pyrimidin-6- | | | 6.75 (d, J = 8.46 Hz, 1H), 7.06 (t, |
| | ylamino)phenol | | | J = 8.52 Hz, 1H), 7.34 (d, J = 8.52 Hz, 2H), |
| | | | | 7.57~7.76 (m, 4H), 7.85 (d, J = 8.52 Hz, |
| | | | | 1H), 8.01 (t, J = 8.52 Hz, 2H), 8.28 (s, |
| | | | | 1H), 9.01 (s, 1H). |
| 58 | 2-(4-(1-(naphthalen-1-yl)-1H- | 6.42 | 382 (M − 1) | |
| | pyrazolo[3,4-d]pyrimidin-6- | | | |
| | ylamino)phenyl)ethanol | | | |
| 59 | 2-methyl-5-(1-(naphthalen-1-yl)- | 6.84 | 366 (M − 1) | |
| | 1H-pyrazolo[3,4-d]pyrimidin-6- | | | |
| | ylamino)phenol | | | |
| 60 | 2-methoxy-5-(1-(naphthalen-1-yl)- | 6.46 | 384.9 (M + 1) | 3.85 (s, 3H), 5.50 (s, 1H), 6.70 (d, |
| | 1H-pyrazolo[3,4-d]pyrimidin-6- | | | J = 8.45 Hz, 1H), 7.00 (d, J = 8.45 Hz, 1H), |
| | ylamino)phenol | | | 7.20 (s, 2H), 7.45~7.80 (m, 5H), 8.00 (t, |
| | | | | J = 8.75 Hz, 2H), 8.25 (s, 1H), 8.92 (s, |
| | | | | 1H). |
| 61 | N-(4-methoxyphenyl)-1- | 7.19 | | 3.77 (s, 3H), 6.80 (d, J = 8.76 Hz, 2H), |
| | (naphthalen-1-yl)-1H-pyrazolo[3,4- | | | 7.20 (s, 1H), 7.50 (d, J = 8.76 Hz, 2H), |
| | d]pyrimidin-6-amine | | | 7.60~7.80 (m, 5H), 8.01 (t, J = 8.24 Hz, |
| | | | | 2H), 8.24 (s, 1H), 8.96 (s, 1H). |
| 62 | N-(3-chloro-4-methoxyphenyl)-1- | 7.62 | 402 (M + 1) | |
| | (naphthalen-1-yl)-1H-pyrazolo[3,4- | | | |
| | d]pyrimidin-6-amine | | | |
| 63 | N-(4-(1-(naphthalen-1-yl)-1H- | 6.20 | | (D6-DMSO) 2.19 (s, 3H), 7.27 (d, |
| | pyrazolo[3,4-d]pyrimidin-6- | | | J = 8.46 Hz, 2H), 7.50~7.77 (m, 7H), |
| | ylamino)phenyl)acetamide | | | 8.16 (t, J = 8.55 Hz, 2H), 8.43 (s, 1H), 9.14 (s, |
| | | | | 1H), 9.73 (s, 1H), 9.84 (s, 1H). |
| 64 | 4-(1-(naphthalen-1-yl)-1H- | 6.23 | 354 (M + 1) | |
| | pyrazolo[3,4-d]pyrimidin-6- | | | |
| | ylamino)phenol | | | |
| 65 | 2-methoxy-5-(1-(naphthalen-2-yl)- | 7.21 | 384 (M + 1) | |
| | 1H-pyrazolo[3,4-d]pyrimidin-6- | | | |
| | ylamino)phenol | | | |
| 66 | N-(4-methoxy-3- | 7.78 | 436 (M + 1) | |
| | (trifluoromethyl)phenyl)-1- | | | |
| | (naphthalen-1-yl)-1H-pyrazolo[3,4- | | | |
| | d]pyrimidin-6-amine | | | |
| 67 | N-(4-(1-(4-(benzyloxy)-5,6,7,8- | 8.151 | 619 (M + 1) | |
| | tetrahydronaphthalen-1-yl)-1H- | | | |
| | pyrazolo[3,4-d]pyrimidin-6- | | | |
| | ylamino)phenyl)-N- | | | |
| | (methylsulfonyl)methanesulfonamide | | | |
| 68 | 1-(4-(benzyloxy)-5,6,7,8- | 6.882 | 560 (M + 1) | |
| | tetrahydronaphthalen-1-yl)-N-(4-(4- | | | |
| | methylpiperazin-1-yl)phenyl)-1H- | | | |
| | pyrazolo[3,4-d]pyrimidin-6-amine | | | |
| 69 | N1-(1-(4-(benzyloxy)-5,6,7,8- | 6.699 | 463 (M + 1) | |
| | tetrahydronaphthalen-1-yl)-1H- | | | |
| | pyrazolo[3,4-d]pyrimidin-6- | | | |
| | yl)benzene-1,4-diamine | | | |
| 70 | 4-(6-(4-aminophenylamino)-1H- | 5.145 | 373 (M + 1) | |
| | pyrazolo[3,4-d]pyrimidin-1-yl)- | | | |
| | 5,6,7,8-tetrahydronaphthalen-1-ol | | | |
| 71 | tert-butyl 4-(1-(4-(benzyloxy)- | 8.537 | 555 (M + 1) | |
| | 5,6,7,8-tetrahydronaphthalen-1-yl)- | | | |
| | 1H-pyrazolo[3,4-d]pyrimidin-6- | | | |
| | ylamino)piperidine-1-carboxylate | | | |
| 72 | 4-(6-(4-(N- | 6.967 | 629 (M + 23) | |
| | (methylsulfonyl)methylsulfonamido)phenylamino)- | | | |
| | 1H-pyrazolo[3,4- | | | |
| | d]pyrimidin-1-yl)-5,6,7,8- | | | |
| | tetrahydronaphthalen-1-yl | | | |
| | methanesulfonate | | | |
| 73 | 4-(6-(4- | 6.572 | 529 (M + 1) | |
| | (methylsulfonamido)phenylamino)- | | | |
| | 1H-pyrazolo[3,4-d]pyrimidin-1-yl)- | | | |
| | 5,6,7,8-tetrahydronaphthalen-1-yl | | | |
| | methanesulfonate | | | |
| 74 | N-(4-(1-(4-(benzyloxy)-5,6,7,8- | 7.780 | 619 (M + 1) | |
| | tetrahydronaphthalen-1-yl)-1H- | | | |
| | pyrazolo[3,4-d]pyrimidin-6- | | | |
| | ylamino)phenyl)methanesulfonamide | | | |
| 75 | N-(4-(1-(4-hydroxy-5,6,7,8- | 6.572 | 551 (M + 23) | |
| | tetrahydronaphthalen-1-yl)-1H- | | | |
| | pyrazolo[3,4-d]pyrimidin-6- | | | |
| | ylamino)phenyl)-N- | | | |
| | (methylsulfonyl)methanesulfonamide | | | |
| 76 | N-(4-(1-(4-hydroxy-5,6,7,8- | 6.052 | 451 (M + 1) | |
| | tetrahydronaphthalen-1-yl)-1H- | | | |
| | pyrazolo[3,4-d]pyrimidin-6- | | | |
| | ylamino)phenyl)methanesulfonamide | | | |
| 77 | 4-(6-(4-(4-methylpiperazin-1- | 5.262 | 456 (M + 1) | |
| | yl)phenylamino)-1H-pyrazolo[3,4- | | | |
| | d]pyrimidin-1-yl)-5,6,7,8- | | | |
| | tetrahydronaphthalen-1-ol | | | |
| 78 | 1-(4-(benzyloxy)-5,6,7,8- | 7.515 | 533 (M + 1) | |
| | tetrahydronaphthalen-1-yl)-N-(4- | | | |
| | morpholinophenyl)-1H- | | | |
| | pyrazolo[3,4-d]pyrimidin-6-amine | | | |
| 79 | 4-(6-(4-morpholinophenylamino)- | 5.660 | 443 (M + 1) | |
| | 1H-pyrazolo[3,4-d]pyrimidin-1-yl)- | | | |
| | 5,6,7,8-tetrahydronaphthalen-1-ol | | | |
| 80 | N-(4-morpholinophenyl)-1- | 5.96 | | 8.80 (s, 1H), 7.90 (s, 1H), 7.52 (d, 2H), |
| | (1,2,3,4-tetrahydronaphthalen-1- | | | 7.39 (s, 1H), 7.15 (m, 2H), 7.00 (t, 1H), |
| | yl)-1H-pyrazolo[3,4-d]pyrimidin-6- | | | 6.91 (d, 2H), 6.69 (d, 1H), 6.09 (m, 1H), |
| | amine | | | 3.89 (t, 3H), 3.13 (t, 4H), 3.01 (m, 1H), |
| | | | | 2.92 (m, 1H), 2.48 (m, 1H), 2.17 (m, |
| | | | | 2H), 1.96 (m, 1H). |

TABLE 2
COMPOUNDS CONTAINING DIHYDROINDENYL GROUP AS R₂ IN FORMULA (I)

| | | HPLC | | |
|---|---|---|---|---|
| | | data | | |
| Example | | (Rt: | | |
| No. | Compound Name | Mins.) | LC/MS | ¹H-NMR (CDCl₃) |
| 81 | (S)-1-(2,3-dihydro-1H-inden-1-yl)- | 5.24 | 426 (M + 1) | |
| | N-(4-(4-methylpiperazin-1- | | | |
| | yl)phenyl)-1H-pyrazolo[3,4- | | | |
| | d]pyrimidin-6-amine | | | |
| 82 | (S)-4-(1-(2,3-dihydro-1H-inden-1- | 6.2 | | 12.63 (s, 1H), 10.33 (s, 1H), 9.07 (s, |
| | yl)-1H-pyrazolo[3,4-d]pyrimidin-6- | | | 1H), 7.84 (m, 2H), 7.67 (d, 1H), 7.26 (t, |
| | ylamino)benzoic acid | | | 1H), 7.15 (t, 1H), 6.94 (d, 1H), 6.44 (t, |
| | | | | 1H), 3.27 (m, 1H), 3.07 (m, 1H), |
| | | | | 2.65 (m, 1H), 2.55 (m, 1H). |
| 83 | (S)—N-(4-(1-(2,3-dihydro-1H-inden- | 6.61 | 499.8 (M + 1) | |
| | 1-yl)-1H-pyrazolo[3,4-d]pyrimidin- | | | |
| | 6-ylamino)phenyl)-N- | | | |
| | (methylsulfonyl)methanesulfonamide | | | |
| 84 | (S)—N¹-(1-(2,3-dihydro-1H-inden-1- | 5.11 | 343.0 (M + 1) | 8.80 (s, 1H), 7.89 (s, 1H), 7.42 (d, 2H), |
| | yl)-1H-pyrazolo[3,4-d]pyrimidin-6- | | | 7.35 (d, 1H), 7.30 (m, 1H), 7.15 (t, 1H), |
| | yl)benzene-1,4-diamine | | | 7.03 (d, 1H) 6.67 (d, 2H), 6.44 (t, 1H), |
| | | | | 3.61 (b, 2H), 3.37 (m, 1H), 3.07 (m, 1H), |
| | | | | 2.65 (m, 2H). |
| 85 | (S)-1-(2,3-dihydro-1H-inden-1-yl)- | 5.79 | | 8.79 (s, 1H), 7.88 (s, 1H), 7.56 (m, 2H), |
| | N-(4-morpholinophenyl)-1H- | | | 7.36 (s, 1H), 7.29 (m, 2H), 7.14 (t, 1H), |
| | pyrazolo[3,4-d]pyrimidin-6-amine | | | 7.02 (d, 1H), 6.92 (d, 2H), 6.41 (t, 1H), |
| | | | | 3.88 (t, 4H), 3.35 (m, 1H), 3.14 (t, 4H), |
| | | | | 3.05 (m, 1H), 2.68 (m, 2H). |
| 86 | (S)-1-(2,3-dihydro-1H-inden-1-yl)- | 6.72 | | 8.82 (s, 1H), 7.92 (s, 1H), 7.58 (m, 2H), |
| | N-(4-(4-(propylsulfonyl)piperazin- | | | 7.39 (m, 2H), 7.29 (m, 1H), 7.14 (t, 1H), |
| | 1-yl)phenyl)-1H-pyrazolo[3,4- | | | 7.02 (d, 1H), 6.97 (d, 2H), 6.41 (t, 1H), |
| | d]pyrimidin-6-amine | | | 3.47 (t, 4H), 3.35 (m, 1H), 3.22 (t, 4H), |
| | | | | 3.05 (m, 1H), 2.97 (m, 2H), 2.71 (m, |
| | | | | 2H), 1.95 (m, 2H), 1.1 (t, 3H). |
| 87 | 4-(1-(2,3-dihydro-1H-inden-1-yl)- | 6.21 | | 10.33 (s, 1H), 9.07 (s, 1H), 8.12 (s, 1H), |
| | 1H-pyrazolo[3,4-d]pyrimidin-6- | | | 7.87 (m, 4H), 7.42 (d, 1H), 7.29 (t, 1H), |
| | ylamino)benzoic acid | | | 7.12 (t, 1H), 6.92 (d, 1H), 6.41 (t, 1H), |
| | | | | 3.25 (m, 1H), 3.05 (m, 1H), 2.68 (m, |
| | | | | 1H), 2.56 (m, 1H). |
| 88 | 1-(2,3-dihydro-1H-inden-1-yl)-N- | 5.57 | | 8.90 (s, 1H), 7.92 (s, 1H), 7.60 (t, 1H), |
| | (3-(piperazin-1-yl)phenyl)-1H- | | | 7.42 (s, 1H), 7.35 (d, 1H), 7.30 (m, 1H), |
| | pyrazolo[3,4-d]pyrimidin-6-amine | | | 7.22 (m, 1H), 7.15 (m, 2H), 7.03 (d, 1H) |
| | | | | 6.67 (dd, 1H), 6.44 (t, 1H), (3.37 (m, |
| | | | | 1H), 3.19 (t, 4H), 3.07 (m, 1H), 2.974 (t, |
| | | | | 4H), 2.65 (m, 2H). |
| 89 | N¹-(1-(2,3-dihydro-1H-inden-1-yl)- | 5.06 | | 8.80 (s, 1H), 7.89 (s, 1H), 7.42 (d, 2H), |
| | 1H-pyrazolo[3,4-d]pyrimidin-6- | | | 7.35 (d, 1H), 7.30 (m, 1H), 7.15 (t, 1H), |
| | yl)benzene-1,4-diamine | | | 7.03 (d, 1H) 6.67 (d, 2H), 6.44 (t, 1H), |
| | | | | 3.61 (b, 2H), 3.37 (m, 1H), 3.07 (m, 1H), |
| | | | | 2.65 (m, 2H). |
| 90 | N-(4-(1-(2,3-dihydro-1H-inden-1- | 6.13 | | 8.83 (s, 1H), 7.93 (s, 1H), 7.67 (d, 2H), |
| | yl)-1H-pyrazolo[3,4-d]pyrimidin-6- | | | 7.56 (s, 1H), 7.35 (d, 1H), 7.30 (m, 1H), |
| | ylamino)phenyl)methanesulfonamide | | | 7.23 (d, 2H), 7.15 (t, 1H), 7.01 (d, 1H), |
| | | | | 6.66 (s, 1H), 6.44 (t, 1H), 3.37 (m, 1H), |
| | | | | 3.07 (m, 1H), 3.00 (s, 3H), 2.65 (m, 2H). |
| 91 | 1-(2,3-dihydro-1H-inden-1-yl)-N- | 6.74 | | 8.81 (s, 1H), 7.90 (s, 1H), 7.57 (m, 2H), |
| | (4-(4-(propylsulfonyl)piperazin-1- | | | 7.54 (s, 1H), 7.44 (d, 1H), 7.29 (t, 1H), |
| | yl)phenyl)-1H-pyrazolo[3,4- | | | 7.16 (t, 1H), 7.05 (d, 1H), 6.96 (m, 2H), |
| | d]pyrimidin-6-amine | | | 6.43 (t, 1H), 3.44 (m, 4H), 3.39 (m, 1H), |
| | | | | 3.28 (m, 4H), 3.28 (t, 4H), 3.11 (m, 1H), |
| | | | | 2.97 (m, 2H), 2.72 (m, 2H), 1.93 (m, |
| | | | | 2H), 1.12 (t, 3H), |
| 92 | (S)-1-(2,3-dihydro-1H-inden-1-yl)- | 6.24 | | 8.81 (s, 1H), 7.90 (s, 1H), 7.57 (m, 2H), |
| | N-(4-(4-(methylsulfonyl)piperazin- | | | 7.54 (s, 1H), 7.38 (d, 1H), 7.29 (t, 1H), |
| | 1-yl)phenyl)-1H-pyrazolo[3,4- | | | 7.16 (t, 1H), 7.05 (d, 1H), 6.96 (m, 2H), |
| | d]pyrimidin-6-amine | | | 6.43 (t, 1H), 3.44 (m, 4H), 3.39 (m, 1H), |
| | | | | 3.28 (m, 4H), 3.28 (t, 4H), 3.11 (m, 1H), |
| | | | | 2.85 (s, 3H), 2.72 (m, 2H). |
| 93 | (S)-1-(2,3-dihydro-1H-inden-1-yl)- | 5.31 | 411.9 (M + 1) | |
| | N-(4-(piperazin-1-yl)phenyl)-1H- | | | |
| | pyrazolo[3,4-d]pyrimidin-6-amine | | | |
| 94 | N-(4-(1-(2,3-dihydro-1H-inden-1- | 7.74 | | 11.65 (s, 1H), 8.73 (s, 1H), 8.14 (s, 1H), |
| | yl)-1H-pyrazolo[3,4-d]pyrimidin-6- | | | 7.89 (s, 2H), 7.87 (s, 1H), 7.65 (m, 4H), |
| | ylamino)phenyl)benzamide | | | 7.52 (m, 1H), 7.50 (m, 2H), 7.34 (t, 1H), |
| | | | | 7.18 (t, 1H), 7.02 (d, 1H), 6.38 (t, 1H), |
| | | | | 3.36 (m, 1H), 3.13 (m, 1H), 2.78 (m, |
| | | | | 1H). |
| 95 | (S)-5-(1-(2,3-dihydro-1H-inden-1- | 6.14 | 374 (M + 1) | |
| | yl)-1H-pyrazolo[3,4-d]pyrimidin-6- | | | |
| | ylamino)-2-methoxyphenol | | | |
| 96 | 1-(2,3-dihydro-1H-inden-1-yl)-N- | 5.22 | 411.9 (M + 1) | 8.81 (s, 1H), 7.90 (s, 1H), 7.57 (m, 2H), |
| | (4-(piperazin-1-yl)phenyl)-1H- | | | 7.54 (s, 1H), 7.38 (d, 1H), 7.29 (t, 1H), |
| | pyrazolo[3,4-d]pyrimidin-6-amine | | | 7.16 (t, 1H), 7.05 (d, 1H), 6.96 (m, 2H), |
| | | | | 6.43 (t, 1H), 3.32 (m, 1H), 3.13 (m, 4H). |
| 97 | 1-(2,3-dihydro-1H-inden-1-yl)-N- | 5.79 | | 8.79 (s, 1H), 7.88 (s, 1H), 7.56 (m, 2H), |
| | (4-morpholinophenyl)-1H- | | | 7.36 (s, 1H), 7.29 (m, 2H), 7.14 (t, 1H), |
| | pyrazolo[3,4-d]pyrimidin-6-amine | | | 7.02 (d, 1H), 6.92 (d, 2H), 6.41 (t, 1H), |
| | | | | 3.88 (t, 4H), 3.35 (m, 1H), 3.14 (t, 4H), |
| | | | | 3.05 (m, 1H), 2.68 (m, 2H). |
| 98 | 3-(4-(4-(1-(2,3-dihydro-1H-inden- | 5.34 | | 8.79 (s, 1H), 7.88 (s, 1H), 7.56 (m, 2H), |
| | 1-yl)-1H-pyrazolo[3,4-d]pyrimidin- | | | 7.36 (m, 2H), 7.26 (m, 1H), 7.14 (t, 1H), |
| | 6-ylamino)phenyl)piperazin-1- | | | 7.02 (d, 1H), 6.92 (d, 2H), 6.41 (t, |
| | yl)propan-1-ol | | | 1H), 5.10 (b, 1H), 3.84 (t, 2H), 3.35 (m, |
| | | | | 1H), 3.14 (t, 4H), 3.05 (m, 2H), 2.72 (m, |
| | | | | 8H), 1.78 (m, 2H). |
| 99 | 4-(1-(2,3-dihydro-1H-inden-l-yl)- | 5.12 | 384.4 (M − 1) | |
| | 1H-pyrazolo[3,4-d]pyrimidin-6- | | | |
| | ylamino)benzohydrazide | | | |
| 100 | 2-(4-(4-(1-(2,3-dihydro-1H-inden- | 5.6 | 497.9 (M + 1) | |
| | 1-yl)-1H-pyrazolo[3,4-d]pyrimidin- | | | |
| | 6-ylamino)phenyl)piperazin-1- | | | |
| | yl)ethyl acetate | | | |
| 101 | 1-(2,3-dihydro-1H-inden-1-yl)-N- | 6.07 | | 8.79 (s, 1H), 8.20 (s, 1H), 7.88 (s, 1H), |
| | (4-(4-((4-methoxy-3,5- | | | 7.56 (d, 2H), 7.35 (d, 1H), 7.27 (m, 2H), |
| | dimethylpyridin-2- | | | 7.13 (t, 1H), 7.02 (d, 1H), 6.89 (d, 2H), |
| | yl)methyl)piperazin-1-yl)phenyl)- | | | 6.38 (t, 1H), 3.77 (s, 3H), 3.66 (s, 2H), |
| | 1H-pyrazolo[3,4-d]pyrimidin-6- | | | 3.33 (m, 1H), 2.70 (t, 4H), 3.20 (s, 3H), |
| | amine | | | 2.25 (s, 3H). |
| 102 | 3-(4-(4-(1-(2,3-dihydro-1H-inden- | 5.68 | | 8.79 (s, 1H), 7.88 (s, 1H), 7.56 (m, 2H), |
| | 1-yl)-1H-pyrazolo[3,4-d]pyrimidin- | | | 7.43 (s, 1H), 7.36 (d, 1H), 7.29 (m, 1H), |
| | 6-ylamino)phenyl)piperazin-1- | | | 7.14 (t, 1H), 7.02 (d, 1H), 6.92 (d, 2H), |
| | yl)propyl acetate | | | 6.41 (t, 1H), 4.16 (m, 3H), 3.35 (m, 1H), |
| | | | | 3.17 (t, 4H), 3.05 (m, 1H), 2.62 (m, 6H), |
| | | | | 2.48 (t, 2H), 2.05 (s, 3H), 1.83 (t, 2H). |
| 103 | 2-(4-(4-(1-(2,3-dihydro-1H-inden- | 5.27 | | 8.79 (s, 1H), 7.88 (s, 1H), 7.56 (m, 2H), |
| | 1-yl)-1H-pyrazolo[3,4-d]pyrimidin- | | | 7.36 (m, 1H), 7.29 (m, 2H), 7.14 (t, 1H), |
| | 6-ylamino)phenyl)piperazin-1- | | | 7.02 (d, 1H), 6.92 (d, 2H), 6.41 (t, 1H), |
| | yl)ethanol | | | 3.77 (t, 2H), 3.35 (m, 1H), 3.14 (t, 4H), |
| | | | | 3.05 (m, 1H), 2.71 (m, 6H), 2.61 (t, 2H). |
| 104 | 1-((S)-2,3-dihydro-1H-inden-1-yl)- | 7.46 | 608.2 (M + 1) | |
| | N-(3-(1-((R)-2,3-dihydro-1H- | | | |
| | inden-1-yl)-1H-pyrazolo[3,4- | | | |
| | d]pyrimidin-6-yloxy)-4- | | | |
| | methoxyphenyl)-1H-pyrazolo[3,4- | | | |
| | d]pyrimidin-6-amine | | | |
| 105 | (S)-ethyl 4-(1-(2,3-dihydro-1H- | 7.6 | | 8.89 (s, 1H), 8.22 (d, 2H), 7.97 (s, 1H), |
| | inden-1-yl)-1H-pyrazolo[3,4- | | | 7.67 (d, 2H), 7.56 (s, 1H), 7.45 (d, 1H), |
| | d]pyrimidin-6-ylamino)benzoate | | | 7.35 (m, 1H), 7.15 (t, 1H), 7.01 (d, 1H), |
| | | | | 6.44 (t, 1H), 4.41 (m, 2H), 3.52 (m, 1H), |
| | | | | 3.37 (m, 1H), 3.07 (m, 1H), 3.00 (s, 3H), |
| | | | | 2.65 (m, 2H), 1.38 (t, 3H). |
| 106 | (S)-1-(2,3-dihydro-1H-inden-1-yl)- | 5.79 | | 8.79 (s, 1H), 7.88 (s, 1H), 7.56 (m, 2H), |
| | N-(4-morpholinophenyl)-1H- | | | 7.36 (s, 1H), 7.29 (m, 2H), 7.14 (t, 1H), |
| | pyrazolo[3,4-d]pyrimidin-6-amine | | | 7.02 (d, 1H), 6.92 (d, 2H), 6.41 (t, 1H), |
| | | | | 3.88 (t, 4H), 3.35 (m, 1H), 3.14 (t, 4H), |
| | | | | 3.05 (m, 1H), 2.68 (m, 2H). |
| 107 | 1-(2,3-dihydro-1H-inden-2-yl)-N- | 5.94 | | 8.79 (s, 1H), 7.89 (s, 1H), 7.29 (m, 5H), |
| | (4-morpholinophenyl)-1H- | | | 6.89 (d, 2H), 5.71 (m, 1H), 3.88 (t, 4H), |
| | pyrazolo[3,4-d]pyrimidin-6-amine | | | 3.66 (m, 2H), 3.48 (m, 2H), 3.11 (m, |
| | | | | 1H). |
| 108 | N-(4-(1-(4-hydroxy-2,3-dihydro-1H- | 6.159 | 515 (M + 1) | |
| | inden-1-yl)-1H-pyrazolo[3,4- | | | |
| | d]pyrimidin-6-ylamino)phenyl)-N- | | | |
| | (methylsulfonyl)methanesulfonamide | | | |
| 109 | (S)—N-(4-(1-(2,3-dihydro-1H-inden- | 7.301 | 527 (M + 1) | |
| | 1-yl)-1H-pyrazolo[3,4-d]pyrimidin- | | | |
| | 6-ylamino)phenyl)-N- | | | |
| | (ethylsulfonyl)ethanesulfonamide | | | |
| 110 | 1-(6-(4-(4-methylpiperazin-1- | 4.896 | 442 (M + 1) | |
| | yl)phenylamino)-1H-pyrazolo[3,4- | | | |
| | d]pyrimidin-1-yl)-2,3-dihydro-1H- | | | |
| | inden-4-ol | | | |
| 111 | 1-(6-(4-(N- | 7.507 | 619 (M + 1) | |
| | (methylsulfonyl)methylsulfonamido)phenylamino)- | | | |
| | 1H-pyrazolo[3,4- | | | |
| | d]pyrimidin-1-yl)-2,3-dihydro-1H- | | | |
| | inden-4-yl benzoate | | | |
| 112 | 1-(6-(4-(N- | 4.830 | 595 (M + 1) | |
| | (methylsulfonyl)methylsulfonamido)phenylamino)- | | | |
| | 1H-pyrazolo[3,4- | | | |
| | d]pyrimidin-1-yl)-2,3-dihydro-1H- | | | |
| | inden-4-yl dihydrogen phosphate | | | |
| 113 | (S)-1-chloro-N- | 7.228 | | 8.89 (s, 1H); 7.98 (s, 1H), 7.76 (d, |
| | (chloromethylsulfonyl)-N-(4-(1- | | | 2H), 7.55 (s, 1H), 7.44 (d, 2H), |
| | (2,3-dihydro-1H-inden-1-yl)-1H- | | | 7.39 (d, 1H), 7.31 (t, 1H), 7.17 (t, 1H), |
| | pyrazolo[3,4-d]pyrimidin-6- | | | 7.03 (d, 1H), 5.06 (s, 4H), 3.36 (m, 1H), |
| | ylamino)phenyl)methanesulfonamide | | | 3.13 (m, 1H), 2.76 (m, 2H) |
| 114 | N-(4-(1-(6,7-dihydro-5H- | 6.771 | 543 (M + 1) | |
| | indeno[5,6-d][1,3]dioxol-5-yl)-1H- | | | |
| | pyrazolo[3,4-d]pyrimidin-6- | | | |
| | ylamino)phenyl)-N- | | | |
| | (methylsulfonyl)methanesulfonamide | | | |
| 115 | N-(1-(6-(4-(N- | 6.023 | 556 (M + 1) | |
| | (methylsulfonyl)methylsulfonamido)phenylamino)- | | | |
| | 1H-pyrazolo[3,4- | | | |
| | d]pyrimidin-1-yl)-2,3-dihydro-1H- | | | |
| | inden-5-yl)acetamide | | | |
| 116 | N-(4-(1-(4-hydroxy-2,3-dihydro-1H- | 5.762 | 437 (M + 1) | |
| | inden-1-yl)-1H-pyrazolo[3,4- | | | |
| | d]pyrimidin-6- | | | |
| | ylamino)phenyl)methanesulfonamide | | | |
| 117 | 1-(6-(4-(N- | 6.543 | 619 (M + 1) | |
| | (methylsulfonyl)methylsulfonamido)phenylamino)- | | | |
| | 1H-pyrazolo[3,4- | | | |
| | d]pyrimidin-1-yl)-2,3-dihydro-1H- | | | |
| | inden-5-yl benzoate | | | |
| 118 | (S,Z)-4-(1-(2,3-dihydro-1H-inden-1- | 6.402 | 386 (M + 1) | |
| | yl)-1H-pyrazolo[3,4-d]pyrimidin-6- | | | |
| | ylamino)-N′-hydroxybenzimidamide | | | |
| 119 | 1-(6-(4-(4-methylpiperazin-1- | 6.415 | 546 (M + 1) | |
| | yl)phenylamino)-1H-pyrazolo[3,4- | | | |
| | d]pyrimidin-1-yl)-2,3-dihydro-1H- | | | |
| | inden-4-yl benzoate | | | |
| 120 | 1-(6-(4-aminophenylamino)-1H- | 4.729 | 359 (M + 1) | |
| | pyrazolo[3,4-d]pyrimidin-1-yl)-2,3- | | | |
| | dihydro-1H-inden-4-ol | | | |
| 121 | 1-(6-(4-aminophenylamino)-1H- | 6.286 | 463 (M + 1) | |
| | pyrazolo[3,4-d]pyrimidin-1-yl)-2,3- | | | |
| | dihydro-1H-inden-4-yl benzoate | | | |
| 122 | di-tert-butyl 1-(6-(4-(N- | 7.446 | 707 (M + 1) | |
| | (methylsulfonyl)methylsulfonamido)phenylamino)- | | | |
| | 1H-pyrazolo[3,4- | | | |
| | d]pyrimidin-1-yl)-2,3-dihydro-1H- | | | |
| | inden-4-yl phosphate | | | |
| 123 | (S)-4-(1-(2,3-dihydro-1H-inden-1- | 6.567 | 449 (M + 1) | |
| | yl)-1H-pyrazolo[3,4-d]pyrimidin-6- | | | |
| | ylamino)-N- | | | |
| | (methylsulfonyl)benzamide | | | |
| 124 | N-(4-(1-(5-((2- | 6.637 | | 8.87 (s, 1H); 7.97 (s, 1H), 7.81 (d, |
| | methoxyethoxy)methoxy)-2,3- | | | 2H), 7.52 (s, 1H), 7.34 (d, 2H), |
| | dihydro-1H-inden-1-yl)-1H- | | | 7.09 (s, 1H), 6.97 (d, 1H), 6.87 (dd, |
| | pyrazolo[3,4-d]pyrimidin-6- | | | 1H), 6.42 (t, 1H), 5.28 (s, 2H), 3.84 (m, |
| | ylamino)phenyl)-N- | | | 2H), 3.58 (m, 2H), 3.44 (s, 6H), |
| | (methylsulfonyl)methanesulfonamide | | | 3.39 (s, 3H), 3.32 (m, 1H), 3.10 (m, |
| | | | | 1H), 2.76 (m, 1H), 2.72 (m, 1H) |
| 125 | N-(1-(6-(4-aminophenylamino)-1H- | 4.703 | 400 (M + 1) | |
| | pyrazolo[3,4-d]pyrimidin-1-yl)-2,3- | | | |
| | dihydro-1H-inden-5-yl)acetamide | | | |
| 126 | (S)-4-(1-(2,3-dihydro-1H-inden-1- | 7.04 | | 8.00 (s, 1H); 7.70 (s, 1H), 7.68 (s, |
| | yl)-1H-pyrazolo[3,4-d]pyrimidin-6- | | | 1H), 8.60 (m, 3H), 7.44 (d, 1H), |
| | ylamino)benzonitrile | | | 7.34 (t, 1H), 7.19 (t, 1H), 7.04 (d, 1H), |
| | | | | 6.47 (t, 1H), 3.36 (m, 1H), 3.13 (m, |
| | | | | 1H), 2.76 (m, 2H) |
| 127 | (S)—N-(4-(1H-tetrazol-5-yl)phenyl)- | 6.39 | 396 (M + 1) | |
| | 1-(2,3-dihydro-1H-inden-1-yl)-1H- | | | |
| | pyrazolo[3,4-d]pyrimidin-6-amine | | | |
| 128 | N1-(1-(5-methoxy-2,3-dihydro-1H- | 5.090 | 373 (M + 1) | |
| | inden-1-yl)-1H-pyrazolo[3,4- | | | |
| | d]pyrimidin-6-yl)benzene-1,4- | | | |
| | diamine | | | |
| 129 | N-(4-(1-(5-methoxy-2,3-dihydro- | 6.856 | 529 (M + 1) | |
| | 1H-inden-1-yl)-1H-pyrazolo[3,4- | | | |
| | d]pyrimidin-6-ylamino)phenyl)-N- | | | |
| | (methylsulfonyl)methanesulfonamide | | | |
| 130 | (S)-1-(2,3-dihydro-1H-inden-1-yl)- | 6.939 | 401 (M + 1) | |
| | N-(4-nitrophenethyl)-1H- | | | |
| | pyrazolo[3,4-d]pyrimidin-6-amine | | | |
| 131 | (S)—N-(4-aminophenethyl)-1-(2,3- | 5.251 | 371 (M + 1) | |
| | dihydro-1H-inden-1-yl)-1H- | | | |
| | pyrazolo[3,4-d]pyrimidin-6-amine | | | |
| 132 | (S)—N-(4-(2-(1-(2,3-dihydro-1H- | 6.583 | 527 (M + 1) | |
| | inden-1-yl)-1H-pyrazolo[3,4- | | | |
| | d]pyrimidin-6- | | | |
| | ylamino)ethyl)phenyl)-N- | | | |
| | (methylsulfonyl)methanesulfonamide | | | |

TABLE 3
COMPOUNDS CONTAINING DIHYDROACETANAPHTHYLENYL GROUP
AS R₂ IN FORMULA (I)

| | | HPLC | | |
|---|---|---|---|---|
| | | data | | |
| | | (Rt: | | |
| 133 | Compound Name | Minutes.) | LC/MS | ¹H-NMR (CDCl₃) |
| 134 | 1-(1,2-dihydroacenaphthylen-1- | 5.6 | | 8.80 (s, 1H), 7.89 (s, 1H), 7.76 (t, 2H), |
| | yl)-N-(4-(4-methylpiperazin-1- | | | 7.56 (t, 1H), 7.45 (t, 1H), 7.40 (s, 1H), |
| | yl)phenyl)-1H-pyrazolo[3,4- | | | 7.38 (m, 3H), 7.20 (d, 1H), 6.89 (m, |
| | d]pyrimidin-6-amine | | | 1H), 6.75 (d, 2H), 4.02 (m, 2H), 3.18 (t, |
| | | | | 4H), 2.60 (m, 4H), 2.38 (s, 3H). |
| 135 | N-(4-(1-(1,2- | 6.84 | | 8.89 (s, 1H), 7.89 (s, 1H), 7.76 (t, 2H), |
| | dihydroacenaphthylen-1-yl)-1H- | | | 7.56 (t, 1H), 7.45 (t, 1H), 7.40 (s, 1H), |
| | pyrazolo[3,4-d]pyrimidin-6- | | | 7.38 (m, 3H), 7.20 (d, 1H), 7.03 (d, 2H), |
| | ylamino)phenyl)-N- | | | 6.89 (m, 1H), 4.02 (m, 2H), 3.41 (s, 6H). |
| | (methylsulfonyl)methanesulfonamide | | | |
| 136 | N¹-(1-(1,2-dihydroacenaphthylen- | 5.31 | 379 (M + 1) | |
| | 1-yl)-1H-pyrazolo[3,4- | | | |
| | d]pyrimidin-6-yl)benzene-1,4- | | | |
| | diamine | | | |
| 137 | 1-(1,2-dihydroacenaphthylen-1- | 6.09 | | 8.80 (s, 1H), 7.89 (s, 1H), 7.76 (t, 2H), |
| | yl)-N-(4-morpholinophenyl)-1H- | | | 7.56 (t, 1H), 7.45 (t, 1H), 7.40 (s, 1H), |
| | pyrazolo[3,4-d]pyrimidin-6-amine | | | 7.38 (m, 3H), 7.20 (d, 1H), 6.89 (m, |
| | | | | 1H), 6.75 (d, 2H), 4.02 (m, 2H), 3.87 (t, |
| | | | | 4H), 3.09 (t, 4H). |
| 138 | 4-(1-(1,2-dihydroacenaphthylen- | 7.3 | | 10.27 (s, 1H), 9.08 (s, 1H), 8.12 (s, 1H), |
| | 1-yl)-1H-pyrazolo[3,4- | | | 7.79 (m, 6H), 7.65 (t, 1H), 7.49 (d, 2H), |
| | d]pyrimidin-6- | | | 7.18 (d, 1H), 6.95 (m, 1H), 4.15 (m, 2H). |
| | ylamino)benzonitrile | | | |
| 139 | 1-(1,2-dihydroacenaphthylen-1- | 6.4 | | 8.91 (s, 1H), 8.02 (s, 1H), 7.83 (m, 2H), |
| | yl)-N-(4-(2-(trimethylstannyl)- | | | 7.65 (t, 1H), 7.51 (s, 1H), 7.40 (t, 1H), |
| | 2H-tetrazol-5-yl)phenyl)-1H- | | | 7.35 (m, 3H), 7.20 (d, 1H), 6.89 (m, |
| | pyrazolo[3,4-d]pyrimidin-6-amine | | | 1H), 4.15 (m, 2H), 3.88 (1H). |
| 140 | 1-(1,2-dihydroacenaphthylen-1- | 5.42 | 447.9 (M + 1) | 8.78 (s, 1H), 7.88 (s, 1H), 7.63 (d, 2H), |
| | yl)-N-(4-(piperazin-1-yl)phenyl)- | | | 7.34 (s, 1H), 7.00 (d, 2H), 4.85 (m, 1H), |
| | 1H-pyrazolo[3,4-d]pyrimidin-6- | | | 3.23 (t, 4H), 2.64 (t, 4H), 2.38 (s, 3H), |
| | amine | | | 2.21 (m, 2H), 1.90 (m, 2H), 1.74 (m, |
| | | | | 6H). |

TABLE 4
COMPOUNDS CONTAINING INDOLYL GROUPS AS R2 IN FORMULA (I)

| | | HPLC | | |
|---|---|---|---|---|
| Example | | (Rt, | LC/ | |
| No. | Compound Name | Min) | MS | ¹H-NMR (CDCl₃ unless noted) |
| 141 | N-(4-(1-(1H-indol-4-yl)-1H- | 6.28 | 498 (M + 1) | |
| | pyrazolo[3,4-d]pyrimidin-6- | | | |
| | ylamino)phenyl)-N- | | | |
| | (methylsulfonyl)methanesulfonamide | | | |
| 142 | N-(4-(1-(1H-indol-6-yl)-1H- | 6.61 | 498 (M + 1) | |
| | pyrazolo[3,4-d]pyrimidin-6- | | | |
| | ylamino)phenyl)-N- | | | |
| | (methylsulfonyl)methanesulfonamide | | | |
| 143 | N-(4-(1-(1H-indol-5-yl)-1H- | 6.38 | 498 (M + 1) | |
| | pyrazolo[3,4-d]pyrimidin-6- | | | |
| | ylamino)phenyl)-N- | | | |
| | (methylsulfonyl)methanesulfonamide | | | |
| 144 | 1-(1H-indol-4-yl)-N-(4-(4- | 5.14 | 425 (M + 1) | |
| | methylpiperazin-1-yl)phenyl)-1H- | | | |
| | pyrazolo[3,4-d]pyrimidin-6-amine | | | |
| 145 | N-(4-(1-(1-methyl-1H-indol-5-yl)- | 6.80 | 512 (M + 1) | |
| | 1H-pyrazolo[3,4-d]pyrimidin-6- | | | |
| | ylamino)phenyl)-N- | | | |
| | (methylsulfonyl)methanesulfonamide | | | |
| 146 | 1-(7-methyl-1H-indol-4-yl)-N-(4- | 5.23 | 439 (M + 1) | |
| | (4-methylpiperazin-1-yl)phenyl)- | | | |
| | 1H-pyrazolo[3,4-d]pyrimidin-6- | | | |
| | amine | | | |
| 147 | tert-butyl 7-methyl-4-(6-(4-(4- | 6.71 | | 8.88 (s, 1H), 8.13 (s, 1H), 7.69 (d, 1H), |
| | methylpiperazin-1- | | | 7.55 (m, 3H), 7.34 (s, 1H), 7.24 (d, 1H), |
| | yl)phenylamino)-1H- | | | 6.87 (d, 2H), 6.82 (d, 1H), 3.17 (m, 4H), |
| | pyrazolo[3,4-d]pyrimidin-1-yl)- | | | 2.70 (s, 3H), 2.61 (m, 4H), 2.37 (s, 3H), |
| | 1H-indole-1-carboxylate | | | 1.65 (s, 9H). |
| 148 | tert-butyl 4-(6-(4-(4- | 6.60 | | 8.89 (s, 1H), 8.23 (d, 1H), 8.14 (s, 1H), |
| | methylpiperazin-1- | | | 7.88 (d, 1H), 7.63 (d, 1H), 7.57 (d, 2H), |
| | yl)phenylamino)-1H- | | | 7.46 (t, 1H), 7.32 (s, 1H), 6.945 (d, |
| | pyrazolo[3,4-d]pyrimidin-1-yl)- | | | 1H), 6.88 (d, 2H), 3.18 (m, 4H), |
| | 1H-indole-1-carboxylate | | | 2.63 (m, 4H), 2.38 (s, 3H), 1.69 (s, 9H). |
| 149 | 1-((6-fluoro-4H- | 5.47 | | 8.80 (s, 1H), 7.93 (s, 1H), 7.60 (d, 2H), |
| | benzo[d][1,3]dioxin-8-yl)methyl)- | | | 7.20 (s, 1H), 6.96 (d, 2H), 6.65 (m, 2H), |
| | N-(4-morpholinophenyl)-1H- | | | 5.51 (s, 2H), 5.27 (s, 2H), 4.88 (s, 2H), |
| | pyrazolo[3,4-d]pyrimidin-6-amine | | | 3.89 (t, 3H), 3.15 (t, 4H). |

TABLE 5
COMPOUNDS CONTAINING OTHER GROUP AS R₂ IN FORMULA (I)

| | | HPLC | | |
|---|---|---|---|---|
| | | data | | |
| Example | | (Rt, | | |
| No. | Compound Name | mins.) | LC/MS | ¹H-NMR (CDCl₃) |
| 150 | N-(4-morpholinophenyl)-1- | 5.73 | | 8.80 (s, 1H), 8.44 (m, 1H), 7.92 (s, 1H), |
| | (naphthalen-1-ylmethyl)-1H- | | | 7.90 (m, 1H), 7.84 (s, 1H), 7.67 (d, 2H), |
| | pyrazolo[3,4-d]pyrimidin-6-amine | | | 7.57 (d, 1H), 7.51 (m, 3H), 7.38 (s, 1H), |
| | | | | 6.97 (d, 2H), 5.97 (s, 2H), 3.91 (t, 4H), |
| | | | | 3.18 (t, 4H). |
| 151 | 1-butyl-N-(4-(piperidin-1- | 5.34 | | 8.78 (s, 1H), 7.89 (s, 1H), 7.60 (d, 2H), |
| | yl)phenyl)-1H-pyrazolo[3,4- | | | 7.20 (s, 1H), 7.00 (d, 2H), 4.37 (t, 2H), |
| | d]pyrimidin-6-amine | | | 3.15 (t, 4H), 1.95 (m, 2H), 1.60 (m, 6H), |
| | | | | 1.38 (m, 2H), 0.98 (t, 3H). |
| 152 | ethyl 2-(6-(4-(piperidin-1- | 4.78 | | 8.80 (s, 1H), 7.93 (s, 1H), 7.55 (d, 2H), |
| | yl)phenylamino)-1H- | | | 7.25 (d, 1H), 6.96 (d, 2H), 5.11 (s, 2H), |
| | pyrazolo[3,4-d]pyrimidin-1- | | | 4.27 (m, 2H), 3.15 (t, 4H), 1.76 (t, 4H), |
| | yl)acetate | | | 1.60 (m, 2H), 1.31 (m, 5H) |
| 153 | 1-methyl-N-(4-(piperidin-1- | 4.62 | | 1.30 (t, J = 7.12 Hz, 2H, CH₂), 1.75 (m, |
| | yl)phenyl)-1H-pyrazolo[3,4- | | | 4H, 2CH₂), 3.15 (t, J = 5.30 Hz, 4H, |
| | d]pyrimidin-6-amine | | | 2CH₂), 4.00 (s, 3H, CH₃), 6.99 (d, |
| | | | | J = 8.50 Hz, 2H, CH), 7.20 (s, 1H, |
| | | | | NH7.17 (d, J = 8.50 Hz, 2H, 2CH), |
| | | | | 7.89 (s, 1H, CH), 8.79 (s, 1H, CH). |
| 154 | 1-isopropyl-N-(4-piperidin-1- | 4.94 | | 8.78 (s, 1H), 7.89 (s, 1H), 7.60 (d, 2H), |
| | yl)phenyl)-1H-pyrazolo[3,4- | | | 7.20 (s, 1H), 7.00 (d, 2H), 5.01 (m, 1H), |
| | d]pyrimidin-6-amine | | | 3.15 (t, 4H), 2.04 (s, 6H), 1.78 (m, 4H), |
| | | | | 1.58 (m, 6H). |
| 155 | N-(4-(1-(3-aminopropyl)-1H- | 4.660 | 440 (M + 1) | |
| | pyrazolo[3,4-d]pyrimidin-6- | | | |
| | ylamino)phenyl)-N- | | | |
| | (methylsulfonyl)methanesulfonamide | | | |
| 156 | N-(methylsulfonyl)-N-(4-(1- | 6.301 | 425 (M + 1) | |
| | propyl-1H-pyrazolo[3,4- | | | |
| | d]pyrimidin-6- | | | |
| | ylamino)phenyl)methanesulfonamide | | | |
| 157 | N-(4-(1-butyl-1H-pyrazolo[3,4- | 6.522 | 439 (M + 1) | |
| | d]pyrimidin-6-ylamino)phenyl)- | | | |
| | N- | | | |
| | (methylsulfonyl)methanesulfonamide | | | |
| 158 | N-(4-(1-(4-hydroxybutyl)-1H- | 5.403 | 455 (M + 1) | 8.80 (s, 1H), 7.93 (s, 1H), 7.60 (d, 2H), |
| | pyrazolo[3,4-d]pyrimidin-6- | | | 7.40 (s, 1H), 7.35 (m, 2H), 7.30 (m, |
| | ylamino)phenyl)-N- | | | 2H), 6.96 (d, 2H), 6.09 (q, 1H), 3.95 (t, |
| | (methylsulfonyl)methanesulfonamide | | | 3H), 3.19 (t, 4H), 2.03 (d, 3H). |
| 159 | 4-(6-(4-(N- | 6.120 | 497 (M + 1) | 8.80 (s, 1H), 7.93 (s, 1H), 7.60 (d, 2H), |
| | (methylsulfonyl)methylsulfonamido)phenylamino)- | | | 7.20 (s, 1H), 6.96 (d, 2H), 6.65 (m, 2H), |
| | 1H- | | | 5.51 (s, 2H), 5.27 (s, 2H), 4.88 (s, 2H), |
| | pyrazolo[3,4-d]pyrimidin-1- | | | 3.89 (t, 3H), 3.15 (t, 4H). |
| | yl)butyl acetate | | | |
| 160 | N-(4-(1-ethyl-1H-pyrazolo[3,4- | 5.929 | 411 (M + 1) | |
| | d]pyrimidin-6-ylamino)phenyl)- | | | |
| | N- | | | |
| | (methylsulfonyl)methanesulfonamide | | | |
| 161 | N-(4-(1-isopropyl-1H- | 6.241 | 425 (M + 1) | 8.80 (s, 1H), 7.93 (s, 1H), 7.66 (d, 2H), |
| | pyrazolo[3,4-d]pyrimidin-6- | | | 7.39 (d, 1H), 7.34 (s, 1H), 6.96 (d, 2H), |
| | ylamino)phenyl)-N- | | | 6.43 (d, 1H), 6.36 (m, 1H), 5.51 (s, 2H), |
| | (methylsulfonyl)methanesulfonamide | | | 3.17 (t, 3H), 3.09 (t, 4H). |
| 162 | N-(4-(1-methyl-1H-pyrazolo[3,4- | 5.697 | 397 (M + 1) | |
| | d]pyrimidin-6-ylamino)phenyl)- | | | |
| | N- | | | |
| | (methylsulfonyl)methanesulfonamide | | | |

### Example 127

### Aurora Kinase A (AKA) Kinase Assay

The assay was conducted as described by Toji, S. et al. in Genes to Cells, 9: 383-397 (2004), which is incorporated herein by reference in its entirety.

6His-tagged Lats2 substrate (5 ug/mL in PBS) was coated onto a 96-well HisGrab plate (Catalog No. 15142, Pierce Chemical, Rockford, Ill., USA) previously blocked with TBST containing 5% BSA and 1% milk powder. Aurora kinase A (MBL International, Woburn, Mass., USA, # CY-E1165-1) was diluted (1:200) in a kinase reaction buffer (20 mM HEPES, 1 mM DTT, 50 mM MgCl₂, 50 uM ATP, pH 7.5) and autoactivated at 30° C. for 60 minutes. A compound of this invention was then added and the mixture was incubated at 30° C. for 60 minutes. After the incubation, Aurora kinase A was added to the Lats2 coated HisGrab plate and incubated for one hour at 30° C., then washed twice with PBS containing 0.05% Tween-20 (PBST). Anti-Phospho-Lats2-Ser83 monoclonal antibody (MBL International, #ST-3B11) was diluted (1:500) in antibody dilution buffer (TBST with 5% BSA and 1% milk powder), added to each well, and then incubated at room temperature for 30 minutes. Each well was then washed twice with PBST. Horse radish peroxidase (HRP) conjugated goat anti-mouse IgG (Jackson Immunoresearch, West Grove, Pa., USA, #115-035-003) was diluted (1:4000) in TBST with BSA and milk, added to each well, and then incubated at room temperature for 30 minutes. Each well was washed 5 times with PBST, and then to it was added 100 uL of TMB Ultra HRP substrate (Pierce Chemical, #34028), and the mixture incubated for 5 minutes at room temperature. To each well was then added 100 uL of 1N H₂SO₄, and the absorbance was measured at a wavelength of 450 nM using a spectrophotometric plate reader. Aurora kinase incubated with DMSO as the inhibitor (control) is defined as 100% activity. EC₅₀ is defined as the concentration of compound which gives 50% inhibition of Aurora Kinase A.

Most of the tested compounds exhibited an IC₅₀ value of less than 2 μM, some less than 0.2 μM, some less than 0.08 μM and some even less than 0.01 μM.

The results show that the tested compounds of formula (I) exhibited high inhibitory effect on the Aurora Kinase A.

### Example 78

### Aurora Kinase A (AKA) Kinase Assay

The assay was similar to the assay described in Example 77 above. Specifically, a Z-Lyte Kinase Assay Ser/Thr 1 Peptide Kit (Invitrogen, # PV3174) and a synthetic peptide substrate Ser-Thr 1 peptide labeled with a donor fluorophore (coumarin) (Invitrogen, # PV3196) and an acceptor fluorophore (fluorescein) that made up a FRET pair were used. All dilutions were performed in 1× Reaction Buffer (50 mM HEPES-pH 7.5, 10 mM MgCl₂, 1 mM EGTA, 0.01% Brij-35 from 5× stock (Invitrogen, PV3189).

The primary 10 uL reaction involved Aurora Kinase B (Invitrogen # PV3970), 64 uM ATP(PV3227), 2 uM Ser-Thr 1 Peptide, and a compound of this invention (1% in DMSO). The reaction was incubated for 1 hour at the room temperature.

Also used was a 0% control which consisted of 64 uM ATP, 2 uM Ser-Thr 1 Peptide, without Aurora Kinase B, and a 100% control which consisted of 64 uM ATP, 2 uM Phospho Ser-Thr 1 Peptide (Invitrogen # PV3211), without Aurora Kinase B.

In the secondary reaction, 5 uL of a site-specific protease (the Development Reagent A, Invitrogen, # PV3295) was added at a 1:2048 dilution into Development Buffer (Invotrigen, # P3127) for 1 hour at the room temperature. The protease cleaved non-Phosphorylated peptide disrupting FRET interaction of the two fluorophores, while phosphorylated peptide was uncleaved which maintained the FRET interaction. The reaction was read on an M5 spectrophotometer at Excitation 400 nm, and Emission 445 nm and 520 nm.

The Emission ratio (Em)=coumarin (C) emission signal (at 445 nm)/Fluorescein (F) emission signal (at 520 nm).

Percent Phosphorylation=[1−((Em ratio×F100%)−C100%)/((C0%−C100%)+(Em ratio×(F100%−F0%)))].

The testes compounds also showed effective inhibition of Aurora Kinase B. Most of the tested compounds exhibited an IC₅₀ value of less than 2.0 μM, some less than 0.6 μM, some less than 0.2 μM, and some even less than 0.005 μM.

### Example 128

### CDK1 Kinase Assay

A Z-Lyte Kinase Assay Ser/Thr 18 Peptide Kit (Invitrogen, Carlsbad, Calif., USA, #PV4319) was used for this assay and the assay was performed as per manufacturer's instructions. The assay uses a synthetic peptide substrate (Ser-Thr 18 peptide, Invitrogen # PV4320) that was labeled with a donor fluorophore (coumarin) and an acceptor fluorophore (fluorescein) that make up a FRET pair. All dilutions were performed with a 1× Reaction Buffer made of 50 mM HEPES-pH 7.5, 10 mM MgCl₂, 1 mM EGTA, 0.01% Brij-35.

The primary 10 μl reaction involved 0.62 ug/ml CDK1/Cyclin B (Invitrogen, # PV3292), 34 uM ATP, 2 uM Ser-Thr 18 Peptide. A compound of this invention was dissolved in DMSO to 1%. A 0% control was included with 34 uM ATP, 2 uM Ser-Thr 18 Peptide, no enzyme; and a 100% control contained 34 uM ATP, 2 uM Phospho Ser-Thr 18 Peptide (Invitrogen, # PV4321), no enzyme. The reaction was incubated at the room temperature for 1 hour.

In the secondary reaction, 5 uL of a site-specific protease (the Development Reagent A, Invitrogen, # PV3295) was diluted (1:1024) in Development Buffer (#P3127) at the room temperature over 1 hour. The protease cleaved non-Phosphorylated peptide disrupting FRET interaction of the two fluorophores, while phosphorylated peptide was uncleaved which maintains FRET interaction. The reaction mixture was read on an M5 spectrophotometer at Excitation 400 nm, and Emission 445 nm and 520 nm.

The Emission ratio (Em)=coumarin (C) emission signal (at 445 nm)/Fluorescein (F) emission signal (at 520 nm).

Percent Phosphorylation=[1−((Em ratio×F100%)−C100%)/((C0%−C100%)+(Em ratio×(F100%−F0%)))].

IC₅₀ is defined as the concentration of compound which gives 50% inhibition of CDK1 kinase activity.

The results show that the tested compounds exhibited high inhibitory effect on the CDK1 kinase activity. Most of the tested compounds exhibited an IC₅₀ value of less than 2 μM, some less than 0.4 μM, some less than 0.1 μM, and some even less than 0.01 μM.

### Example 129

### G2M Cell Cycle Arrest Assay

100,000 K562 leukemia cells were incubated with increasing concentrations (0-0.001-0.003-0.01-0.03 uM or 0.1-0.3-1-3-30-100 uM) of a compound of this invention in Dulbecco's Modified Eagle Media containing 10% FBS at 37° C. in 10% CO₂ for 24 hours in 200 uL culture volumes in 96-well plates. The cells were washed once in DPBS and then fixed in ice cold 70% ethanol at 4° C. for 30 minutes. After washing once in DPBS, the cells were resuspended in DPBS containing 0.2% Tween-20 for 30 minutes. The cells were washed once again in DPBS, resupsended in a solution of 25 ug/mL propidium iodide, 0.002% NP-40, and 12.5 ug/mL RNAse A. The cellular DNA content was measured on a FACSCALIBUR flow cytometer equipped with an Argon-ion laser that emits 15 mW of 488 nm light for excitation of the propidium iodide fluorescent DNA intercalating dye. The minimum effective concentration was defined as the concentration of inhibitor at which the percentage of cells in G2M exceeded the percentage of cells in G1.

Results are shown as G2M in the tables and showed that the minimum effective concentrations ranged from 0.03 nM to 100 uM to cause G2M cell cycle arrest in K562 leukemia cells.

Most of the tested compounds exhibited an IC₅₀ value of less than 10 μM, some less than 3 μM, some some even less than 0.3 μM.

### Example 130

### HCT116 growth inhibition assay

HCT116 (colon) carcinoma cells were dispensed into 96-well plates (100 uL per well, 20000 cells per mL) and allowed to adhere overnight using standard cell culture conditions. The cultures were then incubated with a compound of this invention under standard culture conditions for 5 days. 3-(4,5-dimethylthiazol-2-yl)-5-(3-carboxymethoxyphenyl)-2-(4-sulfophenyl)-2H-tetrazolium inner salt (MTS), a novel tetrazolium compound (Promega, Madison, Wis., USA, G1111) was added at a concentration of 2 mg/mL and phenazine methyl sulfate at a concentration of 50 ug/mL were mixed 20:1, and 20 uL of the mixture was added to the cultures and allowed to develop for several hours. The color change was used to measure viability at OD490 nm on a 96-well plate spectrophotometric plate reader.

The IC₅₀ was defined as the concentration of compound which gives a 50% inhibition of growth of the HCT116 tumor cell line.

The tested compounds typically exhibited an IC50 value of less than 6.0 μM, some less than 0.8 μM, some less than 0.2 μM, and even some less than 0.08 μM.

### Example 131

### In Vivo Protocol

Six- to eight-week old Balb/C and nu/nu athymic female mice were obtained from Charles River Laboratories The mice were maintained in ventilated caging in a room with a 12 hour light/dark cycle. Food and water were provided ad libitum. Animals were identified by the use of bar coded chips. Experiments were carried out under Biogen Idec IACUC protocol SD34-07 and the guidelines for the proper and human use of animals in research established by the Institute for Laboratory Animal Research (ILAR).

### Example 132

### Mouse Tumor Study

Tumor fragments (approximately 2 mm³) or 5×10⁶ tumor cells were inoculated subcutaneously in the right or left flank of the animal. Mice with established tumors (50-200 mm³) were selected for study (n=7-10/treatment group). Tumor dimensions were measured using calipers and tumor volumes were calculated using the equation for an ellipsoid sphere (1×w²)/2=mm³, where 1 and w refer to the larger and smaller dimensions collected at each measurement.

The test compounds were formulated and administered orally (p.o.) or via the intraperitoneal cavity (IP) at a dose volume of 10 mL/kg. The vehicle alone was administered to control groups. Animals were dosed five days per week (Monday through Friday) for four to six consecutive weeks. Animals were weighed and the tumors were measured twice per week.

Mice were followed until tumor volumes in the control group reached approximately 1000 mm³ and were sacrificed by CO₂ euthanasia. The mean tumor volumes of each group were calculated. The change in mean treated tumor volume was divided by the change in mean control tumor volume, multiplied by 100 and subtracted from 100% to give the tumor growth inhibition for each group. Statistical analysis was performed using the standard T-test and using GraphPad Prism© Software. The results showed that the tested compound effectively reduced the tumor volume.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-55. (canceled)

56. A compound of Formula (I), a prodrug, a polymorph, a tautomer, an enantiomer, a stereoisomer, a solvate, an N-oxide, or a pharmaceutically acceptable salt thereof: 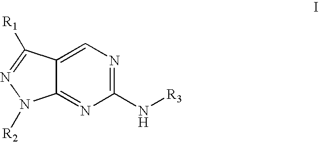
wherein:
R¹ is hydrogen or halo;
R² is -L₁-Rₐ, wherein
L₁ is a bond and Rₐ is alkenyl, alkynyl, cycloalkyl, heterocycloalkyl, aryl, or heteroaryl; in which each of cycloalkyl, heterocycloalkyl, aryl, and heteroaryl is optionally substituted and has 8 to 16 ring atoms; or
L₁ is alkyl and Rₐ is hydrogen, alkenyl, alkynyl, amino, hydroxy, alkoxycarbonyl, alkylcarbonyloxy, cycloalkyl, heterocycloalkyl, aryl, or heteroaryl; in which each of cycloalkyl, heterocycloalkyl, aryl, and heteroaryl is optionally substituted and has 8 to 16 ring atoms;
R³ is -R_{b}-L₂-R_{c}; wherein
R_{b} is aryl, heteroaryl, cycloalkyl, or heterocycloalkyl, and is optionally substituted with 1 to 3 substituents; wherein two of the substituents when adjacent, together with the atom or atoms to which they are attached, can form a 5- to 16-membered ring with 0 to 6 hetero ring atoms,
wherein R_{b} is optionally substituted with 1 to 3 substituents each independently selected from the group consisting of alkyl, alkenyl, alkynyl, cycloalkyl, heteroaryl, aryl, heteroaryl, arylalkyl, heteroarylalkyl, —OR, —SR, —NRR′, oxo, —C(O)—OR, —C(O)—NRR′, halo, —CN, —NO₂, —N₃, —C(O)R, —P(O)(OR)R′, —O—P(O)(OR)R′, —NR—P(O)(OR′)R″, —S(O)₂—(OR), —O—S(O)₂—(OR), —NR—S(O)₂—OR′, —NR—C(O)—OR′, —NR—C(O)—NR′R″, —NR—C(S)—NR′R″, —C(S)—NRR′, and thioalkyl;
wherein R is hydrogen, alkyl, alkenyl, alkynyl, cycloalkyl, heterocycloalkyl, cycloalkenyl, heterocycloalkenyl, aryl, heteroaryl, cycloalkyl-alkyl, heterocycloalkyl-alkyl, cycloalkenyl-alkyl, heterocycloalkenyl-alkyl, aralkyl, or heteroaralkyl; and, except for hydrogen, is optionally substituted with 1 to 3 substituents;
R_{c} is, except when being hydrogen, optionally substituted with 1 to 3 substituents each independently selected from the group consisting alkyl, alkenyl, alkynyl, heteroaryl, aryl, heteroaryl, arylalkyl, heteroarylalkyl, —OR, —SR, —NRR′, oxo, —C(O)—OR, —C(O)—NRR′, halo, —CN, —NO₂, —N₃, —C(O)R, —P(O)(OR)R′, —O—P(O)(OR)R′, —NR—P(O)(OR′)R″, —S(O)₂—(OR), —O—S(O)₂—(OR), —NR—S(O)₂—OR′, —NR—C(O)—OR′, —NR—C(O)—NR′R″, —NR—C(S)—NR′R″, —C(S)—NRR′, and thioalkyl;
each of R and R′, independently, is hydrogen, alkyl, cycloalkyl, heterocycloalkyl, aryl, heteroaryl, aralkyl, or heteroaralkyl; and
R″ is alkyl, cycloalkyl, heterocycloalkyl, aryl, heteroaryl, aralkyl, or heteroaralkyl,
L₂ is a bond, —(CRR)—, —N═, —O—, —S—, —SO—, —SO₂—, —CO—, —CO—O—, —O—CO—, —NRₓ—, —NRₓ—CO—, —NRₓ—SO₂—, —CO—NRₓ—, —SO₂—NRₓ—, —NRₓ—CO—NR_{y}—, —NRₓ—SO₂—NR_{y}—, —CO—NRₓ—NR_{y}—, —SO₂—NRₓ—NR_{y}—, —NRₓ—CO—CO—O—, —NRₓ—SO₂—SO₂—O—, —S(O)₂—Nₓ—CO—R_{y}—, —CO—Nₓ—S(O)₂—R—, or —(NRₓR_{y})C═N—O—;
each of Rₓ and R_{y}, independently, is hydrogen, hydroxy, alkyl, alkoxy, amino, —CO-alkyl, —CO-aryl, —SO₂-alkyl, —SO₂-aryl, —SO₂-heteroaryl, or —P(O)(O-alkyl)₂, wherein the alkyl or aryl moiety in Rₓ or R_{y} is optionally substituted with 1 to 3 substituents; —each independently selected from the group consisting of alkyl, alkenyl, alkynyl, cycloalkyl, heteroaryl, aryl, heteroaryl, arylalkyl, heteroarylalkyl, —OR, —SR, —NRR′, oxo, —C(O)—OR, —C(O)—NRR′, halo, —CN, —NO₂, —N₃, —C(O)R, —P(O)(OR)R′, —O—P(O)(OR)R′, —NR—P(O)(OR′)R″, —S(O)₂—(OR), —O—S(O)₂—(OR), —NR—S(O)₂—OR′, —NR—C(O)—OR′, —NR—C(O)—NR′R″, —NR—C(S)—NR′R″, —C(S)—NRR′, and thioalkyl;
and
n is 0, 1, 2, or 3.

57. The compound of claim 56, wherein R₃ is 4-bis(methanesulfonyl)aminophenyl, 4-bis(ethanesulfonyl)aminophenyl, 4-(4-methylpiperizin-1-yl)phenyl, 4-bis(methanesulfonyl)aminophenyl, 4-bis((chloromethane)sulfonyl)aminophenyl, 4-(methanesulfonyl)aminophenyl, 4-(N′-hydroxycarbamimidoyl)phenyl, 4-(4-methylpiperizin-1-yl)phenyl, 4-aminophenyl, 4-(methanesulfonylcarbamoyl)phenyl, 4-cyanophenyl, 4-(tetrazol-5-yl)aminophenyl, 2-(4-nitrophenyl)ethyl, 2-(4-aminophenyl)ethyl, or 2-(4-bis(methanesulfonyl)aminophenyl)ethyl.

58. The compound of claim 57, wherein Rₐ is cycloalkyl, heterocycloalkyl, aryl, and heteroaryl.

59. The compound of claim 58, wherein Rₐ is dihydroindenyl, tetrahydronaphthyl, dihydroacenaphthylenyl, naphthylenyl, tetrahydro-benzo[7]annulenyl, quinolinyl, isoquinolinyl, benzimidazolyl, benzothiazolyl, benzoxazolyl, phenanthrenyl, or tetrahydro-dibenzo[7]annulenyl; and Rₐ is optionally substituted with 1-3 substituents.

60. The compound of claim 59, wherein Rₐ is dihydroindenyl, tetrahydronaphthyl, dihydroacenaphthylenyl, or naphthyl; and Rₐ is optionally substituted with 1-3 substituents.

61. The compound of claim 60, wherein Rₐ is dihydroinden-1-yl, 4-hydroxydihydroinden-1-yl, 4-benzoyloxydihydroinden-1-yl, 4-dihydrophosphoryloxydihydroinden-1-yl, 5,6-dioxoledihydroinden-1-yl, 5-acetamidodihydroinden-1-yl, 5-benzoyloxydihydroinden-1-yl, 4-benzoyloxydihydroinden-1-yl, 4-(di-tert-butylphosphoryloxy)dihydroinden-1-yl, 54(2-methoxyethoxy)methyl)dihydroinden-1-yl, or 5-methoxydihydroinden-1-yl.

62. The compound of claim 58, wherein Rₐ is indolyl optionally substituted with 1 to 3 substituents.

63. The compound of claim 62, wherein Rₐ is indol-4-yl, indol-6-yl, indol-5-yl, 7-methylindol-5-yl, 1-tert-butylcarboxy-7-methylindol-5-yl, or (1-tert-butylcarboxy)indol-5-yl.

64. The compound of claim 63, wherein R₃ is 4-bis(methanesulfonyl)aminophenyl.

65. The compound of claim 64, wherein L₁ is methyl, ethyl, propyl, isopropyl, or butyl; and Rₐ is hydrogen, amino, hydroxy, alkoxycarbonyl, alkylcarbonyloxy, cycloalkyl, heterocycloalkyl, aryl, or heteroaryl; in which each of cycloalkyl, heterocycloalkyl, aryl, and heteroaryl is optionally substituted with 1-3 substituents.

66. The compound of claim 65, R₂ is naphthalen-1-ylmethyl.

67. The compound of claim 66, wherein R₃ is 4-(piperidin-1-yl)phenyl or 4-(bis(methylsulfonyl)amino)phenyl.

68. The compound of claim 56, wherein R_{b} is phenyl and is optionally substituted with 1 to 3 substituents; L₂ is —O—, —S—, —SO₂—, —CO—, —CO—O—, —NRₓ—, —NRₓ—CO—, —NRₓ—SO₂—, —NRₓ—CO—O—, —NRₓ—CO—NR_{y}—, or —NRₓ—CO—CO—O— wherein each of Rₓ and R_{y} is hydrogen, alkyl, —CO-alkyl, or —SO₂-alkyl.

69. The compound of claim 68, wherein R_{c} is hydrogen, alkyl, heteroalkyl or aryl.

70. The compound of claim 69, wherein L₂ is a bond; and R_{c} is cycloalkyl, heterocycloalkyl, aryl, heteroaryl, (cycloalkyl)alkyl, (heterocycloalkyl)alkyl, aralkyl, or heteroaralkyl.

71. The compound of claim 70, wherein L₂ is a bond and R_{c} is tetrazolyl, morpholino, or piperazinyl.

72. The compound of claim 56, wherein R_{c} is cycloalkyl, heterocycloalkyl, aryl, heteroaryl, cycloalkyl-alkyl, heterocycloalkyl-alkyl, aralkyl, or heteroaralkyl.

73. The compound of claim 72, wherein R_{b} is phenyl substituted with 1 to 3 substituents each independently selected from the group consisting of —NRR′ and —C(O)OR; L₂ is a bond; and R_{c} is hydrogen

74. The compound of claim 72, wherein R₁ is hydrogen.

75. The compound of claim 74, wherein L₁ is a bond; Rₐ is cycloalkyl, heterocycloalkyl, aryl, and heteroaryl; R_{b} is phenyl; L₂ is —O—, —S—, —SO₂—, —CO—, —CO—O—, —NRₓ—, —NRₓ—CO—, —NRₓ—SO₂—, —NRₓ—CO—O—, —NRₓ—CO—NR_{y}—, or —NRₓ—CO—CO—O— with Rₓ being hydrogen, alkyl, —CO-alkyl, —SO₂-alkyl, —SO₂-aryl, or —SO₂-heteroaryl; and R_{c} is hydrogen, alkyl, aryl, or heteroaryl.

76. The compound of claim 74, wherein L₁ is a bond; Rₐ is dihydroindenyl, tetrahydronaphthyl, dihydroacenaphthylenyl, or naphthyl; R_{b} is phenyl; L₂ is —CO—O—, —NRₓ—, —NRₓ—SO₂—, —NRₓ—CO—O—, or —NRₓ—CO—NR_{y}—, with Rₓ being hydrogen, alkyl, —CO-alkyl, —SO₂-alkyl, or —SO₂-aryl; and R_{c} is hydrogen, alkyl, or aryl.

77. The compound of claim 74, wherein L₁ is a bond; Rₐ is cycloalkyl, heterocycloalkyl, aryl, and heteroaryl; R_{b} is phenyl; L₂ is a bond; and R_{c} is cycloalkyl, heterocycloalkyl, aryl, heteroaryl, (cycloalkyl)alkyl, (heterocycloalkyl)alkyl, aralkyl, or heteroaralkyl.

78. The compound of claim 77, wherein L₁ is a bond; Rₐ is dihydroindenyl, tetrahydronaphthyl, dihydroacenaphthylenyl, or naphthyl; R_{b} is phenyl; L₂ is a bond; and R_{c} is tetrazolyl, morpholino, or piperazinyl.

79. The compound of claim 78, wherein L₁ is alkyl and Rₐ is hydrogen, alkenyl, alkynyl, amino, hydroxy, alkoxycarbonyl, alkylcarbonyloxy, cycloalkyl, heterocycloalkyl, aryl, or heteroaryl; in which each of cycloalkyl, heterocycloalkyl, aryl, and heteroaryl is optionally substituted with 1-3 substituents and has 8 to 16 ring atoms.

80. The compound of claim 74, wherein R_{b} is phenyl substituted with 1 to 3 substitutents each independently being —NRR′ or —C(O)OR; L₂ is a bond; and R_{c} is hydrogen.

81. The compound of claim 74, wherein L₂ is a bond or —NRₓ—SO₂— with Rₓ being hydrogen or
—SO₂-alkyl; and R_{c} is hydrogen, alkyl, heterocycloalkyl, heteroaryl, heterocycloalkyl-alkyl, or heteroaralkyl.

82. The compound of claim 74, wherein R_{b} is phenyl optionally substituted with —NRR′ or —C(O)OR; L₂ is a bond or —NRₓ—SO₂— with Rₓ being hydrogen or —SO₂-alkyl; and R_{c} is tetrazolyl, morpholino, or piperazinyl.

83. The compound of claim 74, wherein R₂ is 2,3-dihydro-1H-inden-1-yl, (S)-2,3-dihydro-1H-inden-1-yl, (R)-2,3-dihydro-1H-inden-1-yl, 1,2-dihydroacenaphthylen-1-yl, (R)-1,2-dihydroacenaphthylen-1-yl, (S)-1,2-dihydroacenaphthylen-1-yl, phenanthren-1-yl, or phenanthren-4-yl.

84. The compound of claim 74, wherein R₃ is 4-(1H-tetrazol-5-yl)phenyl, 3-hydroxy-4-methoxyphenyl, 4-(4-methylpiperazin-1-yl)phenyl, 4-(piperazin-1-yl)phenyl, 4-aminophenyl, 4-benzoic acid, 4-morpholinophenyl, 4-(N,N-dimethylsulfonamide)phenyl, or 4-(methanesulfonamide)phenyl.

85. The compound of claim 56, wherein the compound is
N-(methylsulfonyl)-N-(4-(1-(naphthalen-1-yl)-1H-pyrazolo[3,4-d]pyrimidin-6-ylamino)phen yl)methanesulfonamide,
N-methyl-N-(4-(1-(naphthalen-1-yl)-1H-pyrazolo[3,4-d]pyrimidin-6-ylamino)phenyl)methanesulfonamide,
N-methyl-N-(4-(1-(naphthalen-1-yl)-1H-pyrazolo[3,4-d]pyrimidin-6-ylamino)phen yl)methanesulfonamide,
1-(methylsulfonyl)-N-(4-(1-(naphthalen-1-yl)-1H-pyrazolo[3,4-d]pyrimidin-6-ylamino)phenyl)methanesulfonamide,
N¹-(1-(naphthalen-2-yl)-1H-pyrazolo[3,4-d]pyrimidin-6-yl)benzene-1,4-diamine,
ethyl-2-(4-(1-(naphthalen-1-yl)-1H-pyrazolo[3,4-d]pyrimidin-6-yl amino)phenylamino)-2-oxoacetate,
N-(4-(1-(naphthalen-2-yl)-1H-pyrazolo[3,4-d]pyrimidin-6-ylamino)phen yl)methanesulfonamide,
3,3,3-trifluoro-N-(4-(1-(naphthalen-1-yl)-1H-pyrazolo[3,4-d]pyrimidin-6-ylamino)phenyl)propane-1-sulfonamide,
methyl-4-(1-(naphthalen-1-yl)-1H-pyrazolo[3,4-d]pyrimidin-6-ylamino)phenylcarbamate,
N-(3,4-dimethoxyphenyl)-1-(naphthalen-1-yl)-1H-pyrazolo[3,4-d]pyrimidin-6-amine,
(1-10 above)
N-(4-(4-methylpiperazin-1-yl)phenyl)-1-(naphthalen-2-yl)-1H-pyrazolo[3,4-d]pyrimidin-6-amine,
ethyl-2-hydroxy-4-(1-(naphthalen-1-yl)-1H-pyrazolo[3,4-d]pyrimidin-6-yl amino)phenylcarbamate,
N-(4-(1H-1,2,4-triazol-1-yl)phen yl)-1-(naphthalen-1-yl)-1H-pyrazolo[3,4-d]pyrimidin-6-amine,
methyl3-(4-(1-(naphthalen-1-yl)-1H-pyrazolo[3,4-d]pyrimidin-6-ylamino)phenylamino)-3-oxopropanoate,
N¹-(1-(naphthalen-1-yl)-1H-pyrazolo[3,4-d]pyrimidin-6-yl)benzene-1,4-diamine,
N-(4-(4-methylpiperazin-1-yl)phen yl)-1-(naphthalen-1-yl)-1H-pyrazolo[3,4-d]pyrimidin-6-amine,
N-(4-fluoro-3-methoxyphenyl)-1-(naphthalen-1-yl)-1H-pyrazolo[3,4-d]pyrimidin-6-amine,
N-(4-ethoxyphenyl)-1-(naphthalen-1-yl)-1H-pyrazolo[3,4-d]pyrimidin-6-amine,
(S)-1-(2,3-dihydro-1H-inden-1-yl)-N-(4-(4-methylpiperazin-1-yl)phenyl)-1H-pyrazolo[3,4-d]pyrimidin-6-amine,
(S)-4-(1-(2,3-dihydro-1H-inden-1-yl)-1H-pyrazolo[3,4-d]pyrimidin-6-ylamino)benzoic acid,
(11-20 above)
(S)—N-(4-(1-(2,3-dihydro-1H-inden-1-yl)-1H-pyrazolo[3,4-d]pyrimidin-6-ylamino)phenyl)-N-(methylsulfonyl)methanesulfonamide,
(S)—N¹-(1-(2,3-dihydro-1H-inden-1-yl)-1H-pyrazolo[3,4-d]pyrimidin-6-yl)benzene-1,4-diamine,
(S)-1-(2,3-dihydro-1H-inden-1-yl)-N-(4-morpholinophenyl)-1H-pyrazolo[3,4-d]pyrimidin-6-amine,
(S)-1-(2,3-dihydro-1H-inden-1-yl)-N-(4-(4-(propylsulfonyl)piperazin-1-yl)phenyl)-1H-pyrazolo[3,4-d]pyrimidin-6-amine,
4-(1-(2,3-dihydro-1H-inden-1-yl)-1H-pyrazolo[3,4-d]pyrimidin-6-ylamino)benzoic acid,
1-(2,3-dihydro-1H-inden-1-yl)-N-(3-(piperazin-1-yl)phenyl)-1H-pyrazolo[3,4-d]pyrimidin-6-amine,
N¹-(1-(2,3-dihydro-1H-inden-1-yl)-1H-pyrazolo[3,4-d]pyrimidin-6-yl)benzene-1,4-diamine,
N-(4-(1-(2,3-dihydro-1H-inden-1-yl)-1H-pyrazolo[3,4-d]pyrimidin-6-ylamino)phenyl)methanesulfonamide,
1-(2,3-dihydro-1H-inden-1-yl)-N-(4-(4-(propylsulfonyl)piperazin-1-yl)phenyl)-1H-pyrazolo[3,4-d]pyrimidin-6-amine,
(S)-1-(2,3-dihydro-1H-inden-1-yl)-N-(4-(4-(methylsulfonyl)piperazin-1-yl)phenyl)-1H-pyrazolo[3,4-d]pyrimidin-6-amine,
(21-30 above)
(S)-1-(2,3-dihydro-1H-inden-1-yl)-N-(4-(piperazin-1-yl)phenyl)-1H-pyrazolo[3,4-d]pyrimidin-6-amine,
N-(4-(1-(2,3-dihydro-1H-inden-1-yl)-1H-pyrazolo[3,4-d]pyrimidin-6-ylamino)phenyl)benzamide,
(S)-5-(1-(2,3-dihydro-1H-inden-1-yl)-1H-pyrazolo[3,4-d]pyrimidin-6-ylamino)-2-methoxyphenol,
1-(2,3-dihydro-1H-inden-1-yl)-N-(4-(piperazin-1-yl)phenyl)-1H-pyrazolo[3,4-d]pyrimidin-6-amine,
1-(2,3-dihydro-1H-inden-1-yl)-N-(4-morpholinophenyl)-1H-pyrazolo[3,4-d]pyrimidin-6-amine,
3-(4-(4-(1-(2,3-dihydro-1H-inden-1-yl)-1H-pyrazolo[3,4-d]pyrimidin-6-ylamino)phenyl)piperazin-1-yl)propan-1-o|″
4-(1-(2,3-dihydro-1H-inden-1-yl)-1H-pyrazolo[3,4-d]pyrimidin-6-ylamino)benzohydrazide,
2-(4-(4-(1-(2,3-dihydro-1H-inden-1-yl)-1H-pyrazolo[3,4-d]pyrimidin-6-ylamino)phenyl)piperazin-1-yl)ethylacetate,
1-(2,3-dihydro-1H-inden-1-yl)-N-(4-(44(4-methoxy-3,5-dimethylpyridin-2-yl)methyl)piperazin-1-yl)phenyl)-1H-pyrazolo[3,4-d]pyrimidin-6-amine,
3-(4-(4-(1-(2,3-dihydro-1H-inden-1-yl)-1H-pyrazolo[3,4-d]pyrimidin-6-ylamino)phenyl)piperazin-1-yl)propylacetate,
(31-40 above)
2-(4-(4-(1-(2,3-dihydro-1H-inden-1-yl)-1H-pyrazolo[3,4-d]pyrimidin-6-ylamino)phenyl)piperazin-1-yl)ethanol,
1-((S)-2,3-dihydro-1H-inden-1-yl)-N-(3-(14(R)-2,3-dihydro-1H-inden-1-yl)-1H-pyrazolo[3,4-d]pyrimidin-6-yloxy)-4-methoxyphenyl)-1H-pyrazolo[3,4-d]pyrimidin-6-amine,
(S)-ethyl-4-(1-(2,3-dihydro-1H-inden-1-yl)-1H-pyrazolo[3,4-d]pyrimidin-6-ylamino)benzoate,
(S)-1-(2,3-dihydro-1H-inden-1-yl)-N-(4-morpholinophenyl)-1H-pyrazolo[3,4d]pyrimidin-6-amine,
N-(4-(1-(4-hydroxy-2,3-dihydro-1H-inden-1-yl)-1H-pyrazolo[3,4-d]pyrimidin-6-ylamino)phenyl)-N-(methylsulfonyl)methanesulfonamide,
(S)—N-(4-(1-(2,3-dihydro-1H-inden-1-yl)-1H-pyrazolo[3,4-d]pyrimidin-6-ylamino)phenyl)-N-(ethylsulfonyl)ethanesulfonamide,
1-(6-(4-(4-methylpiperazin-1-yl)phenylamino)-1H-pyrazolo[3,4-d]pyrimidin-1-yl)-2,3-dihydro-1H-inden-4-ol,
1-(6-(4-(N-(methylsulfonyl)methylsulfonamido)phenylamino)-1H-pyrazolo[3,4-d]pyrimidin-1-yl)-2,3-dihydro-1H-inden-4-yl benzoate,
1-(6-(4-(N-(methylsulfonyl)methylsulfonamido)phenylamino)-1H-pyrazolo[3,4-d]pyrimidin-1-yl)-2,3-dihydro-1H-inden-4-yl dihydrogen phosphate,
(S)-1-chloro-N-(chloromethylsulfonyl)-N-(4-(1-(2,3-dihydro-1H-inden-1-yl)-1H-pyrazolo[3,4-d]pyrimidin-6-ylamino)phenyl)methanesulfonamide,
(41-50 above)
N-(4-(1-(6,7-dihydro-5H-indeno[5,6-d][1,3]dioxol-5-yl)-1H-pyrazolo[3,4-d]pyrimidin-6-ylamino)phenyl)-N-(methylsulfonyl)methanesulfonamide,
N-(1-(6-(4-(N-(methylsulfonyl)methylsulfonamido)phenylamino)-1H-pyrazolo[3,4-d]pyrimidin-1-yl)-2,3-dihydro-1H-inden-5-yl)acetamide,
N-(4-(1-(4-hydroxy-2,3-dihydro-1H-inden-1-yl)-1H-pyrazolo[3,4-d]pyrimidin-6-ylamino)phenyl)methanesulfonamide,
1-(6-(4-(N-(methylsulfonyl)methylsulfonamido)phenylamino)-1H-pyrazolo[3,4-d]pyrimidin-1-yl)-2,3-dihydro-1H-inden-5-yl benzoate,
(S,Z)-4-(1-(2,3-dihydro-1H-inden-1-yl)-1H-pyrazolo[3,4-d]pyrimidin-6-ylamino)-N′-hydroxybenzimidamide,
1-(6-(4-(4-methylpiperazin-1-yl)phenylamino)-1H-pyrazolo[3,4-d]pyrimidin-1-yl)-2,3-dihydro-1H-inden-4-yl benzoate,
1-(6-(4-aminophenylamino)-1H-pyrazolo[3,4-d]pyrimidin-1-yl)-2,3-dihydro-1H-inden-4-ol,
1-(6-(4-aminophenylamino)-1H-pyrazolo[3,4-d]pyrimidin-1-yl)-2,3-dihydro-1H-inden-4-yl benzoate,
di-tert-butyl 1-(6-(4-(N-(methylsulfonyl)methylsulfonamido)phenylamino)-1H-pyrazolo[3,4-d]pyrimidin-1-yl)-2,3-dihydro-1H-inden-4-yl phosphate,
(S)-4-(1-(2,3-dihydro-1H-inden-1-yl)-1H-pyrazolo[3,4-d]pyrimidin-6-ylamino)-N-(methylsulfonyl)benzamide,
(51-60 above)
N-(4-(1-(5-((2-methoxyethoxy)methoxy)-2,3-dihydro-1H-inden-1-yl)-1H-pyrazolo[3,4-d]pyrimidin-6-ylamino)phenyl)-N-(methylsulfonyl)methanesulfonamide,
N-(1-(6-(4-aminophenylamino)-1H-pyrazolo[3,4-d]pyrimidin-1-yl)-2,3-dihydro-1H-inden-5-yl)acetamide,
(S)-4-(1-(2,3-dihydro-1H-inden-1-yl)-1H-pyrazolo[3,4-d]pyrimidin-6-ylamino)benzonitrile,
(S)—N-(4-(1H-tetrazol-5-yl)phenyl)-1-(2,3-dihydro-1H-inden-1-yl)-1H-pyrazolo[3,4-d]pyrimidin-6-amine,
N¹-(1-(5-methoxy-2,3-dihydro-1H-inden-1-yl)-1H-pyrazolo[3,4-d]pyrimidin-6-yl)benzene-1,4-diamine,
N-(4-(1-(5-methoxy-2,3-dihydro-1H-inden-1-yl)-1H-pyrazolo[3,4-d]pyrimidin-6-ylamino)phenyl)-N-(methylsulfonyl)methanesulfonamide,
1-(2,3-dihydro-1H-inden-2-yl)-N-(4-morpholinophenyl)-1H-pyrazolo[3,4-d]pyrimidin-6-amine,
N-(4-(1-(1H-indol-4-yl)-1H-pyrazolo[3,4-d]pyrimidin-6-ylamino)phenyl)-N-(methylsulfonyl)methanesulfonamide,
N-(4-(1-(1H-indol-6-yl)-1H-pyrazolo[3,4-d]pyrimidin-6-ylamino)phenyl)-N-(methylsulfonyl)methanesulfonamide,
N-(4-(1-(1H-indol-5-yl)-1H-pyrazolo[3,4-d]pyrimidin-6-ylamino)phenyl)-N-(methylsulfonyl)methanesulfonamide,
(61-70 above)
1-(1H-indol-4-yl)-N-(4-(4-methylpiperazin-1-yl)phenyl)-1H-pyrazolo[3,4-d]pyrimidin-6-amine,
N-(4-(1-(1-methyl-1H-indol-5-yl)-1H-pyrazolo[3,4-d]pyrimidin-6-ylamino)phenyl)-N-(methylsulfonyl)methanesulfonamide,
1-(7-methyl-1H-indol-4-yl)-N-(4-(4-methylpiperazin-1-yl)phenyl)-1H-pyrazolo[3,4-d]pyrimidin-6-amine,
tert-butyl 7-methyl-4-(6-(4-(4-methylpiperazin-1-yl)phenylamino)-1H-pyrazolo[3,4-d]pyrimidin-1-yl)-1H-indole-1-carboxylate,
tert-butyl 4-(6-(4-(4-methylpiperazin-1-yl)phenylamino)-1H-pyrazolo[3,4-d]pyrimidin-1-yl)-1H-indole-1-carboxylate
1-(1,2-dihydroacenaphthylen-1-yl)-N-(4-(4-methylpiperazin-1-yl)phenyl)-1H-pyrazolo[3,4-d]pyrimidin-6-amine,
N-(4-(1-(1,2-dihydroacenaphthylen-1-yl)-1H-pyrazolo[3,4-d]pyrimidin-6-ylamino)phenyl)-N-(methylsulfonyl)methanesulfonamide,
N¹-(1-(1,2-dihydroacenaphthylen-1-yl)-1H-pyrazolo[3,4-d]pyrimidin-6-yl)benzene-1,4-diamine,
1-(1,2-dihydroacenaphthylen-1-yl)-N-(4-morpholinophenyl)-1H-pyrazolo[3,4-d]pyrimidin-6-amine,
4-(1-(1,2-dihydroacenaphthylen-1-yl)-1H-pyrazolo[3,4-d]pyrimidin-6-ylamino)benzonitrile,
(71-80 above)
1-(1,2-dihydroacenaphthylen-1-yl)-N-(4-(2-(trimethylstannyl)-2H-tetrazol-5-yl)phenyl)-1H-pyrazolo[3,4-d]pyrimidin-6-amine,
1-(1,2-dihydroacenaphthylen-1-yl)-N-(4-(piperazin-1-yl)phenyl)-1H-pyrazolo[3,4-d]pyrimidin-6-amine,
N-(4-morpholinophenyl)-1-(naphthalen-1-ylmethyl)-1H-pyrazolo[3,4-d]pyrimidin-6-amine, or
N-(4-morpholinophenyl)-1-(naphthalen-1-ylmethyl)-1H-pyrazolo[3,4-d]pyrimidin-6-amine.

86. The compound of claim 56, wherein the compound is
N-(4-(1H-tetrazol-5-yl)phenyl)-1-((S)-(2,3-dihydro-1H-inden-1-yl))-1H-pyrazolo[3,4-d]pyrimidin-6-amine,
N-(4-(1H-tetrazol-5-yl)phenyl)-1-((R)-(2,3-dihydro-1H-inden-1-yl))-1H-pyrazolo[3,4-d]pyrimidin-6-amine,
N-(4-(1H-tetrazol-5-yl)phenyl)-1-(2,3-dihydro-1H-inden-1-yl)-1H-pyrazolo[3,4d]-pyrimidin-6-amine,
N-(4-(1H-tetrazol-5-yl)phenyl)-1-((S)-(1,2-dihydroacenaphthylen-1-yl))-1H-pyrazolo[3,4-d]pyrimidin-6-amine,
N-(4-(1H-tetrazol-5-yl)phen yl)-1-((R)-(1,2-dihydroacenaphthylen-1-yl))-1H-pyrazolo[3,4-d]pyrimidin-6-amine,
N-(4-(1H-tetrazol-5-yl)phenyl)-1-(1,2-dihydroacenaphthylen-1-yl)-1H-pyrazolo[3,4-d]pyrimidin-6-amine,
N-(4-(1H-tetrazol-5-yl)phenyl)-1-(1-naphthyl)-1H-pyrazolo[3,4-d]pyrimidin-6-amine,
N-(4-(1H-tetrazol-5-yl)phenyl)-1-(phenanthren-4-yl)-1H-pyrazolo[3,4-d]pyrimidin-6-amine,
N-(4-morpholinophenyl)-14(S)-(2,3-dihydro-1H-inden-1-yl))-1H-pyrazolo[3,4-d]pyrimidin-6-amine,
N-(4-morpholinophenyl)-14(R)-(2,3-dihydro-1H-inden-1-yl))-1H-pyrazolo[3,4-d]-pyrimidin-6-amine,
(1-10 above)
N-(4-morpholinophenyl)-1-(2,3-dihydro-1H-inden-1-yl)-1H-pyrazolo[3,4-d]pyrimidin-6-amine,
N-(4-morpholinophenyl)-1-((S)-(1,2-dihydroacenaphthylen-1-yl))-1H-pyrazolo[3,4-d]pyrimidin-6-amine,
N-(4-morpholinophenyl)-1-((R)-(1,2-dihydroacenaphthylen-1-yl))-1H-pyrazolo[3,4-d]pyrimidin-6-amine,
N-(4-morpholinophenyl)-1-(1,2-dihydroacenaphthylen-1-yl)-1H-pyrazolo[3,4-d]pyrimidin-6-amine,
N-(4-morpholinophenyl)-1-(1-naphthyl)-1H-pyrazolo[3,4-d]pyrimidin-6-amine,
N-(4-morpholinophenyl)-1-(phenanthren-4-yl)-11/-pyrazolo[3,4-d]-pyrimidin-6-amine,
N-(4-(piperazin-1-yl)phenyl)-1-((S)-(2,3-dihydro-1H-inden-1-yl))-1H-pyrazolo[3,4-d]pyrimidin-6-amine,
N-(4-(piperazin-1-yl)phenyl)-1-((R)-(2,3-dihydro-1H-inden-1-yl))-1H-pyrazolo[3,4-d]pyrimidin-6-amine,
N-(4-(piperazin-1-yl)phenyl)-1-(2,3-dihydro-1H-inden-1-yl)-1H-pyrazolo[3,4-d]pyrimidin-6-amine,
N-(4-(piperazin-1-yl)phenyl)-1-((S)-(1,2-dihydroacenaphthylen-1-yl))-1H-pyrazolo[3,4-d]pyrimidin-6-amine,
(11-20 above)
N-(4-(piperazin-1-yl)phen yl)-1-((R)-(1,2-dihydroacenaphthylen-1-yl))-1H-pyrazolo[3,4-d]pyrimidin-6-amine,
N-(4-(piperazin-1-yl)phenyl)-1-(1,2-dihydroacenaphthylen-1-yl)-1H-pyrazolo[3,4-d]-pyrimidin-6-amine,
N-(4-(piperazin-1-yl)phenyl)-1-(1-naphthyl)-1H-pyrazolo[3,4-d]pyrimidin-6-amine,
N-(4-(piperazin-1-yl)phenyl)-1-(phenanthren-4-yl)-1H-pyrazolo[3,4-d]pyrimidin-6-amine,
(S)-4-(1-(2,3-dihydro-1H-inden-1-yl)-1H-pyrazolo[3,4-d]pyrimidin-6-ylamino)benzoic acid,
(R)-4-(1-(2,3-dihydro-1H-inden-1-yl)-1H-pyrazolo[3,4-d]pyrimidin-6-ylamino)benzoic acid,
4-(1-(2,3-dihydro-1H-inden-1-yl)-1H-pyrazolo[3,4-d]pyrimidin-6-ylamino)benzoic acid,
(S)-4-(1-(1,2-dihydroacenaphthylen-1-yl)-1H-pyrazolo[3,4-d]pyrimidin-6-ylamino)benzoic acid,
(R)-4-(1-(1,2-dihydroacenaphthylen-1-yl)-1H-pyrazolo[3,4-d]pyrimidin-6-ylamino)benzoic acid,
4-(1-(1,2-dihydroacenaphthylen-1-yl)-1H-pyrazolo[3,4-d]pyrimidin-6-ylamino)benzoic acid,
(21-30 above)
4-(1-(1-napthyl)-1H-pyrazolo[3,4-d]pyrimidin-6-ylamino)benzoic acid,
4-(1-(phenanthren-4-yl)-1H-pyrazolo[3,4-d]pyrimidin-6-ylamino)benzoic acid,
N-(4-aminophenyl)-14(S)-(2,3-dihydro-1H-inden-1-yl))-1H-pyrazolo[3,4-d]pyrimidin-6-amine,
N-(4-aminophen yl)-1-((R)-(2,3-dihydro-1H-inden-1-yl))-1H-pyrazolo[3, 4-d]pyrimidin-6-amine,
N-(4-aminophenyl)-1-(2,3-dihydro-1H-inden-1-yl)-1H-pyrazolo[3,4-d]pyrimidin-6-amine,
N-(4-aminophenyl)-14(S)-(1,2-dihydroacenaphthylen-1-yl))-1H-pyrazolo[3,4-d]pyrimidin-6-amine,
N-(4-aminophenyl)-14(R)-(1,2-dihydroacenaphthylen-1-yl))-1H-pyrazolo[3,4-d]pyrimidin-6-amine,
N-(4-aminophenyl)-1-(1,2-dihydroacenaphthylen-1-yl)-1H-pyrazolo[3,4-c]pyrimidin-6-amine,
N-(4-aminophenyl)-1-(1-naphthyl)-1H-pyrazolo[3,4-c]pyrimidin-6-amine,
N-(4-aminophenyl)-1-(phenanthren-4-yl)-1H-pyrazolo[3,4-d]pyrimidin-6-amine,
(31-40 above)
(S)—N-(4-(1-(2,3-dihydro-1H-inden-1-yl)-1H-pyrazolo[3,4-d]pyrimidin-6-ylamino)phenyl)-N-(methylsulfonyl)methanesulfonamide,
(R)—N-(4-(1-(2,3-dihydro-1H-inden-1-yl)-1H-pyrazolo[3,4-d]pyrimidin-6-ylamino)phenyl)-N-(methylsulfonyl)methanesulfonamide,
N-(Methylsulfonyl)-N-(4-(1-(2,3-dihydro-1H-inden-1-yl)-1H-pyrazolo[3,4-d]pyrimidin-6-ylamino)phenyl)methanesulfonamide,
(S)—N-(4-(1-(1,2-dihydroacenaphthylen-1-yl)-1H-pyrazolo[3,4-d]pyrimidin-6-ylamino)phenyl)-N-(methylsulfonyl)methanesulfonamide,
(R)—N-(4-(1-(1,2-dihydroacenaphthylen-1-yl)-/H-pyrazolo[3,4-d]pyrimidin-6-ylamino)phenyl)-N-(methylsulfonyl)methanesulfonamide,
N-(4-(1-(1,2-dihydroacenaphthylen-1-yl)-1H-pyrazolo[3,4-d]pyrimidin-6-ylamino)phenyl)-N-(methylsulfonyl)methanesulfonamide,
N-(4-(1-(1-naphthyl)-1H-pyrazolo[3,4-d]pyrimidin-6-ylamino)phenyl)-N-(methylsulfonyl)methanesulfonamide,
N-(4-(1-(phenanthren-4-yl)-1H-pyrazolo[3,4-d]pyrimidin-6-ylamino)phenyl)-N-(methylsulfonyl)methanesulfonamide,
(S)—N-(4-(1-(2,3-dihydro-1H-inden-1-yl)-1H-pyrazolo[3,4-d]pyrimidin-6-ylamino)phenyl)methanesulfonamide,
(R)—N-(4-(1-(2,3-dihydro-1H-inden-1-yl)-1H-pyrazolo[3,4-d]-pyrimidin-6-ylamino)phenyl)methanesulfonamide,
(41-50 above)
N-(4-(1-(2,3-dihydro-1H-inden-1-yl)-1H-pyrazolo[3,4-d]pyrimidin-6-ylamino)phenyl)methanesulfonamide,
(S)—N-(4-(1-(1,2-dihydroacenaphthylen-1-yl)-/H-pyrazolo[3,4-d]pyrimidin-6-ylamino)phenyl)methanesulfonamide,
(R)—N-(4-(1-(1,2-dihydroacenaphthylen-1-yl)-/H-pyrazolo[3,4-d]-pyrimidin-6-ylamino)phenyl)methanesulfonamide,
N-(4-(1-(1,2-dihydroacenaphthylen-1-yl)-/H-pyrazolo[3,4-d]pyrimidin-6-ylamino)phenyl)methanesulfonamide,
N-(4-(1-(1-naphthyl)-/H-pyrazolo[3,4-d]pyrimidin-6-ylamino)phenyl)methanesulfonamide,
N-(4-(1-(phenanthren-4-yl)-1H-pyrazolo[3,4-d]pyrimidin-6-ylamino)phenyl)methanesulfonamide,
N-4-(4-methylpiperazin-1-yl)phen yl-1-(S)-(2,3-dihydro-1H-inden-1-yl)-1H-pyrazolo[3,4-d]pyrimidin-6-amine,
N-4-(4-methylpiperazin-1-yl)phenyl-1-(R)-(2,3-dihydro-1H-inden-1-yl)-1H-pyrazolo[3,4-d]pyrimidin-6-amine,
N-4-(4-methylpiperazin-1-yl)phenyl-1-(2,3-dihydro-1H-inden-1-yl)-1H-pyrazolo[3,4-d]pyrimidin-6-amine,
N-4-(4-methylpiperazin-1-yl)phenyl-1-(S)-(1,2-dihydroacenaphthylen-1-yl)-1H-pyrazolo[3, 4-d]pyrimidin-6-amine,
(51-60 above)
N-4-(4-methylpiperazin-1-yl)phenyl-1-(R)-(1,2-dihydroacenaphthylen-1-yl)-1H-pyrazolo[3,4-d]pyrimidin-6-amine,
N-4-(4-methylpiperazin-1-yl)phenyl-1-(1,2-dihydroacenaphthylen-1-yl)-1H-pyrazolo[3,4-d]pyrimidin-6-amine,
N-4-(4-methylpiperazin-1-yl)phenyl-1-(1-naphthyl)-1H-pyrazolo[3,4-d]pyrimidin-6-amine,
N-(4-(1-(phenanthren-4-yl)-/H-pyrazolo[3,4-d]pyrimidin-6-ylamino) phenyl)methanesulfonamide,
N-(3-hydroxy-4-methoxyphenyl)-1-((S)-(2,3-dihydro-1H-inden-1-yl))-1H-pyrazolo[3,4-d]pyrimidin-6-amine,
N-(3-hydroxy-4-methoxyphenyl)-1-((R)-(2,3-dihydro-1H-inden-1-yl))-1H-pyrazolo[3,4-d]pyrimidin-6-amine,
N-(3-hydroxy-4-methoxyphenyl)-1-(2,3-dihydro-1H-inden-1-yl)-1H-pyrazolo[3,4-d]pyrimidin-6-amine,
N-(3-hydroxy-4-methoxyphenyl)-14(S)-(1,2-dihydroacenaphthylen-1-yl))-1H-pyrazolo[3,4-d]pyrimidin-6-amine,
N-(3-hydroxy-4-methoxyphenyl)-1-((R)-(1,2-dihydroacenaphthylen-1-yl))-1H-pyrazolo[3,4-d]-pyrimidin-6-amine,
N-(3-hydroxy-4-methoxyphenyl)-1-(1,2-dihydroacenaphthylen-1-yl)-1H-pyrazolo[3,4-d]pyrimidin-6-amine,
(61-70 above)
N-(3-hydroxy-4-methoxyphenyl)-1-(1-naphthyl)-1H-pyrazolo[3,4-d]-pyrimidin-6-amine,
N-(3-hydroxy-4-methoxyphenyl)-1-(phenanthren-4-yl)-1H-pyrazolo[3,4-d]-pyrimidin-6-amine,
N-(4-(1-(4-(benzyloxy)-5,6,7,8-tetrahydronaphthalen-1-yl)-1H-pyrazolo[3,4-d]pyrimidin-6-ylamino)phenyl)-N-(methylsulfonyl)methanesulfonamide,
1-(4-(benzyloxy)-5,6,7,8-tetrahydronaphthalen-1-yl)-N-(4-(4-methylpiperazin-1-yl)phenyl)-1H-pyrazolo[3,4-d]pyrimidin-6-amine,
N1-(1-(4-(benzyloxy)-5,6,7,8-tetrahydronaphthalen-1-yl)-1H-pyrazolo[3,4-d]pyrimidin-6-yl)benzene-1,4-diamine,
N-(4-morpholinophenyl)-1-(1,2,3,4-tetrahydronaphthalen-1-yl)-1H-pyrazolo[3,4-d]pyrimidin-6-amine,
4-(6-(4-aminophenylamino)-1H-pyrazolo[3,4-d]pyrimidin-1-yl)-5,6,7,8-tetrahydronaphthalen-1-01,
tert-butyl 4-(1-(4-(benzyloxy)-5,6,7,8-tetrahydronaphthalen-1-yl)-1H-pyrazolo[3,4-d]pyrimidin-6-ylamino)piperidine-1-carboxylate,
4-(6-(4-(N-(methylsulfonyl)methylsulfonamido)phenylamino)-1H-pyrazolo[3,4-d]pyrimidin-1-yl)-5,6,7,8-tetrahydronaphthalen-1-yl methanesulfonate,
4-(6-(4-(methylsulfonamido)phenylamino)-1H-pyrazolo[3,4-d]pyrimidin-1-yl)-5,6,7,8-tetrahydronaphthalen-1-yl methanesulfonate,
(71-80 above)
N-(4-(1-(4-(benzyloxy)-5,6,7,8-tetrahydronaphthalen-1-yl)-1H-pyrazolo[3,4-d]pyrimidin-6-ylamino)phenyl)methanesulfonamide,
N-(4-(1-(4-hydroxy-5,6,7,8-tetrahydronaphthalen-1-yl)-1H-pyrazolo[3,4-d]pyrimidin-6-ylamino)phenyl)-N-(methylsulfonyl)methanesulfonamide,
N-(4-(1-(4-hydroxy-5,6,7,8-tetrahydronaphthalen-1-yl)-1H-pyrazolo[3,4-d]pyrimidin-6-ylamino)phenyl)methanesulfonamide,
4-(6-(4-(4-methylpiperazin-1-yl)phenylamino)-1H-pyrazolo[3,4-d]pyrimidin-1-yl)-5,6,7,8-tetrahydronaphthalen-1-ol,
1-(4-(benzyloxy)-5,6,7,8-tetrahydronaphthalen-1-yl)-N-(4-morpholinophenyl)-1H-pyrazolo[3,4-d]pyrimidin-6-amine,
4-(6-(4-morpholinophenylamino)-1H-pyrazolo[3,4-d]pyrimidin-1-yl)-5,6,7,8-tetrahydronaphthalen-1-ol,
1-((6-fluoro-4H-benzo[d][1,3]dioxin-8-yl)methyl)-N-(4-morpholinophenyl)-1H-pyrazolo[3,4-d]pyrimidin-6-amine,
1-butyl-N-(4-(piperidin-1-yl)phenyl)-1H-pyrazolo[3,4-d]pyrimidin-6-amine,
ethyl 2-(6-(4-(piperidin-1-yl)phenylamino)-1H-pyrazolo[3,4-d]pyrimidin-1-yl)acetate,
1-methyl-N-(4-(piperidin-1-yl)phenyl)-1H-pyrazolo[3,4-d]pyrimidin-6-amine,
(81-90 above)
1-isopropyl-N-(4-(piperidin-1-yl)phenyl)-1H-pyrazolo[3,4-d]pyrimidin-6-amine,
N-(4-(1-(3-aminopropyl)-1H-pyrazolo[3,4-d]pyrimidin-6-ylamino)phenyl)-N-(methylsulfonyl)methanesulfonamide,
N-(methylsulfonyl)-N-(4-(1-propyl-1H-pyrazolo[3,4-d]pyrimidin-6-ylamino)phenyl)methanesulfonamide,
N-(4-(1-butyl-1H-pyrazolo[3,4-d]pyrimidin-6-ylamino)phenyl)-N-(methylsulfonyl)methanesulfonamide,
N-(4-(1-(4-hydroxybutyl)-1H-pyrazolo[3,4-d]pyrimidin-6-ylamino)phenyl)-N-(methylsulfonyl)methanesulfonamide,
4-(6-(4-(N-(methylsulfonyl)methylsulfonamido)phenylamino)-1H-pyrazolo[3,4-d]pyrimidin-1-yl)butyl acetate,
N-(4-(1-ethyl-1H-pyrazolo[3,4-d]pyrimidin-6-ylamino)phenyl)-N-(methylsulfonyl)methanesulfonamide,
N-(4-(1-isopropyl-1H-pyrazolo[3,4-d]pyrimidin-6-ylamino)phenyl)-N-(methylsulfonyl)methanesulfonamide, or
N-(4-(1-methyl-1H-pyrazolo[3,4-d]pyrimidin-6-ylamino)phenyl)-N-(methylsulfonyl)methanesulfonamide.
(99 last)

87. The compound of claim 56, wherein the compound is
N-(5-(1-(4-hydroxy-2,3-dihydro-1H-inden-1-yl)-1H-pyrazolo[3,4-d]pyrimidin-6-ylamino)pyridin-2-yl)methanesulfonamide,
N-(2,3-dihydroxypropyl)-N-(5-(1-(4-hydroxy-2,3-dihydro-1H-inden-1-yl)-1H-pyrazolo[3,4-d]pyrimidin-6-ylamino)pyridin-2-yl)methanesulfonamide,
N-(2,3-dihydroxypropyl)-N-(4-(1-(4-hydroxy-2,3-dihydro-1H-inden-1-yl)-1H-pyrazolo[3,4-d]pyrimidin-6-ylamino)phenyl)methanesulfonamide,
N-(5-(1-(4-hydroxy-2,3-dihydro-1H-inden-1-yl)-1H-pyrazolo[3,4-d]pyrimidin-6-ylamino)pyridin-2-yl)-N-(2-morpholinoethyl)methanesulfonamide,
N-(4-(1-(4-hydroxy-2,3-dihydro-1H-inden-1-yl)-1H-pyrazolo[3,4-d]pyrimidin-6-ylamino)phenyl)-N-(2-morpholinoethyl)methanesulfonamide,
N-(4-(1-(4-hydroxy-2,3-dihydro-1H-inden-1-yl)-1H-pyrazolo[3,4-d]pyrimidin-6-ylamino)phenyl)-N-(3-hydroxypropyl)methanesulfonamide,
N-(4-(1-(4-hydroxy-2,3-dihydro-1H-inden-1-yl)-1H-pyrazolo[3,4-d]pyrimidin-6-ylamino)phenyl)N-(2-hydroxyethyl)methanesulfonamide,
N-(4-(1-(1-hydroxy-6,7-dihydro-5H-cyclopenta[c]pyridin-5-yl)-1H-pyrazolo[3,4-d]pyrimidin-6-ylamino)phenyl)methanesulfonamide,
N-(4-(1-(1-hydroxy-6,7-dihydro-5H-cyclopenta[c]pyridin-5-yl)-1H-pyrazolo[3,4-d]pyrimidin-6-ylamino)phenyl)-N-(2-hydroxyethyl)methanesulfonamide,
N-(4-(1-(1-hydroxy-6,7-dihydro-5H-cyclopenta[c]pyridin-5-yl)-1H-pyrazolo[3,4-d]pyrimidin-6-ylamino)phenyl)N-(2-morpholinoethyl)methanesulfonamide,
(1-10 above)
N-(4-(1-(4-hydroxy-6,7-dihydro-5H-cyclopenta[c]pyridin-7-yl)-1H-pyrazolo[3,4-d]pyrimidin-6-ylamino)phenyl)methanesulfonamide,
N-(4-(1-(4-hydroxy-6,7-dihydro-5H-cyclopenta[c]pyridin-7-yl)-1H-pyrazolo[3,4-d]pyrimidin-6-ylamino)phenyl)-N-(2-hydroxyethyl)methanesulfonamide,
N-(4-(1-(4-hydroxy-6,7-dihydro-5H-cyclopenta[c]pyridin-7-yl)-1H-pyrazolo[3,4-d]pyrimidin-6-ylamino)phenyl)-N-(2-morpholinoethyl)methanesulfonamide,
N-(2,3-dihydroxypropyl)-N-(4-(1-(1-hydroxy-6,7-dihydro-5H-cyclopenta[c]pyridin-5-yl)-1H-pyrazolo[3,4-d]pyrimidin-6-ylamino)phenyl)methanesulfonamide,
N-(2,3-dihydroxypropyl)-N-(4-(1-(4-hydroxy-6,7-dihydro-5H-cyclopenta[c]pyridin-7-yl)-1H-pyrazolo[3,4-d]pyrimidin-6-ylamino)phenyl)methanesulfonamide,
N-(4-(1-(4-hydroxy-6,7-dihydro-5H-cyclopenta[b]pyridin-7-yl)-1H-pyrazolo[3,4-d]pyrimidin-6-ylamino)phenyl)methanesulfonamide,
N-(4-(1-(4-hydroxy-6,7-dihydro-5H-cyclopenta[b]pyridin-7-yl)-1H-pyrazolo[3,4-d]pyrimidin-6-ylamino)phen yl)-N-(2-morpholinoethyl)methanesulfonamide,
N-(4-(1-(4-hydroxy-6,7-dihydro-5H-cyclopenta[b]pyridin-7-yl)-1H-pyrazolo[3,4-d]pyrimidin-6-ylamino)phenyl)-N-(2-hydroxyethyl)methanesulfonamide, or
N-(2,3-dihydroxypropyl)-N-(4-(1-(4-hydroxy-6,7-dihydro-5H-cyclopenta[b]pyridin-7-yl)-H-pyrazolo[3,4-d]pyrimidin-6-ylamino)phenyl)methanesulfonamide.

88. A pharmaceutical composition comprising a compound of any of claim 56, and a carrier.

89. A method of treating a subject with a protein kinase-mediated disease, such as where the disease involves Aurora kinase, a cyclin-dependant kinase, or a polo-like kinase, or a tumor or a cancer in a subject; comprising administering to said subject a pharmaceutically effective amount of a compound of claim 56.

90. A method of claim 89 comprising contacting the cell.

91. The method of claim 89, wherein the tumor or cancer is a bone cancer, brain and CNS tumor, breast cancer, breast cancer, colorectal cancer, endocrine cancer, gastrointestinal cancer, genitourinary cancer, gynecological cancer, head and neck cancer, leukemia, lung cancer, lymphoma, eye cancer, skin cancer, soft tissue sarcoma, urinary system cancer, and other types or related disorders.
